(19) United States
(12) Patent Application Publication
(10) Pub. No.: US 2022/0042075 A1
(43) Pub. Date: Feb. 10, 2022

(54) METHODS, COMPOSITIONS, AND DEVICES FOR SOLID-STATE SYNTEHSIS OF EXPANDABLE POLYMERS FO RUSE IN SINGLE MOLECULE SEQUENCINGS

(71) Applicant: Stratos Genomics, Inc., Seattle, WA (US)

(72) Inventors: Lacey Merrill, Seattle, WA (US); Marc Prindle, Seattle, WA (US); Samantha Vellucci, Seattle, WA (US); Jagadeeswaran Chandrasekar, Seattle, WA (US); Mark Stamatios Kokoris, Bothell, WA (US); Gerson Aguirre, Seattle, WA (US); John Tabone, Kirkland, WA (US); Robert N. McRuer, Mercer Island, WA (US); Michael Lee, Seattle, WA (US); Matthew Corning, Seattle, WA (US); Greg Thiessen, Seattle, WA (US); Salka Keller Barrett, Shoreline, WA (US); Christian Berrios, Seattle, WA (US); Aaron Jacobs, Seattle, WA (US); Taylor Lehmann, Seattle, WA (US)

(21) Appl. No.: 17/445,284

(22) Filed: Aug. 17, 2021

Related U.S. Application Data
(63) Continuation of application No. PCT/US2020/019131, filed on Feb. 20, 2020.
(60) Provisional application No. 62/808,768, filed on Feb. 21, 2019.
(60) Provisional application No. 62/826,805, filed on Mar. 29, 2019.

(51) Int. Cl. C12Q 1/6806; C12Q 1/6874

(52) U.S. Cl. CPC C12Q 1/6806; C12Q 1/6874

(57) ABSTRACT

Methods, compositions and devices for single molecule sequencing are provided, particularly for solid-state synthesis and processing of expandable polymers (e.g., Xpandomers), as well as methods and compositions for producing new expandable polymer constructs that provide more accurate sequence information when passed through a nanopore sensor.

### CROSS REFERENCE TO RELATED APPLICATIONS

This patent application is a continuation of International Patent Application No. PCT/US2020/019131, filed Feb. 20, 2020, which claims priority to and the benefit of United States Provisional Application No. U.S. 62/808,768, filed Feb. 21, 2019 and U.S. Provisional Application No. 62/826,805, filed Mar. 29, 2019. Each of the above patent applications is incorporated herein by reference as if set forth in its entirety.

### SEQUENCE LISTING INCORPORATION BY REFERENCE

This application contains a Sequence Listing which has been filed electronically in ASCII format and is hereby incorporated by reference in its entirety. Said ASCII copy has a file name of 870225_424WO_Sequence_Listing_ST25.txt., was created on Feb. 20, 2020, and is 5 KB in size.

### FIELD OF THE INVENTION

The present invention relates generally to new methods, compositions and devices for single molecule sequencing, and more specifically, to improved methods and devices for solid-state synthesis and processing of expandable polymers (e.g., Xpandomers), and further to methods and compositions for producing new expandable polymer constructs that provide more accurate sequence information when passed through a nanopore sensor.

### BACKGROUND

Measurement of biomolecules is a foundation of modern medicine and is broadly used in medical research, and more specifically in diagnostics and therapy, as well in drug development. Nucleic acids encode the necessary information for living things to function and reproduce, and are essentially a blueprint for life. Determining such blueprints is useful in pure research as well as in applied sciences. In medicine, sequencing can be used for diagnosis and to develop treatments for a variety of pathologies, including cancer, heart disease, autoimmune disorders, multiple sclerosis, and obesity. In industry, sequencing can be used to design improved enzymatic processes or synthetic organisms. In biology, this tool can be used to study the health of ecosystems, for example, and thus have a broad range of utility. Similarly, measurement of proteins and other biomolecules has provided markers and understanding of disease and pathogenic propagation.

An individual's unique DNA sequence provides valuable information concerning their susceptibility to certain diseases. It also provides patients with the opportunity to screen for early detection and/or to receive preventative treatment. Furthermore, given a patient's individual blueprint, clinicians will be able to administer personalized therapy to maximize drug efficacy and/or to minimize the risk of an adverse drug response. Similarly, determining the blueprint of pathogenic organisms can lead to new treatments for infectious diseases and more robust pathogen surveillance. Low cost, whole genome DNA sequencing will provide the foundation for modern medicine. To achieve this goal, sequencing technologies must continue to advance with respect to throughput, accuracy, and read length.

Over the last decade, a multitude of next generation DNA sequencing technologies have become commercially available and have dramatically reduced the cost of sequencing whole genomes. These include sequencing by synthesis (“SBS”) platforms (Illumina, Inc., 454 Life Sciences, Ion Torrent, Pacific Biosciences) and analogous ligation based platforms (Complete Genomics, Life Technologies Corporation). A number of other technologies are being developed that utilize a wide variety of sample processing and detection methods. For example, GnuBio, Inc. (Cambridge, Mass.) uses picoliter reaction vessels to control millions of discreet probe sequencing reactions, whereas Halcyon Molecular (Redwood City, Calif.) was attempting to develop technology for direct DNA measurement using a transmission electron microscope.

Nanopore based nucleic acid sequencing is a compelling approach that has been widely studied. Kasianowicz et al. (Proc. Natl. Acad. Sci. USA 93: 13770-13773, 1996) characterized single-stranded polynucleotides as they were electrically translocated through an alpha hemolysin nanopore embedded in a lipid bilayer. It was demonstrated that during polynucleotide translocation partial blockage of the nanopore aperture could be measured as a decrease in ionic current. Polynucleotide sequencing in nanopores, however, is burdened by having to resolve tightly spaced bases (0.34 nm) with small signal differences immersed in significant background noise. The measurement challenge of single base resolution in a nanopore is made more demanding due to the rapid translocation rates observed for polynucleotides, which are typically on the order of 1 base per microsecond. Translocation speed can be reduced by adjusting run parameters such as voltage, salt composition, pH, temperature, and viscosity, to name a few. However, such adjustments have been unable to reduce translocation speed to a level that allows for single base resolution.

Stratos Genomics has developed a method called Sequencing by Expansion (“SBX”) that uses a biochemical process to transcribe the sequence of DNA onto a measurable polymer called an “Xpandomer” (Kokoris et al., U.S. Pat. No. 7,939,259, “High Throughput Nucleic Acid Sequencing by Expansion”). The transcribed sequence is encoded along the Xpandomer backbone in high signal-to-noise reporters that are separated by ˜10 nm and are designed for high-signal-to-noise, well-differentiated responses. These differences provide significant performance enhancements in sequence read efficiency and accuracy of Xpandomers relative to native DNA. Xpandomers can enable several next generation DNA sequencing detection technologies and are well suited to nanopore sequencing.

Xpandomers are generated from non-natural nucleotide analogs, termed XNTPs, characterized by lengthy substituents that enable the Xpandomer backbone to be expanded following synthesis (see Published PCT Appl. No. WO2016/081871 to Kokoris et al., herein incorporated by reference in its entirety). Because of their atypical structures, polymerization of XNTPs into Xpandomers and processing of Xpandomers into expanded form for nanopore sequencing are inefficient processes, particularly in solution.

Thus, new methods and devices for improving the efficiency of synthesis and processing of Xpandomer copies of nucleic acid templates to produce a population enriched for full-length products for nanopore sequencing, as well as strategies to increase the accuracy of sequence information, would find value in the art. The present invention fulfills these needs and provides further related advantages.

All of the subject matter discussed in the Background section is not necessarily prior art and should not be assumed to be prior art merely as a result of its discussion in the Background section. Along these lines, any recognition of problems in the prior art discussed in the Background section or associated with such subject matter should not be treated as prior art unless expressly stated to be prior art. Instead, the discussion of any subject matter in the Background section should be treated as part of the inventor's approach to the particular problem, which in and of itself may also be inventive.

### SUMMARY

In brief, the present disclosure provides new methods, compositions, and devices for single-molecule nanopore sequencing. In certain embodiments, the present disclosure provides improved methods, compositions, and devices for solid-state synthesis and processing of Xpandomers and to methods and compositions for synthesizing Xpanodmers that provide more accurate sequence information.

In one aspect, the present disclosure provides a method of synthesizing a copy of a nucleic acid template on a solid substrate including the steps of a) immobilizing a linker on the solid support, in which the linker includes a first end proximal to the solid support and a second end distal to the solid support, in which the first end is coupled to a maleimide moiety and the second end is coupled to an alkyne moiety, and in which the maleimide moiety is crosslinked to the solid support; b) attaching an oligonucleotide primer to the linker, in which the oligonucleotide primer includes a nucleic acid sequence complementary to a portion of the 3′ end of the nucleic acid template, in which the 5′ end of the oligonucleotide primer is coupled to an azide moiety, and in which the azide moiety reacts with the alkyne moiety to form a triazole moiety; c) providing a reaction mixture including the nucleic acid template, a nucleic acid polymerase, nucleotide substrates or analogs thereof, a suitable buffer, and, optionally, one or more additives, in which the nucleic acid template specifically hybridizes to the oligonucleotide primer; and d) performing a primer extension reaction to produce the copy of the nucleic acid template.

In certain embodiments, the maleimide moiety is crosslinked to the solid substrate by a photo-initiated proton abstraction reaction. In other embodiments, the solid substrate is composed of polyolefin, which in alternative embodiments may be a cyclic olefin copolymer (COC) or a polypropylene. In some embodiments, the nucleic acid template is a DNA template and the copy of the DNA template is an expandable polymer, in which the expandable polymer includes a strand of non-natural nucleotide analogs, and in which the each of the non-natural nucleotide analogs is operably linked to the adjacent non-natural nucleotide analog by a phosphoramidate ester bond (e.g., an Xpandomer). In other embodiments, the linker further includes a spacer arm interposed between the first end and the second end, wherein the spacer arm includes one or more monomers of ethylene glycol. In some embodiments, the linker further includes a cleavable moiety. In other embodiments, the solid support is selected from the group consisting of a bead, a tube, a capillary, and a microfluidic chip.

In another aspect, the present disclosure provides a method of selectively modifying the 3′ end of a copy of a nucleic acid target sequence including the steps of: a) providing a first oligonucleotide with a sequence complementary to a first sequence of the nucleic acid target sequence and a second oligonucleotide with a sequence complementary to a second sequence of the nucleic acid target sequence, in which the first sequence of the nucleic acid target sequence is 3′ to the second sequence of the nucleic acid target sequence, in which the first oligonucleotide provides an extension primer for a nucleic acid polymerase and the 5′ end of the second oligonucleotide is operably linked to a dideoxy nucleoside 5′ triphosphate, wherein the dideoxy nucleoside 5′ triphosphate provides a substrate for the nucleic acid polymerase; b) providing a reaction mixture including the first and second oligonucleotides, the nucleic acid target sequence, the nucleic acid polymerase, nucleotide substrates or analogs thereof, a suitable buffer, and, optionally one or more additives, in which the first and second oligonucleotides specifically hybridize to the nucleic acid target sequence; and c) performing a primer extension reaction to produce the copy of the target sequence, in which the 5′ end of the second oligonucleotide is operably linked to the 3′ end of the copy of the nucleic acid target sequence by the nucleic acid polymerase.

In some embodiments, the dideoxy nucleoside 5′ triphosphate is operably linked to the 5′ end of the second oligonucleotide by a flexible linker. In other embodiments, the flexible linker includes one or more hexyl (C₆) monomers. In other embodiments, the second oligonucleotide includes one or more 2′methoxyribonucleic acid analogs. In yet other embodiments, the 3′ end of the second oligonucleotide is immobilized on a first solid support and in some embodiments, the method further includes the step of washing the first solid support to purify the copy of the nucleic acid target operably linked to the second oligonucleotide. In another embodiment, first oligonucleotide is immobilized to a first solid support and in some embodiments the method further includes the steps of releasing the copy of the nucleic acid target sequence from the first solid support and contacting the copy of the nucleic acid target sequence with a third oligonucleotide, in which the third oligonucleotide has a sequence that is complementary to the sequence of the second oligonucleotide, in which the third oligonucleotide specifically hybridizes with the second oligonucleotide, and in which the 5′ end of the third oligonucleotide is immobilized on a second solid support, and in yet other embodiments, further includes the step of washing the second solid support to purify the copy of the nucleic acid target sequence operably linked at the 3′ end to the second oligonucleotide. In other embodiments, the second oligonucleotide includes one or more nucleotide analogs that increase the binding affinity of the second oligonucleotide for the nucleic acid target sequence. In yet other embodiments, the second oligonucleotide is complementary to a heterologous nucleic acid sequence operably linked to the 5′ end of the nucleic target sequence. In some embodiments, the nucleic acid target sequence is single-stranded DNA and the copy of the target sequence is an expandable polymer, in which the expandable polymer includes a strand of non-natural nucleotide analogs, and in which the each of the non-natural nucleotide analogs is operably linked to the adjacent non-natural nucleotide analog by a phosphoramidate ester bond. In some embodiments, the first and second solid supports are selected from the group consisting of a bead, a tube, a capillary, and a microfluidic chip.

In another aspect, the present disclosure provides a method for producing a library of single-stranded DNA template constructs, in which the each of the template constructs includes two copies of the same strand of a DNA target sequence, including the steps of a) providing a population of DNA Y adaptors, in which each of the Y adaptors includes a first oligonucleotide and a second oligonucleotide, in which the 3′ region of the first oligonucleotide and the 5′ region of the second oligonucleotide form a double-stranded region by sequence complementarity, in which the 5′ region of the first oligonucleotide and the 3′ region of the second oligonucleotide are single-stranded and include binding sites for oligonucleotide primers, and in which the ends of the single-stranded regions of the first and second oligonucleotides are optionally immobilized on a solid substrate; b) providing a population of double-stranded DNA molecules, in which each of the double-stranded DNA molecules includes a first strand and a second strand, in which a first end of each of the double-stranded DNA molecules is compatible with the double-stranded end of the Y adaptors; c) providing a population of cap primer adaptors, in which each of the cap primer adaptors includes a first, a second, and a third oligonucleotide, in which the second oligonucleotide is interposed between the first and the third oligonucleotide, in which the first, second, and third oligonucleotides are operably linked at the 5′ ends of the first and the third oligonucleotides and the 3′ end of the second oligonucleotides by a chemical brancher, in which a portion of the sequence of the first oligonucleotide is identical to a portion of the sequence of the third oligonucleotide, in which a portion of the sequence of the second oligonucleotide is the reverse complement of the portions of the sequences of the first and third oligonucleotides, and in which the 5′ end of the second oligonucleotide and the 3′ end of the third oligonucleotide form a double-stranded region that is compatible with a second end of each of the double-stranded DNA molecules; d) ligating the second end of each of the double-stranded DNA molecules to the 5′ end of the second oligonucleotide and the 3′ end of the third oligonucleotide of one of the cap primer adaptors; e) ligating the first end of each of the double-stranded DNA molecules to the double-stranded end of one of the DNA Y adaptors; f) extending from the 3′ end of the first oligonucleotide of each of the ligated cap primer adaptors with a DNA polymerase, in which the first strand of the ligated double-stranded DNA molecule provides a template for the DNA polymerase, and in which the DNA polymerase produces a third strand that includes the reverse complement of the sequences of the first strand of the double-stranded DNA molecule and the sequence of the first oligonucleotide of the Y adaptor; and g) digesting from the 5′ end of each of the first oligonucleotides of the ligated Y adaptors with an exonuclease, in which the digesting removes the first oligonucleotide, the first strand of the double-stranded DNA molecule, and the second oligonucleotide of the cap primer adaptor to produce a single-stranded template construct, in which each of the single-stranded template constructs includes two template molecules each including the sequence of the second strand of the double-stranded DNA molecule, and in which the two template molecules are operably linked by the first and third oligonucleotides of the cap primer adaptor.

In another aspect, the present disclosure provides a library of single-stranded DNA template constructs, in which each of the template constructs includes a first and a second copy of the same strand of a DNA target sequence, in which the first and the second copies of the target sequence are operably linked; and in which the library of single-stranded DNA template constructs is produced by the above method.

In another aspect, the present disclosure provides a method of producing a library of mirrored Xpandomer molecules, in which each of the Xpandomer molecules includes two copies of the same strand of a DNA target sequence, including the steps of: a) providing the library of single-stranded DNA template constructs of the described in the paragraph above; b) providing a population of first extension oligonucleotides complementary to the single-stranded portion of the first strand of the Y adaptor and a population of second extension oligonucleotides complementary to the single-stranded portion of the second strand of the Y adaptor, and in which the first or second extension oligonucleotides are optionally immobilized on a solid substrate; c) specifically hybridizing the library of single-stranded DNA template constructs to the population of first and second extension oligonucleotides; d) providing a population of cap brancher constructs, in which the cap brancher constructs include a first oligonucleotide operably linked to a second oligonucleotide, in which the first and second oligonucleotides include sequences complementary to a portion of the sequences of the first and third oligonucleotides of the cap primer adaptor constructs, and in which the first and second oligonucleotides of the cap brancher constructs provide free 5′ nucleoside triphosphate moieties; e) specifically hybridizing the population of cap brancher constructs to the population of single-stranded DNA template constructs; and f) performing primer extension reactions to produce Xpandomer copies of the first and second copies of the DNA target sequences, in which the Xpandomer copies are operably linked by the cap brancher constructs.

In another aspect, the present disclosure provides a method for producing a library of tagged double-stranded DNA amplicons on a solid support, including the steps of: a) providing a population of double-stranded DNA molecules, in which each of the double-stranded DNA molecules includes a first strand specifically hybridized to a second strand; b) providing forward PCR primers and reverse PCR primers, in which the forward PCR primers include a first 5′ heterologous tag sequence operably linked to a 3′ sequence complementary to a portion of the 3′ end of the second stand of the double-stranded DNA molecules, and in which the reverse PCR primers include a second 5′ heterologous tag sequence operably linked to a 3′ sequence complementary to a portion of the 3′ end of the first strand of the double-stranded DNA molecules; c) performing a first PCR reaction, in which the population of double-stranded DNA molecules is amplified to produce a population of first DNA amplicon products, in which the first DNA amplicon products includes the first heterologous sequence tag on a first end and the second heterologous sequence tag on a second end; d) providing a capture oligonucleotide structure immobilized on a solid support, in which the capture oligonucleotide structure includes a first end and a second end, in which the first end is covalently attached to the solid support, in which the second end includes a capture oligonucleotide including a sequence complementary to a portion of the second heterologous sequence tag of the first population of DNA amplicon products, and in which the capture oligonucleotide structure further includes a cleavable element interposed between the first end and the capture oligonucleotide; and e) performing a second PCR reaction including the population of first DNA amplicon products, forward primers including a sequence complementary to the sequence of one of the strands of the first heterologous sequence tag, and reverse primers including a sequence complementary to one of the strands of the second heterologous sequence tag, in which a first strand of the population of first DNA amplicon products specifically hybridizes to the capture oligonucleotide, and in which the second PCR reaction produces a population of immobilized DNA amplicon products, in which a second strand of the immobilized DNA amplicon products is operably linked to the solid support.

In another aspect, the present disclosure provides a method for producing a library of single-stranded DNA template constructs, in which the each of the template constructs includes two copies of the same strand of a DNA target sequence, including the steps of: a) providing the library of DNA amplicon products immobilized on a solid support described in the paragraph above; b) providing a population of cap primer adaptors, in which each of the cap primer adaptors includes a first, a second, and a third oligonucleotide, in which the second oligonucleotide is interposed between the first and the third oligonucleotide, in which the first, second, and third oligonucleotides are operably linked at the 5′ ends of the first and the third oligonucleotides and the 3′ end of the second oligonucleotides by a chemical brancher, in which a portion of the sequence of the first oligonucleotide is identical to a portion of the sequence of the third oligonucleotide, in which a portion of the sequence of the second oligonucleotide is the reverse complement of the portions of the sequences of the first and third oligonucleotides, and in which the 5′ end of the second oligonucleotide and the 3′ end of the third oligonucleotide form a double-stranded region that is compatible with a free end of each of the tagged immobilized DNA amplicon products; c) ligating the free end of each of the immobilized DNA amplicon products to the 5′ end of the second oligonucleotide and the 3′ end of the third oligonucleotide of the cap primer adaptors; d) extending from the 3′ end of each of the first oligonucleotide of the cap primer adaptors with a DNA polymerase, in which the second strand of the immobilized DNA amplicon products provide a template for the DNA polymerase, and in which the DNA polymerase produces a third strand, wherein the third strand is a copy of the second strand; e) cleaving the cleavable element of each of the capture oligonucleotide structures, in which the cleaving releases the DNA amplicon products from the solid support and produces a free 5′ end on the second strand of each of the DNA amplicon products; and f) digesting from the free 5′ end of the cleaved second strand of each of the DNA amplicon products with an exonuclease, in which the digesting removes the second strand of the DNA amplicon product and the second oligonucleotide of the cap primer adaptor to produce a library of single-stranded template constructs, in which each of the single-stranded template constructs includes two copies of the first strand of the DNA amplicon products operably linked by the first and third oligonucleotides of the cap primer adaptor.

In another aspect, the present disclosure provides a library of single-stranded DNA template constructs, in which the each of the template constructs includes a first and a second copy of the same strand of a DNA target sequence, in which the first and second copies of the DNA target sequence are operably linked, and in which the library of single-stranded DNA template constructs is produced by the method described in the preceding paragraph.

In another aspect, the present disclosure provides a method of producing a library of mirrored Xpandomer molecules, in which each of the Xpandomer molecules includes two copies of the same strand of a DNA target sequence, including the steps of: a) providing the library of single-stranded DNA template constructs described in the preceding paragraph; b) providing a population of extension oligonucleotides complementary to the second tag of the DNA amplicon products, in which the extension oligonucleotides are immobilized on a solid substrate; c) specifically hybridizing the single-stranded DNA template constructs to the extension oligonucleotides; d) providing a population of cap brancher constructs, in which the cap brancher constructs include a first oligonucleotide operably linked to a second oligonucleotide, in which the first and second oligonucleotides include sequences complementary to a portion of the sequences of the first and third oligonucleotides of the cap primer adaptor constructs and in which the first and second oligonucleotides of the cap brancher constructs provide free 5′ nucleoside triphosphate moieties; e) specifically hybridizing the population of cap brancher constructs with the population of DNA template constructs; and f) performing primer extension reactions to produce Xpandomer copies of the first and second copies of the DNA target sequences, in which the Xpandomer copies are operably linked to the cap brancher constructs.

In some embodiments, the capture oligonucleotide structure and the extension oligonucleotides are immobilized on the same solid support, in which the extension oligonucleotides include a cleavable hairpin structure, and in which the cleavable hairpin structure is cleaved during the cleaving step to provide binding sites for the DNA amplicon products. In other embodiments, the capture oligonucleotide structure is immobilized on a first substrate of a first chamber of a microfluidic card and the extension oligonucleotides are immobilized on a second substrate of a second chamber of the microfluidic card and in which the first chamber is configured to produce the population of single-stranded DNA template constructs and the second chamber is configured to produce the population of Xpandomer copies of the single-stranded DNA template constructs. In yet other embodiments, the capture oligonucleotide structure is immobilized on a bead support and the extension oligonucleotides are immobilized on a COC chip support, in which the bead support is configured to produce the population of single-stranded DNA template constructs and the COC chip support is configured to produce the population of Xpandomer copies of the DNA template constructs. In other embodiments, the capture oligonucleotide structure and the extension oligonucleotides are immobilized on a bead support, in which the bead support is configured to produce the population of single-stranded DNA template constructs and the population of Xpandomer copies of the DNA template constructs. In another embodiment, the extension oligonucleotides are provided by a branched oligonucleotide structure, in which the branched oligonucleotide structure includes a first extension oligonucleotide operably linked to a second extension oligonucleotide by a chemical brancher, in which the first extension oligonucleotide includes a leader sequence, a concentrator sequence and a first cleavable moiety interposed between the chemical brancher and the leader and the concentrator sequences and in which the second extension oligonucleotide includes a second cleavable moiety.

The above-mentioned and additional features of the present invention and the manner of obtaining them will become apparent, and the invention will be best understood by reference to the following more detailed description. All references disclosed herein are hereby incorporated by reference in their entirety as if each was incorporated individually.

This Brief Summary has been provided to introduce certain concepts in a simplified form that are further described in detail below in the Detailed Description. Except where otherwise expressly stated, this Brief Summary is not intended to identify key or essential features of the claimed subject matter, nor is it intended to limit the scope of the claimed subject matter.

The details of one or more embodiments are set forth in the description below. The features illustrated or described in connection with one exemplary embodiment may be combined with the features of other embodiments. Thus, any of the various embodiments described herein can be combined to provide further embodiments. Aspects of the embodiments can be modified, if necessary to employ concepts of the various patents, applications and publications as identified herein to provide yet further embodiments. Other features, objects and advantages will be apparent from the description, the drawings, and the claims.

### BRIEF DESCRIPTION OF THE DRAWINGS

Exemplary features of the present disclosure, its nature and various advantages will be apparent from the accompanying drawings and the following detailed description of various embodiments. Non-limiting and non-exhaustive embodiments are described with reference to the accompanying drawings, wherein like labels or reference numbers refer to like parts throughout the various views unless otherwise specified. The sizes and relative positions of elements in the drawings are not necessarily drawn to scale. For example, the shapes of various elements are selected, enlarged, and positioned to improve drawing legibility. The particular shapes of the elements as drawn have been selected for ease of recognition in the drawings.

FIGS. 1A, 1B, 1C and 1D are condensed schematics illustrating the main features of a generalized XNTP and their use in Sequencing by Expansion (SBX).

FIG. 2 is a schematic illustrating more details of one embodiment of an XNTP.

FIG. 3 is a schematic illustrating one embodiment of an Xpandomer passing through a biological nanopore.

FIGS. 4A, 4B, 4C, 4D, and 4E are schematics illustrating exemplary embodiments of surface chemistries for solid-phase Xpandomer synthesis.

FIG. 5 is a schematic providing a generalized illustration of one embodiment of functionalization of acid-resistant beads and immobilization of an extension oligonucleotide/DNA template complex to the same.

FIG. 6A is a schematic providing a generalized illustration of the end capping methodology.

FIG. 6B is a gel showing primer extension products.

FIGS. 7A-7D are schematic illustrations of the general features of exemplary embodiments of end caps.

FIGS. 8A-8F are schematic illustrations summarizing the steps of one embodiment of solid-phase Xpandomer synthesis.

FIGS. 9A-9D are schematic illustrations summarizing the steps of another embodiment of solid-phase Xpandomer synthesis.

FIGS. 10A and 10B are schematic illustrations depicting alternative strategies to prevent polymerase “short-circuiting” during the end-capping protocol.

FIGS. 11A, 11B, and 11C are schematic illustrations summarizing the steps of one embodiment of mirrored library construction and use for Xpandomer synthesis.

FIG. 12 is a schematic illustration of the general features of one embodiment of a cap adaptor construct.

FIG. 13 summarizes one embodiment of a workflow to produce a mirrored library of Xpandomers.

FIGS. 14A and 14B are schematic illustrations summarizing the steps of one embodiment of producing an immobilized library of DNA amplicons.

FIGS. 15A and 15B are schematic illustrations summarizing the steps of one embodiment of solid-state synthesis of a library of mirrored template constructs for mirrored library Xpandomer production.

FIGS. 16A and 16B are schematic illustrations summarizing the steps of another embodiment of solid-state synthesis of a library of constructs for mirrored library Xpandomer synthesis.

FIG. 17 summarizes one embodiment of a workflow to produce a mirrored library of Xpandomers using different solid supports.

FIG. 18 is a schematic illustration of the generalized features of a branched extension oligonucleotide structure.

FIGS. 19A and 19B are schematic illustrations summarizing the steps of one embodiment of solid-state synthesis of a mirrored library of Xpandomers using a branched extension oligonucleotide.

FIG. 20 is a gel showing primer extension products.

FIG. 21A is a gel showing primer extension products.

FIG. 21B is a histogram alignment of sequencing reads from a nanopore.

FIG. 22 is a gel showing primer extension products with end capping.

FIG. 23 is a gel showing primer extension products with end capping.

FIG. 24A is a schematic illustration depicting one embodiment of a trident adaptor ligated to a library fragment.

FIG. 24B is a gel showing ligation of a trident adaptor to a library fragment.

FIG. 25A is a schematic illustration depicting one embodiment of extension and digestion reactions of an M1 mirrored library construct to produce an M3 mirrored library construct.

FIG. 25B is a gel showing products of the extension and digestion reactions.

FIG. 26A is a schematic illustration depicting one embodiment of solid-state synthesis of the M1 mirrored library construct.

FIG. 26B is a gel showing the product of solid-state synthesis of the M1 mirrored library construct.

FIG. 27 is a schematic illustration depicting one embodiment of a template for synthesis of a mirrored library Xpandomer.

FIG. 28 is a gel showing products of various stages of the mirrored library construction.

FIG. 29 is a nanopore trace showing a portion of the sequence of a mirrored library Xpandomer.

FIG. 30 is a gel showing Xpandomer products synthesized on acid-resistant magnetic beads.

FIG. 31 is a gel showing Xpandomer products synthesis and processed on acid-resistant magnetic beads.

### DETAILED DESCRIPTION OF THE INVENTION

The present invention may be understood more readily by reference to the following detailed description of preferred embodiments of the invention and the Examples included herein. Unless otherwise explained, all technical and scientific terms used herein have the same meaning as commonly understood by one of ordinary skill in the art to which this disclosure belongs.

The practice of the present invention will employ, unless otherwise indicated, conventional techniques of molecular biology, microbiology, recombinant DNA, and so forth which are within the skill of the art. Such techniques are explained fully in the literature. See e.g., Sambrook, Fritsch, and Maniatis, MOLECULAR CLONING: A LABORATORY MANUAL, Second Edition (1989), OLIGONUCLEOTIDE SYNTHESIS (M. J. Gait Ed., 1984), the series METHODS IN ENZYMOLOGY (Academic Press, Inc.), CURRENT PROTOCOLS IN MOLECULAR BIOLOGY (F. M. Ausubel, R. Brent, R. E. Kingston, D. D. Moore, J. G. Siedman, J. A. Smith, and K. Struhl, eds., 1987). All patents, patent applications, and publications mentioned herein, both supra and infra, are hereby incorporated herein by reference.

As used herein, “nucleic acids”, also called polynucleotides, are covalently linked series of nucleotides in which the 3′ position of the pentose of one nucleotide is joined by a phosphodiester group to the 5′ position of the next. A nucleic acid molecule can be deoxyribonucleic acid (DNA), ribonucleic acid (RNA), or a combination of both. DNA (deoxyribonucleic acid) and RNA (ribonucleic acid) are biologically occurring polynucleotides in which the nucleotide residues are linked in a specific sequence by phosphodiester linkages. As used herein, the terms “nucleic acid”, “polynucleotide” or “oligonucleotide” encompass any polymer compound having a linear backbone of nucleotides. Oligonucleotides, also termed oligomers, are generally shorter chained polynucleotides. Nucleic acids are generally referred to as “target nucleic acids”, “target sequence”, “template”, or “library fragment”, if targeted for sequencing.

The term “template” refers to a strand of DNA which sets the genetic sequence of new strands.

As used herein, the term “template dependent manner” is intended to refer to a process that involves the template dependent extension of a primer molecule (e.g., DNA synthesis by DNA polymerase). The term “template dependent manner” refers to polynucleotide synthesis of RNA or DNA wherein the sequence of the newly synthesized strand of polynucleotide is dictated by the well-known rules of complementary base pairing (see, for example, Watson, J. D. et al., In: Molecular Biology of the Gene, 4th Ed., W. A. Benjamin, Inc., Menlo Park, Calif. (1987)).

The term “primer”, as used herein, refers to a short strand of nucleic acid that is complementary to a sequence in another nucleic acid and serves as a starting point for DNA synthesis. Preferably the primer has at least 2, at least 3, at least 4, at least 5, at least 6, at least 7, at least 8, at least 9, at least 10, at least 11, at least 12, at least 13, at least 14, at least 15, at least 16, at least 18, at least 20, at least 25, at least 30 or more bases long.

The term “strand”, as used herein, refers to a nucleic acid made up of nucleotides covalently linked together by phosphodiester bonds. One strand of nucleic acid does not include nucleotides that are associated solely through hydrogen bonding, i.e., via base-pairing, although that strand may be base-paired with a complementary strand via hydrogen bonding. When a first stand and a second strand are base-paired through complementarity, the first strand may be referred to as the “plus” strand, the “sense” strand or the “5′ to 3′” strand and the second strand may be referred to as the “minus” strand, the “antisense” strand, or the “3′ to 5′” strand (or vice versa).

The term “3′ end”, as used herein, designates the end of a nucleotide strand that has the hydroxyl group of the third carbon in the sugar-ring of the deoxyribose at its terminus.

The term “5′ end”, as used herein, designates the end of a nucleotide strand that has the fifth carbon in the sugar-ring of the deoxyribose at its terminus.

The term “complementary” refers to the base pairing that allows the formation of a duplex between nucleotides or nucleic acids, such as for instance, between the two strands of a double-stranded DNA molecule or between an oligonucleotide primer and a primer binding site on a single-stranded nucleic acid or between an oligonucleotide probe and its complementary sequence in a DNA molecule. Complementary nucleotides are, generally, A and T (or A and U), or C and G. Two single-stranded DNA molecules are said to be substantially complementary when the nucleotides of one strand, optimally aligned and compared and with appropriate nucleotide insertions or deletions, pair with about 60% of the other strand, at least 70%, at least 80%, at least 85%, usually at least about 90% to about 95%, and even about 98% to about 100%. The degree of identity between two nucleotide regions is determined using algorithms implemented in a computer and methods which are widely known by the persons skilled in the art. The identity between two nucleotide sequences is preferably determined using the BLASTN algorithm (BLAST Manual, Altschul, S. et al., NCBI NLM NIH Bethesda, Md. 20894, Altschul, S., et al., J., 1990, Mol. Biol. 215:403-410).

“Hybridization” refers to the process in which two single-stranded polynucleotides bind non-covalently to form a stable double-stranded polynucleotide. “Hybridization conditions” will typically include salt concentrations of approximately 1 M or less, more usually less than about 500 mM and may be less than about 200 mM. A “hybridization buffer” is a buffered salt solution such as 5% SSPE, or other such buffers known in the art. Hybridization temperatures can be as low as 5° C., but are typically greater than 22° C., and more typically greater than about 30° C., and typically in excess of 37° C. Hybridizations are often performed under stringent conditions, i.e., conditions under which a primer will hybridize to its target subsequence but will not hybridize to the other, non-complementary sequences. Stringent conditions are sequence-dependent and are different in different circumstances. For example, longer fragments may require higher hybridization temperatures for specific hybridization than short fragments. As other factors may affect the stringency of hybridization, including base composition and length of the complementary strands, presence of organic solvents, and the extent of base mismatching, the combination of parameters is more important than the absolute measure of any one parameter alone. Generally stringent conditions are selected to be about 5° C., lower than the Tm for the specific sequence at a defined ionic strength and pH. Exemplary stringent conditions include a salt concentration of at least 0.01 M to no more than 1 M sodium ion concentration (or other salt) at a pH of about 7.0 to about 8.3 and a temperature of at least 25° C.

Nucleic acids are “operably linked” when they are placed into a functional relationship with each other. Generally, “operably linked” means that the nucleic acid sequences being linked are near each other. Linking maybe accomplished enzymatically, e.g., by a nucleic acid ligase or polymerase.

The expression “double stranded DNA library”, as used herein, may refer to a library that contains both strands of a molecule of DNA (i.e. the sense and antisense strands) which may be physically joined by one of their ends and forming part of the same molecule. The library of double stranded DNA molecules that may be, without limitation, genomic DNA (nuclear DNA, mitochondrial DNA, chloroplast DNA, etc.), plasmid DNA or double stranded DNA molecules obtained from single stranded nucleic acid samples (e.g. DNA, cDNA, mRNA).

As used herein, “nucleic acid polymerase” is an enzyme generally for joining 3′-OH 5′-triphosphate nucleotides, oligomers, and their analogs. Polymerases include, but are not limited to, DNA-dependent DNA polymerases, DNA-dependent RNA polymerases, RNA-dependent DNA polymerases, RNA-dependent RNA polymerases, T7 DNA polymerase, T3 DNA polymerase, T4 DNA polymerase, T7 RNA polymerase, T3 RNA polymerase, SP6 RNA polymerase, DNA polymerase 1, Klenow fragment, Thermophilus aquaticus DNA polymerase, Tth DNA polymerase, VentR® DNA polymerase (New England Biolabs), Deep VentR® DNA polymerase (New England Biolabs), Bst DNA Polymerase Large Fragment, Stoeffel Fragment, 9° N DNA Polymerase, 9° N DNA polymerase, Pfu DNA Polymerase, Tfl DNA Polymerase, Tth DNA Polymerase, RepliPHI Phi29 Polymerase, Tli DNA polymerase, eukaryotic DNA polymerase beta, telomerase, Therminator™ polymerase (New England Biolabs), KOD HiFi™ DNA polymerase (Novagen), KOD1 DNA polymerase, Q-beta replicase, terminal transferase, AMV reverse transcriptase, M-MLV reverse transcriptase, Phi6 reverse transcriptase, HIV-1 reverse transcriptase. A polymerase according to the invention can be a variant, mutant, or chimeric polymerase.

As used herein, a “DPO4-type DNA polymerase” is a DNA polymerase naturally expressed by the archaea, Sulfolobus solfataricus, or a related Y-family DNA polymerase, which generally function in the replication of damaged DNA by a process known as translesion synthesis (TLS). Y-family DNA polymerases are homologous to the DPO4 polymerase; examples include the prokaryotic enzymes, PolII, PolIV, PolV, the archaeal enzyme, Dbh, and the eukaryotic enzymes, Rev3p, Rev1p, Pol η, REV3, REV1, Pol [symbol] , and Pol κ DNA polymerases, as well as chimeras thereof.

As used herein, a “DPO4 variant” is a modified recombinant DPO4-type DNA polymerase includes one or more mutations relative to naturally-occurring wild-type DPO4-type DNA polymerases, for example, one or more mutations that increase the ability to utilize bulky nucleotide analogs as substrates or another polymerase property, and may include additional alterations or modifications over the wild-type DPO4-type DNA polymerase, such as one or more deletions, insertions, and/or fusions of additional peptide or protein sequences (e.g., for immobilizing the polymerase on a surface or otherwise tagging the polymerase enzyme). Examples of DPO4 variant polymerases according to the present invention are the variants of Sulfolobus sulfataricus DPO4 described in published PCT patent application WO2017/087281 A1 and PCT patent application nos. PCTUS2018/030972 and PCTUS2018/64794, which are hereby incorporated by reference in their entirety.

As used herein, “nucleic acid polymerase reaction” refers to an in vitro method for making a new strand of nucleic acid or elongating an existing nucleic acid (e.g., DNA or RNA) in a template dependent manner. Nucleic acid polymerase reactions, according to the invention, includes primer extension reactions, which result in the incorporation of nucleotides or nucleotide analogs to a 3′-end of the primer such that the incorporated nucleotide or nucleotide analog is complementary to the corresponding nucleotide of the target polynucleotide. The primer extension product of the nucleic acid polymerase reaction can further be used for single molecule sequencing or as templates to synthesize additional nucleic acid molecules.

The term “plurality” as used herein refers to “at least two.”

“XNTP” is an expandable, 5′ triphosphate modified nucleotide substrate compatible with template dependent enzymatic polymerization. An XNTP has two distinct functional components; namely, a nucleobase 5′-triphosphoramidate and a tether that is attached within each nucleoside triphosphoramidate at positions that allow for controlled expansion by intra-nucleotide cleavage of the phosphoramidate bond. XNTPs are exemplary “non-natural, highly substituted nucleotide analog substrates”, as used herein. Exemplary XNTPs and methods of making the same are described, e.g., in Applicants' published PCT application no. WO2016/081871, herein incorporated by reference in its entirety.

“Xpandomer intermediate” is an intermediate product (also referred to herein as a “daughter strand”) assembled from XNTPs, and is formed by polymerase-mediated template-directed assembly of XNTPs using a target nucleic acid template. The newly synthesized Xpandomer intermediate is a constrained Xpandomer. Under a process step in which the phosphoramidate bonds provided by the XNTPs are cleaved, the constrained Xpandomer is no longer constrained and is the Xpandomer product which is extended as the tethers are stretched out.

“Xpandomer” or “Xpandomer product” is a synthetic molecular construct produced by expansion of a constrained Xpandomer, which is itself synthesized by template-directed assembly of XNTP substrates. The Xpandomer is elongated relative to the target template it was produced from. It is composed of a concatenation of subunits, each subunit a motif, each motif a member of a library, comprising sequence information, a tether and optionally, a portion, or all of the substrate, all of which are derived from the formative substrate construct. The Xpandomer is designed to expand to be longer than the target template thereby lowering the linear density of the sequence information of the target template along its length. In addition, the Xpandomer optionally provides a platform for increasing the size and abundance of reporters which in turn improves signal to noise for detection. Lower linear information density and stronger signals increase the resolution and reduce sensitivity requirements to detect and decode the sequence of the template strand.

“Tether” or “tether member” refers to a polymer or molecular construct having a generally linear dimension and with an end moiety at each of two opposing ends. A tether is attached to a nucleoside triphosphoramidate with a linkage at end moiety to form an XNTP. The linkages serve to constrain the tether in a “constrained configuration”. Tethers have a “constrained configuration” and an “expanded configuration”. The constrained configuration is found in XNTPs and in the daughter strand, or Xpandomer intermediate. The constrained configuration of the tether is the precursor to the expanded configuration, as found in Xpandomer products. The transition from the constrained configuration to the expanded configuration results cleaving of selectively cleavable phosphoramidate bonds. Tethers comprise one or more reporters or reporter constructs along its length that can encode sequence information of substrates. The tether provides a means to expand the length of the Xpandomer and thereby lower the sequence information linear density.

“Tether element” or “tether segment” is a polymer having a generally linear dimension with two terminal ends, where the ends form end-linkages for concatenating the tether elements. Tether elements are segments of tether. Such polymers can include, but are not limited to: polyethylene glycols, polyglycols, polypyridines, polyisocyanides, polyisocyanates, poly(triarylmethyl)methacrylates, polyaldehydes, polypyrrolinones, polyureas, polyglycol phosphodiesters, polyacrylates, polymethacrylates, polyacrylamides, polyvinyl esters, polystyrenes, polyamides, polyurethanes, polycarbonates, polybutyrates, polybutadienes, polybutyrolactones, polypyrrolidinones, polyvinylphosphonates, polyacetamides, polysaccharides, polyhyaluranates, polyamides, polyimides, polyesters, polyethylenes, polypropylenes, polystyrenes, polycarbonates, polyterephthalates, polysilanes, polyurethanes, polyethers, polyamino acids, polyglycines, polyprolines, N-substituted polylysine, polypeptides, side-chain N-substituted peptides, poly-N-substituted glycine, peptoids, side-chain carboxyl-substituted peptides, homopeptides, oligonucleotides, ribonucleic acid oligonucleotides, deoxynucleic acid oligonucleotides, oligonucleotides modified to prevent Watson-Crick base pairing, oligonucleotide analogs, polycytidylic acid, polyadenylic acid, polyuridylic acid, polythymidine, polyphosphate, polynucleotides, polyribonucleotides, polyethylene glycol-phosphodiesters, peptide polynucleotide analogues, threosyl-polynucleotide analogues, glycol-polynucleotide analogues, morpholino-polynucleotide analogues, locked nucleotide oligomer analogues, polypeptide analogues, branched polymers, comb polymers, star polymers, dendritic polymers, random, gradient and block copolymers, anionic polymers, cationic polymers, polymers forming stem-loops, rigid segments and flexible segments.

A “reporter” is composed of one or more reporter elements. Reporters serve to parse the genetic information of the target nucleic acid.

“Reporter construct” comprises one or more reporters that can produce a detectable signal(s), wherein the detectable signal(s) generally contain sequence information. This signal information is termed the “reporter code” and is subsequently decoded into genetic sequence data. A reporter construct may also comprise tether segments or other architectural components including polymers, graft copolymers, block copolymers, affinity ligands, oligomers, haptens, aptamers, dendrimers, linkage groups or affinity binding group (e.g., biotin).

“Reporter Code” is the genetic information from a measured signal of a reporter construct. The reporter code is decoded to provide sequence-specific genetic information data.

The term “solid support”, “solid-state”, “support”, and “substrate” as used herein are used interchangeably and refer to a material or group of materials having a rigid or semi-rigid surface or surfaces. In many embodiments, at least one surface of the solid support will be substantially flat, e.g., a surface of a polymeric microfluidic card or chip. In some embodiments it may be desirable to physically separate regions of a card or chip for different reactions with, for example, etched channels, trenches, wells, raised regions, pins, or the like. According to other embodiments, the solid support(s) will take the form of insoluble beads, resins, gels, membranes, microspheres, or other geometric configurations composed of, e.g., controlled pore glass (CPG) and/or polystyrene.

The term “immobilized”, as used herein, refers to the association, attachment, or binding between a molecule (e.g. linker, adapter, oligonucleotide) and a support in a manner that provides a stable association under the conditions of elongation, amplification, ligation, and other processes as described herein. Such binding can be covalent or non-covalent. Non-covalent binding includes electrostatic, hydrophilic and hydrophobic interactions. Covalent binding is the formation of covalent bonds that are characterized by sharing of pairs of electrons between atoms. Such covalent binding can be directly between the molecule and the support or can be formed by a cross linker or by inclusion of a specific reactive group on either the support or the molecule or both. Covalent attachment of a molecule can be achieved using a binding partner, such as avidin or streptavidin, immobilized to the support and the non-covalent binding of the biotinylated molecule to the avidin or streptavidin. Immobilization may also involve a combination of covalent and non-covalent interactions.

As used herein, the term “click reaction” is recognized in the art, which describe a collection of supremely reliable and self-directed organic reactions, such as the most recognized copper catalyzed azide-alkyne [3+2] cycloaddition. Non-limiting examples of click chemistry reactions can be found, for example, in H. C. Kolb, M. G. Finn, K. B. Sharpless, Angew. Chem. Int. Ed. 2001, 40, 2004 and E. M. Sletten, C. R. Bertozzi, Angew. Chem. Int. Ed. 2009, 48, 6974, the disclosures of which are herein incorporated by reference in their entireties for all purposes.

An exemplary click chemistry reaction is the azide-alkyne Huisgen cycloaddition (e.g., using a Copper (Cu) catalyst at room temperature). (Rostovtsev, et al. 2002 Angew. Chemie Intl Ed. 41 (14): 2596-2599; Tornoe, et al. 2002 J. Org. Chem. 67 (9): 3057-3064.) Other examples of click chemistry include thiol-ene click reactions, Diels-Alder reaction and inverse electron demand Diels-Alder reaction, [4+1] cycloadditions between isonitriles (isocyanides) and tetrazines. (See, e.g., Hoyle, et al. 2010 Angew. Chemie Intl Ed. 49 (9): 1540-1573; Blackman, et al. 2008 J. Am. Chem. Soc. 130 (41): 13518-13519; Devaraj, et al. 2008 Bioconjugate Chem. 19 (12): 2297-2299; Stockmann, et al. 2011 Org. Biomol. Chem. 9, 7303-7305).

The term “alkyne” refers to a hydrocarbon having at least one carbon-carbon triple bond. As used herein, the term “terminal alkyne” refers to an alkyne wherein at least one hydrogen atom is bonded to a triply bonded carbon atom.

The term “azide” or “azido,” as used herein, refers to a group of the formula (—N₃).

The term “triazole” refers to any of the heterocyclic compounds with molecular formula C₂H₃N₃, having a five-membered ring of two carbon atoms and three nitrogen atoms. The product of a chemical click reaction between an alkyne moiety and an azide moiety is a triazole moiety.

Sequencing by Expansion

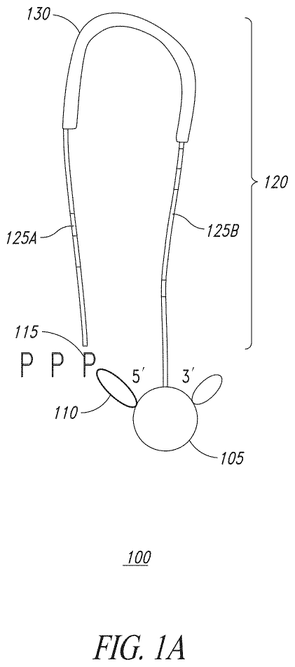

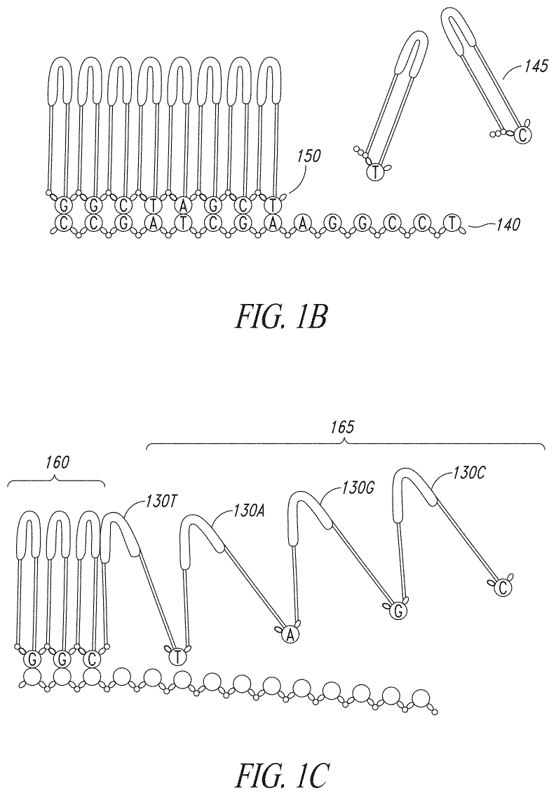

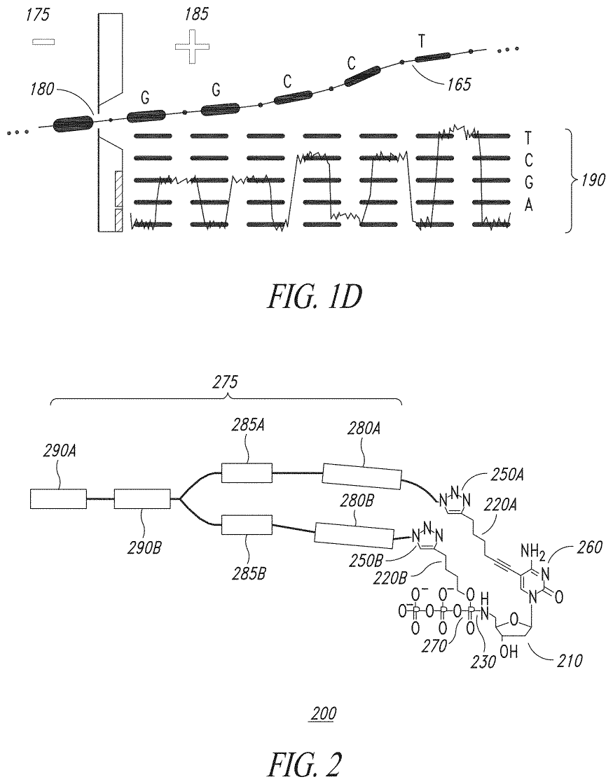

One exemplary primer extension reaction that can be enhanced by solid-state synthesis is the polymerization of the non-natural nucleotide analogs known as “XNTPs”, which forms the basis of the “Sequencing by Expansion” (SBX) protocol, developed by Stratos Genomics (see, e.g., Kokoris et al., U.S. Pat. No. 7,939,259, “High Throughput Nucleic Acid Sequencing by Expansion”). In general terms, SBX uses this biochemical polymerization to transcribe the sequence of a DNA template onto a measurable polymer called an “Xpandomer”. The transcribed sequence is encoded along the Xpandomer backbone in high signal-to-noise reporters that are separated by ˜10 nm and are designed for high-signal-to-noise, well-differentiated responses. These differences provide significant performance enhancements in sequence read efficiency and accuracy of Xpandomers relative to native DNA. A generalized overview of the SBX process is depicted in FIGS. 1A, 1B, 1C and 1D.

XNTPs are expandable, 5′ triphosphate modified nucleotide substrates compatible with template dependent enzymatic polymerization. A highly simplified XNTP is illustrated in FIG. 1A, which emphasizes the unique features of these nucleotide analogs: XNTP 100 has two distinct functional regions; namely, a selectively cleavable phosphoramidate bond 110, linking the 5′ α-phosphate 115 to the nucleobase 105, and a tether 120 that is attached within the nucleoside triphosphoramidate at positions that allow for controlled expansion by intra-nucleotide cleavage of the phosphoramidate bond. The tether of the XNTP is comprised of linker arm moieties 125A and 125B separated by the selectively cleavable phosphoramidate bond. Each linker attaches to one end of a reporter 130 via a linking group (LG), as disclosed in U.S. Pat. No. 8,324,360 to Kokoris et al., which is herein incorporated by reference in its entirety. XNTP 100 is illustrated in the “constrained configuration”, characteristic of the XNTP substrates and the daughter strand following polymerization. The constrained configuration of polymerized XNTPs is the precursor to the expanded configuration, as found in Xpandomer products. The transition from the constrained configuration to the expanded configuration occurs upon scission of the P—N bond of the phosphoramidate within the primary backbone of the daughter strand.

Synthesis of an Xpandomer is summarized in FIGS. 1B and 1C. During assembly, the monomeric XNTP substrates 145 (XATP, XCTP, XGTP and XTTP) are polymerized on the extendable terminus of a nascent daughter strand 150 by a process of template-directed polymerization using single-stranded template (SEQ ID NO:1) 140 as a guide. Generally, this process is initiated from a primer and proceeds in the 5′ to 3′ direction. Generally, a DNA polymerase or other polymerase is used to form the daughter strand, and conditions are selected so that a complimentary copy of the template strand is obtained. After the daughter strand is synthesized, the coupled tethers comprise the constrained Xpandomer that further comprises the daughter strand. Tethers in the daughter strand have the “constrained configuration” of the XNTP substrates. The constrained configuration of the tether is the precursor to the expanded configuration, as found the Xpandomer product.

As shown in FIG. 1C, the transition from the constrained configuration 160 to the expanded configuration 165 results from cleavage of the selectively cleavable phosphoramidate bonds (illustrated for simplicity by the unshaded ovals) within the primary backbone of the daughter strand. In this embodiment, the tethers comprise one or more reporters or reporter constructs, 130A, 130C, 130G, or 130T, specific for the nucleobase to which they are linked, thereby encoding the sequence information of the template. In this manner, the tethers provide a means to expand the length of the Xpandomer and lower the linear density of the sequence information of the parent strand.

FIG. 1D illustrates an Xpandomer 165 translocating through a nanopore 180, from the cis reservoir 175 to the trans reservoir 185. Upon passage through the nanopore, each of the reporters of the linearized Xpandomer (in this illustration, labeled “G”, “C” and “T”) generates a distinct and reproducible electronic signal (illustrated by superimposed trace 190), specific for the nucleobase to which it is linked.

FIG. 2 depicts the generalized structure of an XNTP in more detail. XNTP 200 is comprised of nucleobase triphosphoramidate 210 with linker arm moieties 220A and 220B separated by selectively cleavable phosphoramidate bond 230. Tethers are joined to the nucleoside triphosphoramidate at linking groups 250A and 250B, wherein a first tether end is joined to the heterocycle 260 (represented here by cytosine, though the heterocycle may be any one of the four standard nucleobases, A, C, G, or T) and the second tether end is joined to the alpha phosphate 270 of the nucleobase backbone. The skilled artisan will appreciate that many suitable coupling chemistries known in the art may be used to form the final XNTP substrate product, for example, tether conjugation may be accomplished through a triazole linkage.

In this embodiment, tether 275 is comprised of several functional elements, including enhancers 280A and 280B, reporter codes 285A and 285B, and translation control elements (TCEs) 290A and 290B. Each of these features performs a unique function during translocation of the Xpandomer through a nanopore and generation of a unique and reproducible electronic signal. Tether 275 is designed for translocation control by hybridization (TCH). As depicted, the TCEs provide a region of hybridization which can be duplexed to a complementary oligomer (CO) and are positioned adjacent to the reporter codes. Different reporter codes are sized to block ion flow through a nanopore at different measureable levels. Specific reporter codes can be efficiently synthesized using phosphoramidite chemistry typically used for oligonucleotide synthesis. Reporters can be designed by selecting a sequence of specific phosphoramidites from commercially available libraries. Such libraries include but are not limited to polyethylene glycol with lengths of 1 to 12 or more ethylene glycol units, aliphatic with lengths of 1 to 12 or more carbon units, deoxyadenosine (A), deoxycytosine (C), deoxyguanodine (G), deoxythymine (T), abasic (Q). The duplexed TCEs associated with the reporter codes also contribute to the ion current blockage, thus the combination of the reporter code and the TCE can be referred to as a “reporter”. Following the reporter codes are the enhancers, which in one embodiment comprise spermine polymers.

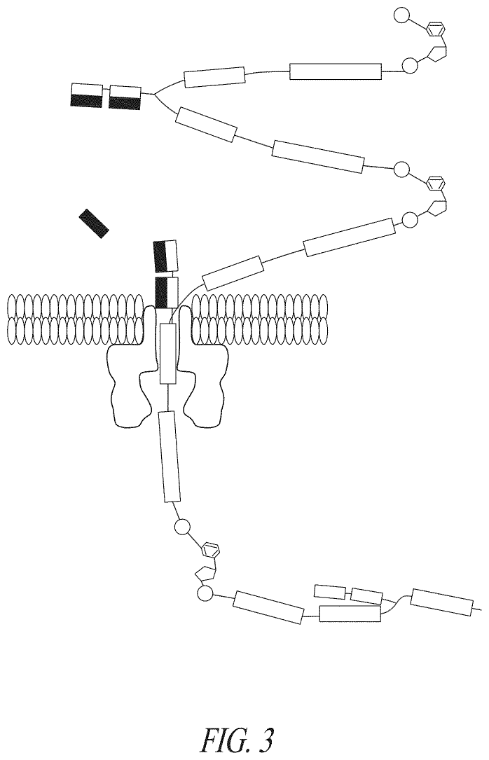

FIG. 3 shows one embodiment of a cleaved Xpandomer in the process of translocating an α-hemolysin nanopore. This biological nanopore is embedded into a lipid bilayer membrane which separates and electrically isolates two reservoirs of electrolytes. A typical electrolyte has 1 molar KCl buffered to a pH of 7.0. When a small voltage, typically 100 mV, is applied across the bilayer, the nanopore constricts the flow of ion current and is the primary resistance in the circuit. Xpandomer reporters are designed to give specific ion current blockage levels and sequence information can be read by measuring the sequence of ion current levels as the sequence of reporters translocate the nanopore.

The α-hemolysin nanopore is typically oriented so translocation occurs by entering the vestibule side and exiting the stem side. As shown in FIG. 3, the nanopore is oriented to capture the Xpandomer from the stem side first. This orientation is advantageous using the TCH method because it causes fewer blockage artifacts that occur when entering vestibule first. Unless indicated otherwise, stem side first will be the assumed translocation direction. As the Xpandomer translocates, a reporter enters the stem until its duplexed TCE stops at the stem entrance. The duplex is ˜2.4 nm in diameter whereas the stem entrance is ˜2.2 nm so the reporter is held in the stem until the complimentary strand 395 of the duplex disassociates (releases) whereupon translocation proceeds to the next reporter. The free complementary strand is highly disfavored from entering the nanopore because the Xpandomer is still translocating and diffuses away from the pore.

In one embodiment, each member of a reporter code (following the duplex) is formed by an ordered choice of phosphoramidites that can be selected from many commercial libraries. Each constituent phosphoramidite contributes to the net ion resistance according to its position in the nanopore (located after the duplex stop), its displacement, its charge, its interaction with the nanopore, its chemical and thermal environment and other factors. The charge on each phosphoramidite is due, in part, to the phosphate ion which has a nominal charge of −1 but is effectively reduced by counterion shielding. The force pulling on the duplex is due to these effective charges along the reporter which are acted upon by the local electric fields. Since each reporter can have a different charge distribution, it can exert a different force on the duplex for a given applied voltage. The force transmitted along the reporter backbone also serves to stretch the reporter out to give a repeatable blocking response.

For sequencing, protein nanopores are prepared by inserting α-hemolysin into a DPhPE/hexadecane bilayer member in buffer B1, containing 2 M NH₄Cl and 100 mM HEPES, pH 7.4. The cis well is perfused with buffer B2, containing 0.4 M NH₄Cl, 0.6 M GuCl, and 100 mM HEPES, pH 7.4. The Xpandomer sample is heated to 70° C. for 2 minutes, cooled completely, then a 2 μL sample is added to the cis well. A voltage pulse of 90 mV/390 mV/10 μs is then applied and data is acquired via Labview acquisition software.

Sequence data is analyzed by histogram display of the population of sequence reads from a single SBX reaction. The analysis software aligns each sequence read to the sequence of the template and trims the extent of the sequence at the end of the reads that does not align with the correct template sequence.

### 2. Specific Embodiments of the Invention

The present invention may employ particular methods, devices, and compositions as described in the following exemplary embodiments.

A. Solid-State Synthesis

The Sequencing by Expansion (SBX) methodology developed by the inventors provides significant performance enhancements in sequence read efficiency and accuracy of Xpandomers relative to native DNA. However, samples enriched for high-quality, full-length Xpandomer copies of template DNA can be difficult to produce in solution. Advantageously, through trial and error, the inventors have found that the efficiency of synthesis and/or processing of full-length Xpandomers can be increased by adapting various steps of the workflow (e.g., the primer extension reaction and/or post-synthetic processing steps) to a solid support. Solid-state platforms have been found to improve optimization of various reaction conditions.

Solid-state synthesis of Xpandomers may be carried out using any suitable support platform known in the art. In certain embodiments, the solid-state support may be a conventional bead, tube, capillary, or microfluidic chip or card. As discussed further herein, in some embodiments of the invention, an oligonucleotide primer, i.e. an extension, or “E-oligo”, is bound to the support to initiate solid-state Xpandomer synthesis.

Surface Chemistries

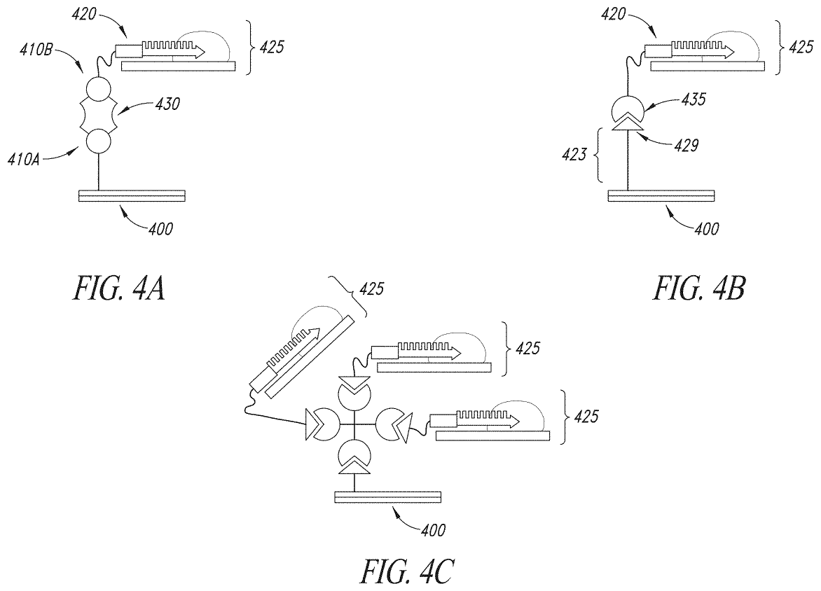

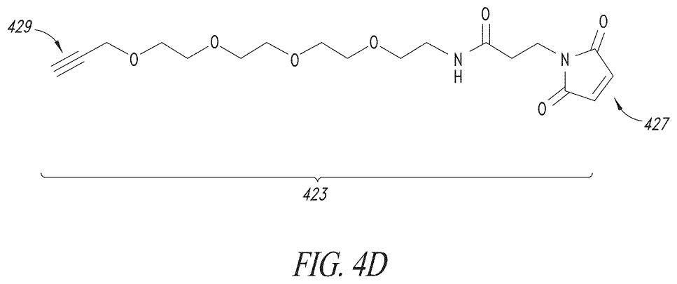

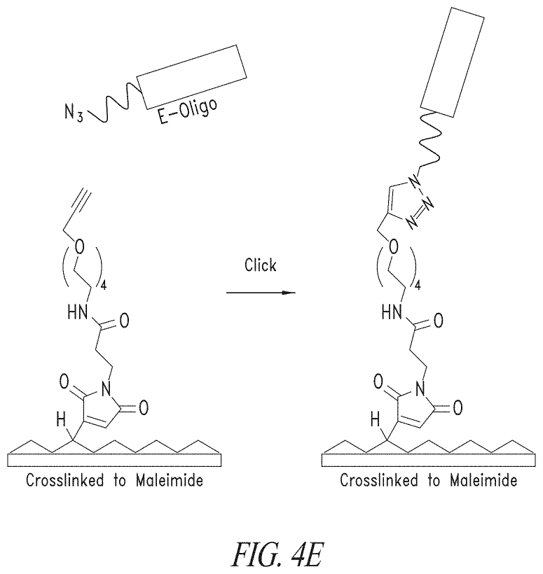

Multiple surface chemistries may be used to immobilize an oligonucleotide or an oligonucleotide/template complex on a solid support. Certain exemplary embodiments of suitable surface chemistries are illustrated in FIGS. 4A-4E. The embodiment depicted in FIG. 4A employs conventional streptavidin/biotin interaction chemistry and shows functionalization of a solid support 400 with a linker that includes terminal biotin moiety 410A. In this embodiment, the 5′ end of an oligonucleotide primer 420 is bound to a second linker that includes terminal biotin moiety 410B. Attachment of a primer-template complex 425 (in this depiction illustrating polymerase-mediated Xpandomer synthesis) to the support is mediated by streptavidin moiety 430. The linker moieties disclosed herein may be of sufficient length to connect the oligonucleotide to the support such that the support does not significantly interfere with the overall binding and recognition of the oligonucleotide by a complementary oligonucleotide or a nucleic acid replication enzyme. Thus, the linker can also comprise a spacer unit. The spacer distances, for example, the oligonucleotide from a cleavage site or label.

Alternatively, the embodiment depicted in FIG. 4B illustrates immobilization of a primer-template complex 425 to a solid support (i.e., “substrate”) 400 by covalent linkage of the primer to the substrate via a click reaction. In this embodiment, the covalent linkage is mediated by a maleimide-PEG-alkyne linker 423 that is crosslinked to the solid support. An alkyne moiety 429 provided by the end of the linker distal to the substrate is capable of reacting with an azide group 435 provided by the 5′ end of the primer. The ability to utilize simple click chemistry to immobilize nucleic acids on a substrate offers advantages over conventional solid-state nucleic acid synthesis protocols. For example, nucleic acids may be presynthesized (e.g., either chemically or enzymatically) and purified prior to click conjugation. In addition, combinations of different oligonucleotides can be immobilized on a single support. Multiple configurations of oligonucleotide structures bound to a solid-support are contemplated by the present invention. FIG. 4C illustrates how a dendrimer of primer-template complexes can be formed on a support by click chemistry, as discussed herein.

Any suitable linker that provides a maleimide moiety on a first end and an alkyne moiety on a second end may be used according to the present invention. The chemical chain between the two reactive groups of the linker may be referred to herein as the “spacer arm”. The length of the spacer arm determines how flexible the conjugate will be and can be optimized for particular applications. Typically, the spacer arms include hydrocarbon chains or polyethylene glycol (PEG) chains. FIG. 4D illustrates an exemplary maleimide-PEG-alkyne linker 423, propargyl-PEG4-maleimide, that provides alkyne moiety 429 and maleimide moiety 427. FIG. 4E illustrates how an extension oligonucleotide with a terminal azide moiety linked to the 5′ end can be immobilized on a solid support by a click reaction that produces a covalent linkage. In this embodiment, the solid support has been functionalized by crosslinking a linker that includes a terminal maleimide moiety at the end proximal to the support and a terminal alkyne group at the end distal to the support.

According to the present invention, a maleimide moiety can be converted into a reactive group and subsequently crosslinked to a solid surface, e.g., a polyolefin surface, via a catalyst-free photochemical (e.g., photo-initiated) proton abstraction reaction. This reaction simplifies the initiation step that conventional conjugation methodologies rely on. Conventional crosslinking technologies teach that the maleimide chemical group is sulfhydral-reactive, targeting (—SH) functional groups. However, the inventors have advantageously discovered that the maleimide group can be crosslinked to a rigid polyolefin substrate following activation via a proton abstraction reaction. Importantly, the maleimide-mediated crosslink has been found to be stable under acidic and conditions as well as during a click reaction. Suitable polyolefin surfaces include, but are not limited to, substrates manufactured from polypropylene or cyclic olefin copolymer (COC).

To functionalize a substrate, e.g., a COC chip, with an alkyne moiety, an exemplary catalyst-free photochemical proton abstraction reaction may include the following steps: 1) priming the chip with an organic solvent, such as DMSO or DMF; 2) adding a linker with a maleimide moiety on one end, such as propargyl maleimide, solubilized in, e.g., DMSO and water; 3) incubating the chip under a UV lamp; 3) washing the chip with a series of solvents, which in certain embodiments may include DMSO, DMF, and a solution of Na₂HPO₄, Tween-20, and SDS; and 4) washing the chip with aqueous solutions such as water and/or PBS prior to the click reaction.

Although these embodiments illustrate the 5′ end of an extension oligonucleotide, i.e., primer linked to the support, it is to be understood that, in alternative embodiments, the surface chemistries can be adapted to link the 3′ of an oligonucleotide, e.g., the terminal oligonucleotide of an end cap structure discussed further herein (or the 5′ end of an oligonucleotide with a sequence that is the reverse complement of a terminal oligonucleotide) to the support.

In certain embodiments, the linkage between the oligonucleotide and the solid support is cleavable, enabling primer extension products to be released from the support following synthesis. Cleavable linkers and methods of cleaving such linkers are known and can be employed in the provided methods using the knowledge of those of skill in the art. For example, the cleavable linker can be cleaved by an enzyme, a catalyst, a chemical compound, temperature, electromagnetic radiation or light. Optionally, the cleavable linker includes a moiety hydrolysable by beta-elimination, a moiety cleavable by acid hydrolysis, an enzymatically cleavable moiety, or a photocleavable moiety. In some embodiments, a suitable cleavable moiety is a photocleavable (PC) spacer or linker phosphoramidite available from Glen Research.

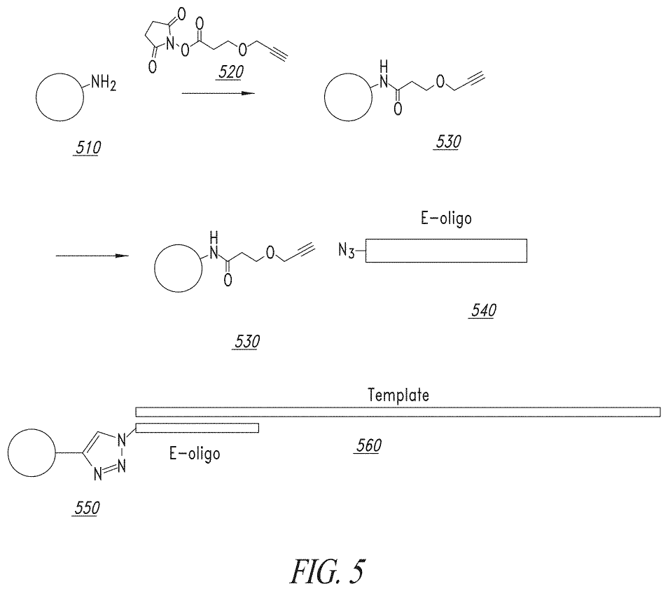

The inventors have advantageously found that solid-state synthesis and processing of Xpandomers allows for optimization of many steps in the workflow, such that nanopore sequence reads over 400 bases have been obtained. In certain embodiments, solid-state synthesis may be conducted using acid-resistant magnetic beads as a support. The geometry of the bead structure provides several advantages, including favorable template-binding and rapid in-solution reaction kinetics, increased surface area, magnetic collection, and the like. The acid-resistance of the beads makes them a particularly suitable support for Xpandomer processing reactions. One embodiment of a method of preparing acid-resistant magnetic beads for Xpandomer synthesis is illustrated in FIG. 5. Here, acid-resistant magnetic bead 510 (e.g., TurboBeads® Peg amine) are functionalized with linker 520 to produce functionalized beads 530, providing a terminal alkyne group. The beads may be functionalized using any form of amine-type coupling or chemical condensation. In one embodiment, the beads may be functionalized by NHS-ester conjugation with the amine provided by the surface of the bead. Through click chemistry, an extension oligonucleotide (“E-oligo”) 540 providing a 5′ azide moiety is covalently attached to the functionalized bead 530 to produce support-bound E-oligo 550. The bead-bound E-oligo can be hybridized to a single-stranded template 560 for, e.g., a primer extension reaction to produce an Xpandomer copy of the template. Advantageously, subsequent Xpandomer processing steps, including acid-mediated cleavage of the phosphoramidate bonds, can be carried-out on the same bead support.

End Capping

In this embodiment, a single-stranded copy of a nucleic acid template is operably linked (e.g., joined or attached) at the 3′-end to the 5′ end of an oligonucleotide “cap” that is specifically hybridized to a portion of the template. Linkage of the single-stranded copy to the oligonucleotide cap is mediated by a nucleic acid polymerase as it reaches the 5′ end of the oligonucleotide cap during template-dependent. The oligonucleotide cap is referred to herein alternatively as an “end cap”, a “capped blocker oligonucleotide”, or an “end tag”. The end cap functions as a molecular tag to identify and/or isolate copies of a nucleic acid template that have a defined length from a heterogenous population of products that may include copies of an undesirable length, e.g., incomplete or truncated products.

In alternative embodiments, the template nucleic acid may be a DNA molecule or an RNA molecule. The end cap may be designed to hybridize to any portion (i.e., to an “end cap target sequence”) of the template nucleic acid so as to selectively modify, e.g., “tag” a copy of a region of the template with a defined or desired length, i.e. a “target sequence”. In some embodiments, the end cap is designed to hybridize to a sequence near the 5′ end of the target sequence, so as to “tag” a complete, or nearly complete, copy of the target sequence. In some embodiments, the end cap target sequence is a portion of the natural nucleic acid sequence of the template nucleic acid. In other embodiments, the end cap target sequence is a heterologous sequence (e.g., an adaptor or linker) that is joined or ligated to the template nucleic acid.

In certain embodiments, the copy of the single-stranded nucleic acid template is an Xpandomer and the end cap is designed to hybridize to the 5′ end of a library fragment of template DNA. Advantageously, a population of Xpandomer products enriched for full-length copies of the library fragment provides improved sequence information, or “reads”, from the nanopore-based sequencing systems of the present invention.

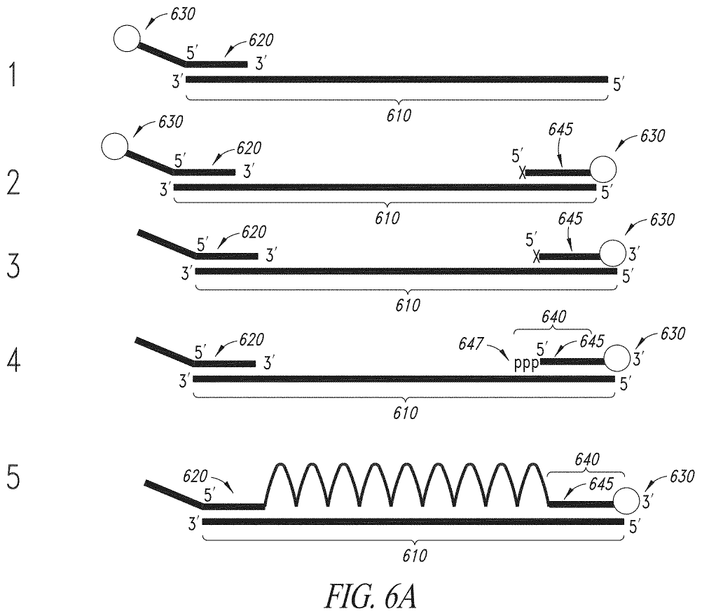

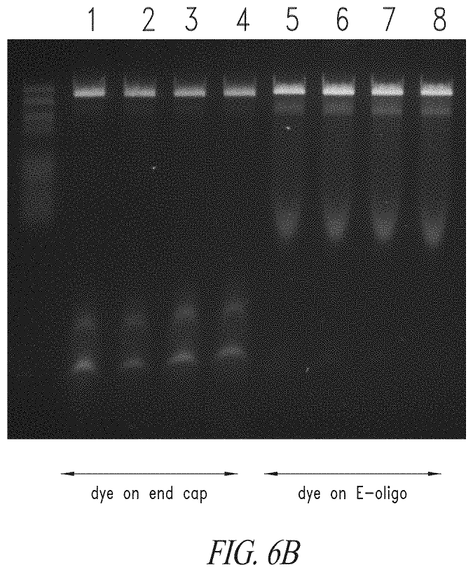

An overview of one embodiment of an end-capping strategy is illustrated in simplified form in FIG. 6A. In this embodiment, end-capping enables selective tagging of Xpandomer copies of a DNA target sequence, herein represented by target sequence template 610. Xpandomers are synthesized by a primer extension reaction initiated from an oligonucleotide primer 620 (i.e., the extension, or “E-oligo”) hybridized to the single-stranded template with a suitable DNA polymerase, XNTP substrates and other extension reagents and additives. The inventors have found that variants of DPO4 polymerase are capable of utilizing XNTPs as substrates to synthesize Xpandomers in a template-dependent manner, particularly when the primer extension reactions include one or more PEM additives (PEM additives are described, e.g., in Applicants' pending patent application no. PCT/US18/67763, entitled “Enhancement of Nucleic Acid Polymerization by Aromatic Compounds”, herein incorporated by reference in its entirety). Primer extension products may be visualized by gel electrophoresis when an oligonucleotide incorporated into the extension product is linked to a detectable dye 630.

The general features of one embodiment of an end cap structure are illustrated in schematic number 4 of FIG. 6A. In this embodiment, the end cap 640 includes a terminal oligonucleotide 645 (which may be referred to herein as the “blocker” oligonucleotide) that is complementary to, and specifically hybridizes with, a sequence near the 5′ end of the target sequence template. The end cap also includes a 5′ triphosphate group 647 bound to a dideoxyribonucleoside analog (i.e., the “cap”) that is capable of being utilized as a substrate by the DNA polymerase. During a primer extension reaction, e.g., an Xpandomer synthesis reaction, the DNA polymerase synthesizes the growing Xpandomer from the bound extension oligonucleotide in a template-dependent manner. Upon reaching the end of the template, the DNA polymerase encounters the end cap and joins the 5′ end of the terminal oligonucleotide to the 3′ end of the Xpandomer through formation of a phosphodiester bond between the triphosphate group of the cap and the 3′ terminal XNMP of the Xpandomer as depicted in the fifth cartoon. In contrast, terminal oligonucleotides lacking a free 5′ triphosphate group, as depicted oligonucleotide 645 in the third cartoon of FIG. 6A, are incapable of being joined to the Xpandomer by the DNA polymerase.

In certain embodiments, the end cap may be linked to a detectable dye 630 to visualize end-capped copies of the target sequence by, e.g., gel electrophoresis. FIG. 6B shows an exemplary gel in which Xpandomer copies of a 100mer template are labeled either on the end cap (lanes 1-4, corresponding to the fourth cartoon of FIG. 6A), or the primer (lanes 5-8, corresponding to the first cartoon of FIG. 6A). End-capping is dependent on the availability of the 5′ nucleoside triphosphate group bound to the terminal oligonucleotide, as indicated by the absence of fluorescent signal when primer extension reaction are conducted with a blocker oligonucleotide 645 lacking a free 5′ triphosphate group (data not shown, corresponding to the second and third cartoon of FIG. 6A).

In some embodiments, the end cap, or an oligonucleotide complementary to the to the terminal oligonucleotide of the end cap, may be linked to a solid support to enable isolation or purification (e.g., “capture”) of full-length Xpandomer products, as described in further detail herein.

The terminal, or “blocker”, oligonucleotide is designed to hybridize strongly with the end cap target sequence in the template nucleic acid. Features such as the length of the oligonucleotide and/or the chemical structure of one or more nucleotides monomers of the oligonucleotide may be optimized to achieve the desired hybridization strength. In general terms, the melting temperature of terminal oligonucleotide-target sequence template will be at least 37° C. for optimal hybrid formation, though lower melting temperatures are possible. In certain embodiments, the length of the terminal oligonucleotide is from around 10 to around 30 nucleotides. In some embodiments, nucleotide analogs, such one or more 2′ methoxyribonucleotides, LNAs (i.e. “locked” nucleic acid analogs), or G clamps are incorporated into the terminal oligonucleotide to increase binding efficiency. In one embodiment, substantially all of the nucleotides of the terminal nucleotide at 2′methoxyribonucleotide.

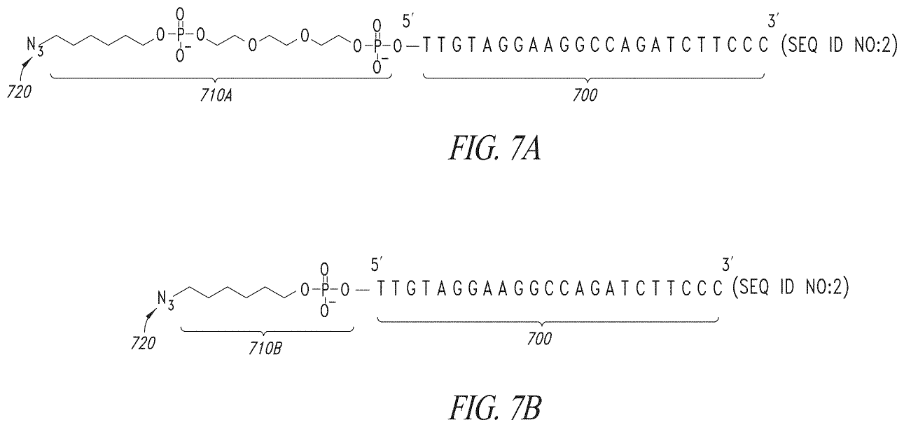

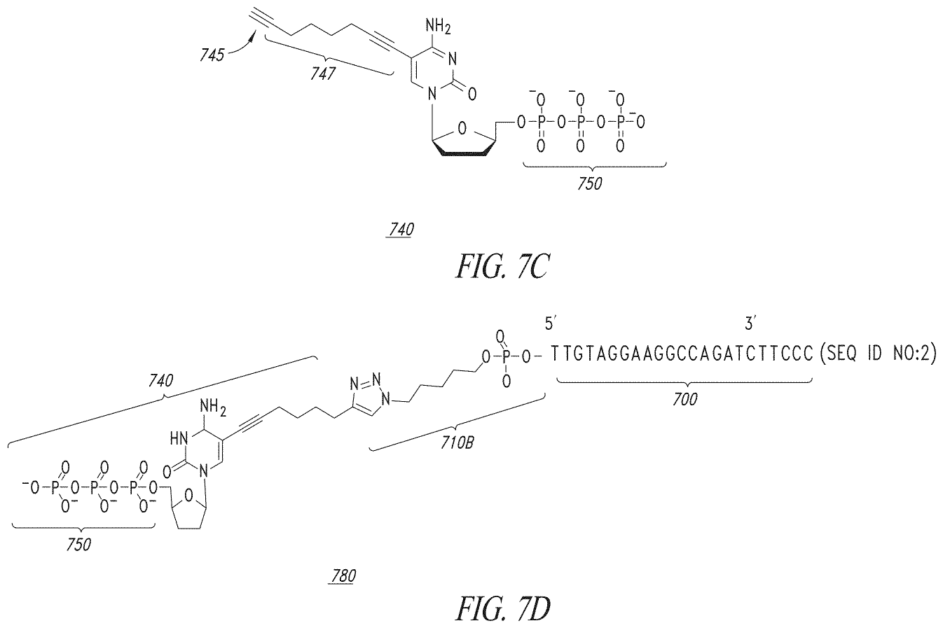

Details of certain features of exemplary end cap structures are illustrated in FIGS. 7A-7D. FIGS. 7A and 7B, depict terminal oligonucleotide (SEQ ID NO:2) 700 in which the 5′ end of the oligonucleotide is joined to a flexible linker 710. The flexible linker includes a terminal azide moiety 720 that provides a substrate for a click reaction that enables covalent linkage to a modified 5′ nucleoside triphosphate cap (i.e., the “cap), as further described with reference to FIG. 7C. Exemplary embodiments of flexible linkers 710A and 710B bound to the 5′ end of a 23mer terminal oligonucleotide 700 are illustrated in FIGS. 7A and 7B, respectively. The flexible linker may be an inert linear polymer comprised of, e.g., alkyl and/or PEG moieties of suitable lengths. In one embodiment, the flexible linker is formed from a C6 bromohex phosphoramidite. In some embodiments, the 5′ end of the oligonucleotide may include one or more G clamp nucleotide analogs

In an exemplary method of synthesis, the terminal oligonucleotide is synthesized by conventional automated phosphoramidite chemistry during which the 5′-hydroxyl of the completed oligonucleotide is coupled to a bromo-hexyl phosphoramidite (available from, e.g., Glen Research). The solid support is treated with sodium azide to convert the bromo group to an azide. Finally, the oligonucleotide is deprotected and cleaved from the solid support to provide an azido oligonucleotide, as illustrated in FIG. 7B.

FIG. 7C illustrates one embodiment of a modified 5′ nucleoside triphosphate cap 740, designated herein as “ddNTP-O” (represented by ddCTP-0 in this depiction). The heterocycle moiety of the cap is modified with a terminal alkyne moiety 745 linked via an octadiynle arm 747 to mediate attachment to the azide of the terminal oligonucleotide via a click reaction. In certain embodiments, the alkynyl nucleoside triphosphate (i.e., cap 740) of the resulting end cap is capable of base pairing with the template at the 5′ end of the terminal oligonucleotide. The alkynyl nucleoside triphosphate cap may be synthesized using the method described by Ludwig and Eckstein or other methods of 5′-triphosphate synthesis see, e.g., A. R. Kore, A. R, Srinivasan B., Recent Advances in the Syntheses of Nucleoside Triphosphates, Current Organic Synthesis, 10(6), 903-34 (2013), which is herein incorporated by reference in its entirety.

FIG. 7D illustrates one embodiment of a complete end cap structure 780 formed by a click reaction to operably link triphosphate cap 740 (i.e. alkynyl nucleoside triphosphate cap) to terminal oligonucleotide (SEQ ID NO:2) 700. Without being bound by theory, it is hypothesized that the flexible linker 710B of the end cap provides sufficient steric flexibility, or degrees of freedom, to the structure such that triphosphate group 750 can enter the active site of the DNA polymerase and function as a substrate for the formation of a phosphodiester bond between the end cap and the 3′ end of the Xpandomer during the primer extension reaction. Variants of DPO4 DNA polymerase are particularly well suited for joining the end cap structure to the 3′ end of an Xpandomer.

In certain embodiments of the present invention, alternative end cap structures and means of joining a terminal oligonucleotide to the 3′ end of an Xpandomer are contemplated. In one embodiment, a psoralen bridge ligation method is utilized. Briefly, the 5′ end of the terminal oligonucleotide is modified to present a psoralen moiety, which on exposure to ultraviolet (UVA) radiation can form monoadducts and covalent interstrand cross-links (ICL) with thymines. Thus, the psoralen-modified terminal oligonucleotide may be chemically cross-linked to a 3′ thymine in an Xpandomer upon exposure to UVA radiation. Advantageously, the psoralen bridge is resistant to acid cleavage.

In other embodiments, the psoralen-modified terminal oligonucleotide may include other features to enable attachment to and release from a solid substrate. For example, the 3′ end of the oligonucleotide may include a linker nucleic acid sequence comprising a cleavage site for a nuclease enzyme. In some embodiments, the cleavage site is recognized and cleaved by RNase. Any suitable RNase recognition site may be used, e.g., for RNase A, RNase H, or RNase Ti. In other embodiments, the cleavage site is recognized and cleaved by a nicking endonuclease or trypsin. When bound to a solid support via the 3′ end of the linker, the terminal oligonucleotide may be selectively released by enzymatic treatment with the appropriate nuclease.

End Tagging

As an alternative strategy to end-capping, the inventors have devised compositions and methods to operably link (e.g., join or covalently attach) a leader sequence to the 3′ end of an Xpandomer following synthesis. In this manner, only substantially full-length Xpandomers will include a 3′ leader sequence, which is required for threading the Xpandomer through a nanopre sensor. In one embodiment, the end tag structure is essentially a modified Xpandomer in which the reporter code elements are replaced by leader and enhancer elements and the translocation control elements are replace by polyG oligomers. Both the phosphoramidate bond and the polyG oligomer elements of the end tag are acid-labile. Thus, upon acid treatment, the 5′ half of the end tag will remain associated with the Xpandomer, including one of the leader and enhancer elements, which enables nanopore threading from the 3′ end of the Xpandomer.

In one embodiment, a method of end-tagging an Xpandomer may include the steps of: 1) performing solid-state Xpandomer synthesis in which the substrate-bound extension oligonucleotide lacks a leader and an enhancer sequence; 2) running the extension reaction for a period of time sufficient to provide a population of substantially full-length Xpandomer products; 3) washing the substrate-bound products to remove all extension reagents; and 4) adding to the substrate the end tag structure and other reaction components necessary for polymerase-mediated attachment of the end tag to the 3′ end of the Xpandomer. In some embodiments, the method may include the steps of hybridizing a terminal blocker nucleotide to the template prior to the extension reaction and removing the terminal blocker nucleotide following extension and prior to washing and performing the end tag addition reaction.

B. Solid-State Synthesis with End Capping

The end-capping methodology described herein can be integrated with solid-state Xpandomer synthesis workflows using any suitable support platform known in the art. In certain embodiments, the solid-state support may be a conventional bead, tube, capillary, or microfluidic chip. In one embodiment, the solid support is an acid-resistant magnetic bead. As discussed further herein, in some embodiments of the invention, an oligonucleotide primer may be bound to the support. In other embodiments, the terminal oligonucleotide of the end cap, or its reverse complement, may be bound to the support.

Away from Support (AFS) Xpandomer Synthesis Workflow

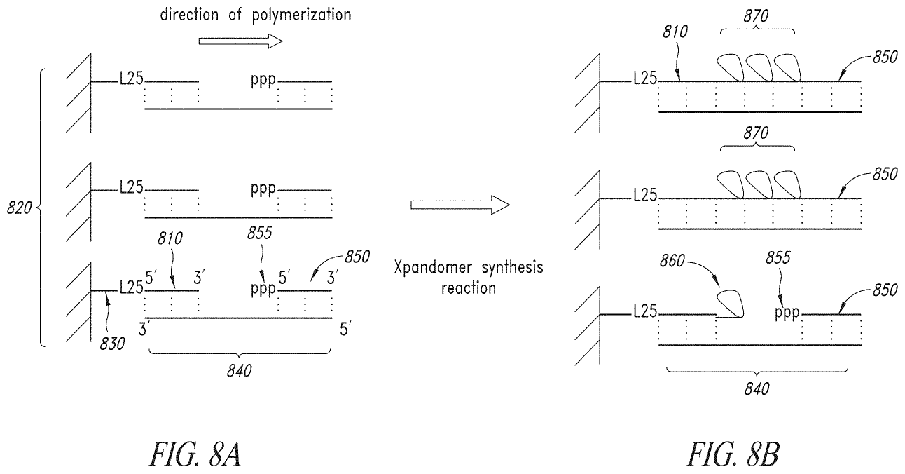

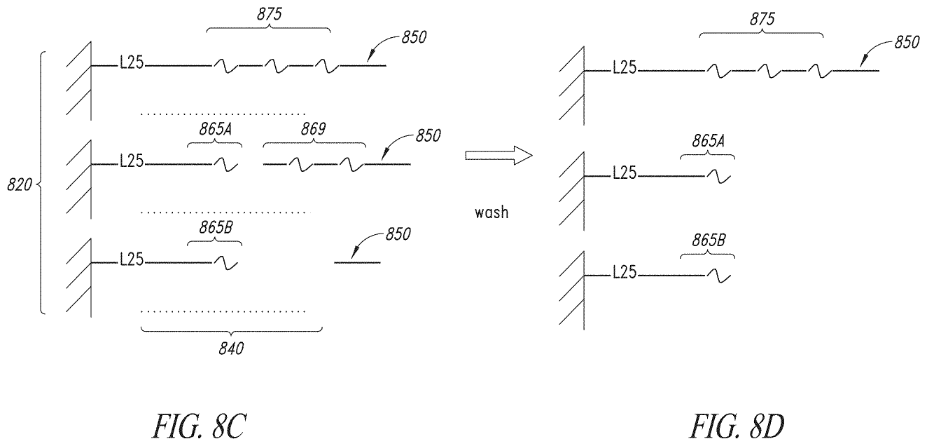

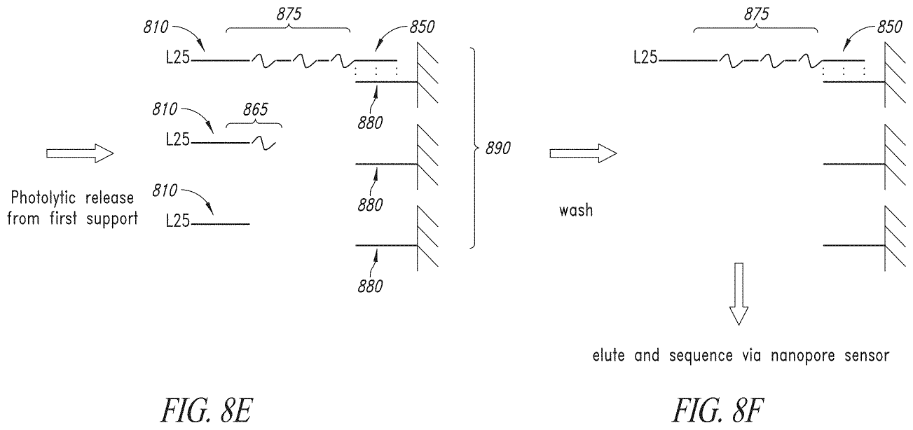

In this embodiment, Xpandomer synthesis is initiated from a primer-template complex bound to a support and extends away from the support towards an end cap structure hybridized to the opposite (i.e., 3′) end of the template. The initial configuration of the AFS model is depicted in FIG. 8A, with each of the three cartoons illustrating identical features. In this embodiment, the 5′ end of oligonucleotide primer 810 is bound to solid support 820 by linker 830. Single-stranded template 840 is hybridized to the primer via standard hydrogen bonding. Likewise, end-capped oligonucleotide 850 is hybridized to the 5′ end of the template via standard hydrogen bonding and provides a free 5′ triphosphate group 855. The directionality of nucleic acid polymerization (i.e., Xpandomer synthesis) is indicated by the arrow.

Exemplary products of an Xpandomer synthesis reaction initiating from primer 810 are illustrated in FIG. 8B. The top and middle cartoon depict full-length Xpandomer copy 870 covalently linked to primer 810 and hybridized to template 840 by hydrogen bonding. The full-length Xpandomer product is also covalently linked to the end-capped oligonucleotide 850 via a phosphodiester bond. The bottom cartoon depicts an incomplete Xpandomer copy 860 that remains covalently bound to the primer, but importantly, is not linked to the end capped oligonucleotide 850.

As discussed elsewhere herein, after synthesis, Xpandomers are processed and treated with acid to transition the Xpandomers from the constrained form depicted in FIG. 8B to the expanded, linearized form depicted in FIG. 8C. Here, template 840 is shown dissociated from the support-bound Xpandomers. The top cartoon shows linearized, full-length Xpandomer 875 still covalently bound to the solid-support 820 and the end capped oligonucleotide 850. The middle cartoon shows an alternative outcome to acid treatment, wherein the full-length Xpandomer has been cleaved to generate linearized fragments 865A and 869. Fragment 865A remains linked to the solid support while fragment 869 is released into solution from the support. The bottom cartoon shows linearized Xpandomer fragment 865B, also bound to the solid support. FIG. 8D illustrates that, after wash, full-length linearized Xpandomer 875 and linearized fragments 865A and 865B remain bound to the solid support. Importantly, only full length Xpandomer 875 is linked to the end capped oligonucleotide 850.

FIG. 8E illustrates how the end-capped oligonucleotide 850 can be used as a molecular tag to isolate, or “fish” out, full-length Xpandomer products from a heterogenous population including incomplete fragments. The Xpandomer products remaining bound to the initial support as illustrated in FIG. 8D are released from the support by photolysis. As described elsewhere herein, the linkage of the oligonucleotide primer to the initial solid support is designed to be light-sensitive. The released Xpandomers 865 and 875 remain covalently associated with the oligonucleotide primer 810 and full-length Xpandomer 875 remains covalently associated with the end capped oligonucleotide 850. To isolate full-length Xpandomers, the sample is contacted with a second solid support 890 that is conjugated with oligonucleotide 880, which is the reverse complement of the end capped oligonucleotide 850. As depicted in the figure, only full-length Xpandomer 875 will bind to the solid support via hydrogen bonding between oligonucleotides 850 and 880. As shown in FIG. 8F, all incomplete Xpandomer products can be washed away from the solid support, leaving isolated full-length Xpandomer 875, which can then be eluted from the support and used, e.g., for single-molecule nanopore sequencing. In this embodiment, the extension oligonucleotide includes features (e.g., the leader and concentrator elements) necessary for nanopore localization and translocation.

In an alternative embodiment, the end cap oligonucleotide is modified to include a leader and concentrator features for nanopore threading, while the extension oligonucleotide lacks these features. In this embodiment, only full-length extension products will be linked to the leader and concentrator elements and thus be capable of translocating through a nanopore to produce sequence information.

In another embodiment, the extension oligonucleotide structure is modified to include the leader and concentrator features for nanopore threading, while the end cap oligonucleotide lacks these features. In this embodiment, the Xpandomer synthesis and end-capping reactions may be conducted in-solution. Following Xpandomer synthesis, the end-capped products may be purified by contacting the sample with an oligonucleotide immobilized on a bead support, e.g., by biotin-streptavidin chemistry, in which the oligonucleotide includes a sequence that is the reverse complement of a portion of the sequence of the end cap oligonucleotide. In this manner, only those Xpandomer products that include both the extension oligonucleotide structure (providing the leader and concentrator features) and the end cap will thread through a nanopore sensor to provide sequence information.

Towards Support (TS) Xpandomer Synthesis Workflow

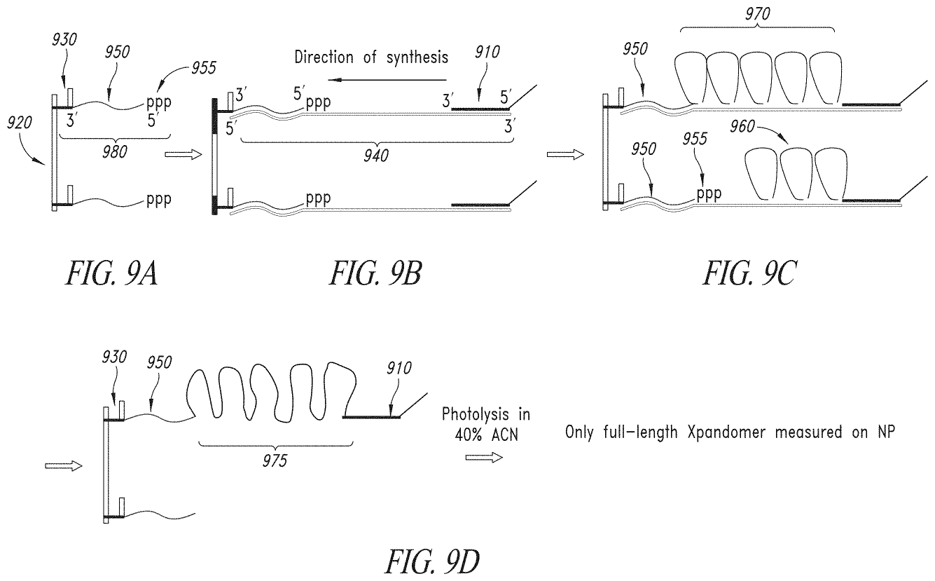

In an alternative embodiment of the invention, the terminal oligonucleotide of the end cap structure is covalently bound to the substrate. In this embodiment, Xpandomer synthesis is initiated from a primer-template complex that is hybridized to the terminal oligonucleotide of the end cap structure and the directionality of Xpandomer synthesis is towards the support. The initial configuration of the TS model is depicted in FIG. 9A, with each of the two support-bound end caps 980 illustrating identical features. In this embodiment, the 3′ end of the terminal oligonucleotide 950 is bound to solid support 920 by photocleavable linker 930. The end cap 980 provides free 5′ triphosphate 955.

The sequence of the terminal oligonucleotide of the end cap is designed to be the reverse complement of a sequence at the 5′ end of a single-stranded target nucleic acid template. FIG. 9B illustrates the association between the 5′ end of the target nucleic acid template 940 and the terminal oligonucleotide of the end cap via standard base-pairing. In this embodiment, extension oligonucleotide 910 is hybridized to a complementary sequence at the 3′ end of the template. Xpandomer synthesis initiates from the 3′ end of primer 910 and proceeds towards the support-bound end cap. The directionality of nucleic acid polymerization (i.e., Xpandomer synthesis) in this model is indicated by the arrow.

Exemplary products of an Xpandomer synthesis reaction initiating from primer 910 are illustrated in FIG. 9C. The top cartoon depicts full-length Xpandomer copy 970 covalently linked to primer 910 and end-capped oligonucleotide 950 via phosphodiester bonds. The bottom cartoon depicts an incomplete Xpandomer copy 960 that remains covalently bound to the primer, but importantly, is not linked to the terminal oligonucleotide 950 of the end cap.

As discussed elsewhere herein, after synthesis, Xpandomers are processed and treated with acid to transition the Xpandomers from the constrained form depicted in FIG. 9C to the expanded, linearized form as depicted in FIG. 9D. Here, template 840 and incomplete Xpandomer 960 have dissociated from the support and washed away from the bound material. The top cartoon shows linearized, full-length Xpandomer 975 covalently bound to the solid-support 920 by the terminal oligonucleotide 950 of the end cap. Importantly, only full-length Xpandomer copies remain bound to the solid support. These can be subsequently released by light-mediated cleavage of the photocleavable moiety 930 and used for nanopore sequencing.

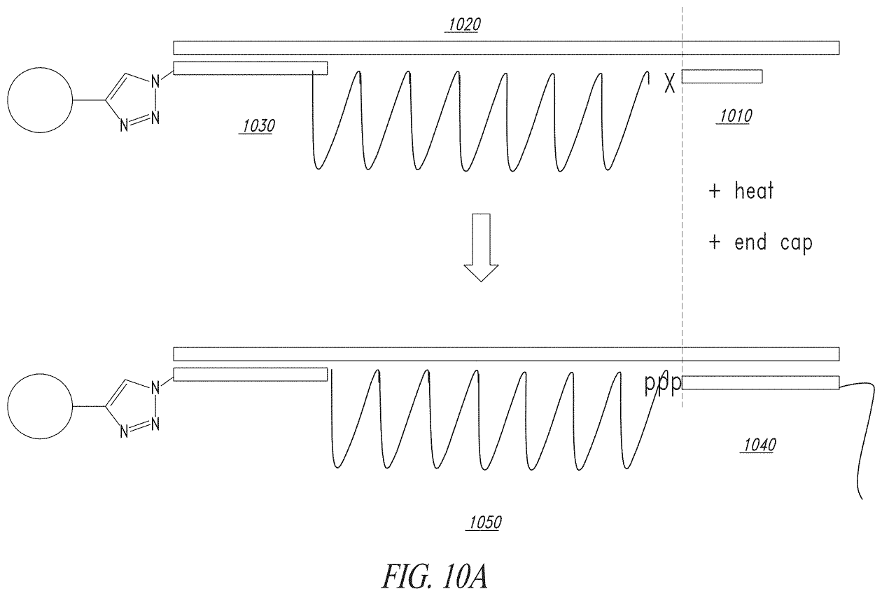

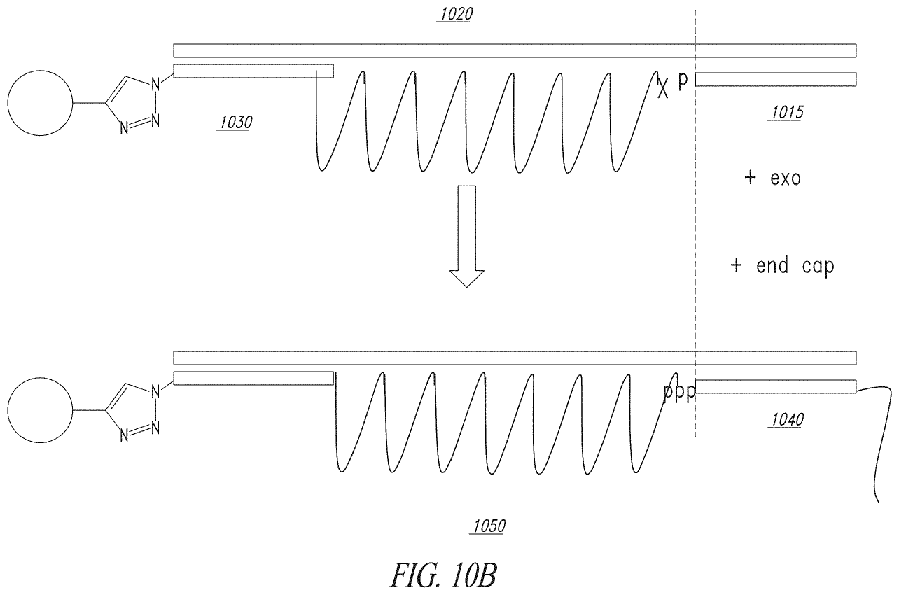

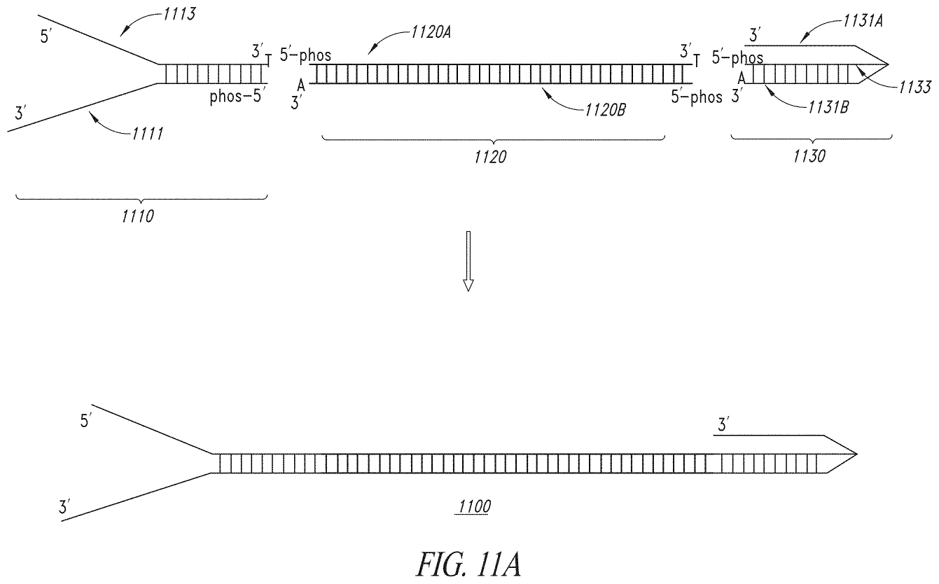

In some circumstances, truncated by-products may form during the end-capping process, e.g., if the DNA polymerase prematurely joins the end cap structure to an incomplete copy of the template. This phenomenon is referred to herein as polymerase “short-circuiting”. To prevent short-circuiting, the inventors have devised several strategies to delay incorporation of the end cap structure into the Xpandomer, thereby favoring synthesis of substantially full-length copies of the template. In one embodiment, outlined in FIG. 10A, blocker nucleotide 1010 is hybridized to a region near the 3′ end of single-stranded template 1020. The blocker oligonucleotide is designed to prevent incorporation into the growing Xpandomer by the DNA polymerase. In some embodiments, the 5′ end of the blocker oligonucleotide lacks a 5′ triphosphate group and is thus incapable of being joined to the 3′ end of the Xpandomer. Extension of oligonucleotide 1030 is thus stalled when the polymerase reaches the blocker oligonucleotide. At this point, the blocker oligonucleotide can be removed from the template, e.g. by thermal melting, and replaced by end cap oligonucleotide 1040, which is capable of being joined to substantially full-length Xpandomer 1050 by the DNA polymerase. Suitable melting temperatures can be calculated that result in dissociation of the short blocker oligonucleotide while not affecting hybridization of the longer Xpandomer with the template.

In another embodiment, as illustrated in FIG. 10B, blocker oligonucleotide 1015 is designed to provide a 5′ phosphate group. As discussed above, the DNA polymerase is incapable of incorporating the blocker oligonucleotide into the growing Xpandomer and synthesis is thus stalled when the polymerase encounters the blocker. In this embodiment, the blocker can be removed, e.g. by exonuclease-mediated digestion. Following exonuclease treatment, end cap oligonucleotide 1040 is hybridized to the template and joined to the substantially full-length Xpandomer 1050 by the DNA polymerase.

C. Libraries of Mirrored Xpandomers Constructed with End-Capping

This generalized embodiment describes novel methods and nucleic acid compositions that can be used to generate a library of template constructs in which each individual construct incorporates two single-stranded copies of the same strand of a nucleic acid target sequence (i.e., a template), joined in tandem by an oligonucleotide-based linker. A library of such template constructs is referred to herein as a “mirrored library”. The mirrored library provides the templates for a novel Xpandomer synthesis protocol that employs the end-capping strategy disclosed herein. Briefly, a single Xpandomer polymer is synthesized off each template construct, producing an Xpandomer product that includes the two copies of the same strand of a target that are operably linked by covalent bonding to a cap brancher structure. The two copies of the target sequence are each joined to the cap brancher structure during synthesis via the end-capping methodology described herein. Advantageously, Xpandomers synthesized from mirrored library constructs provide two sequence reads of a single target sequence when passed through a nanopore. Discrepancies between the sequences of the first and second reads indicate a potential sequencing error and can be excluded or subjected to quality scoring or some method of discrepancy resolution.

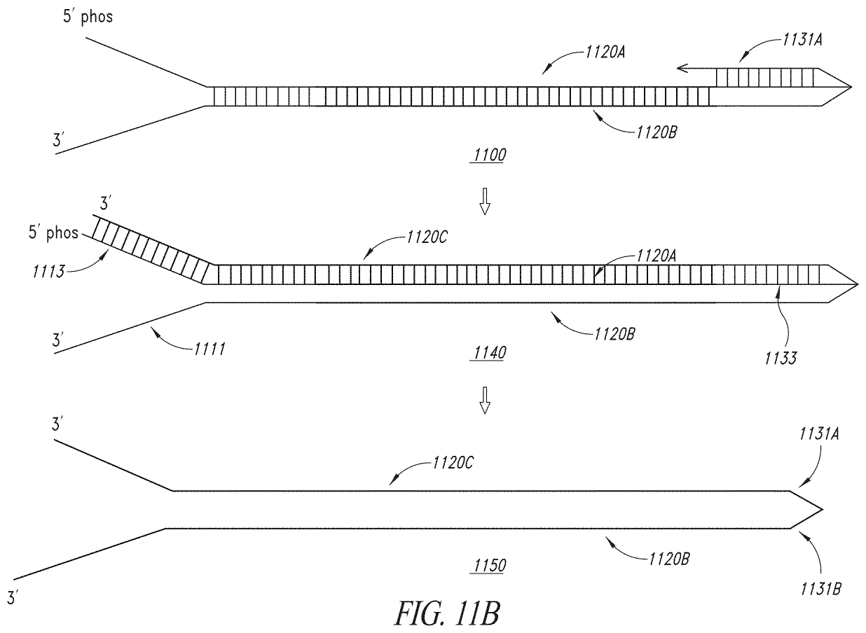

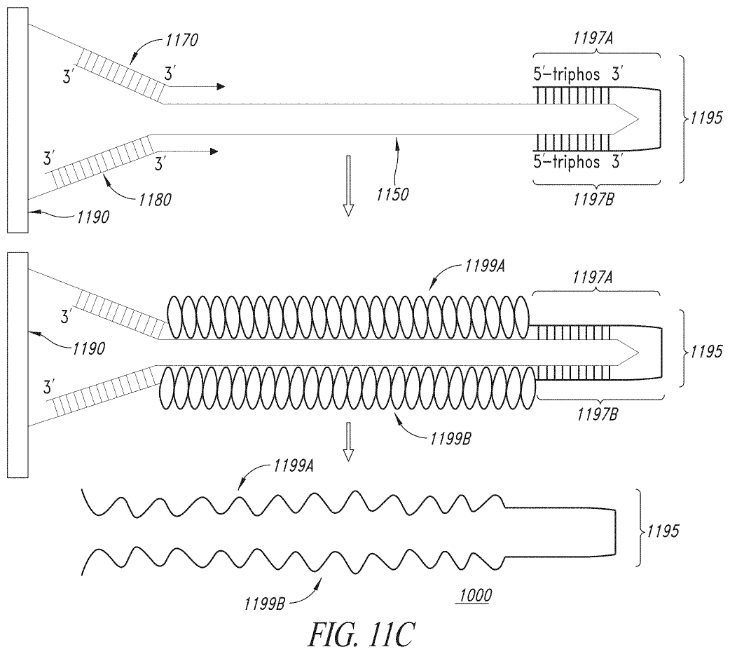

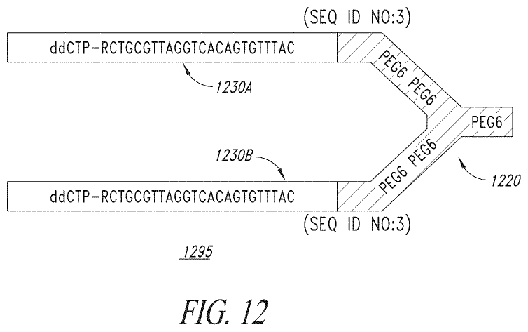

Mirrored library template constructs are produced through an ordered series of enzymatic reactions that each generates a characteristic precursor construct. FIG. 11A illustrates the basic structural features of one embodiment of a mirrored library template construct precursor, termed “M1”, 1100. The M1 precursor is formed by operable linkage (i.e., by joining or attaching via formation of covalent bonds) of Y adaptor construct 1110, library fragment 1120, and cap primer adaptor construct (referred to herein as the “trident”) 1130. In this embodiment, Y adaptor 1110 includes a 3′ to 5′ oligonucleotide strand 1111 and a 5′ to 3′ oligonucleotide strand 1113, herein referred to by convention as the “minus” and “plus” strands, respectively. The adaptor strands 1111 and 1113 specifically hybridize in the “stem” portion of the Y adaptor, proximal to the library fragment, while the “arm” portions, distal to the library fragment, remain single-stranded. The double-stranded stem portion of the Y adaptor can be joined to the library fragment. In this embodiment, the 3′ end of adaptor strand 1113 has an unpaired nucleotide, represented here by the free “T”, that can base pair with a free nucleotide provided by the library fragment to facilitate linkage. The arms of the Y adaptor can be engineered to provide several useful features for mirrored library workflow, including binding sites for oligonucleotide primers (i.e., extension oligos) used during the later stages of Xpandomer synthesis. In some embodiments, the ends of one or both singled-stranded regions of the Y adaptor strands provide an azide group that enables immobilization of the Y adaptor to a functionalized solid-support via a click reaction, as described herein. In other embodiments, one or both strands of the Y adaptor may include a selectively cleavable element that enables, e.g., release of the construct from a solid support. In some embodiments, minus strand 1111 is joined to a solid support while plus strand 1113 provides a 5′ nucleotide substrate for exonuclease digestion, as described further herein.

Library fragment 1120 is a double-stranded nucleic acid with, in one embodiment, 5′ phosphate termini and 3′ nucleotide overhangs on both strands that may be generated by art-recognized techniques. The library fragment is also referred to herein as the “nucleic acid target sequence” and is the target of sequence determination by SBX. The library fragment includes “plus” strand 1120A and “minus” strand 1120B. In some embodiments, the 3′ end of the minus strand may provide an unpaired nucleotide (represented here by the free “A”) that forms a base pair with the unpaired nucleotide at the 3′ end of adaptor strand 1113. In other embodiments, the 3′ end of the plus strand also provides an unpaired nucleotide (represented here by the free “T”) to facilitate linkage to cap primer adaptor 1130. The library fragment may include a known or an unknown sequence. For SBX, the length of the library fragment may be up to around 50, 100, 200, 500 or 1000 base pairs. In some embodiments, the length of the library fragment is from around 100 to around 200 base pairs.

Cap primer adaptor construct 1130 includes three oligonucleotide strands, 1131A, 1133, and 1131B, operably linked by a chemical brancher. The sequences of strands 1131B and 1133 are complementary and may hybridize. The sequence of strand 1131A is identical to 1131B and this strand may remain single-stranded in the cap primer adaptor 1130 (or in some instances may hybridize to strand 1133). In some embodiments, the 3′ end of strand 1131B provides an unpaired nucleotide (represented here by the free “A”) that forms a base pair with an unpaired nucleotide at the 3′ end of plus strand 1120A of the library fragment.

Cap primer adaptors may be produced by standard automated phosphoramidite-based oligonucleotide synthesis. In some embodiments, strand 1133 is first synthesized in the 5′ to 3′ direction followed by incorporation of a symmetrical chemical brancher (e.g., Chemgenes CLP-5215) that enables simultaneous 5′ to 3′ synthesis of strands 1131A and 1131B. In some embodiments, incorporation of standard hydrophilic spacers (e.g., PEG6 spacers) between the brancher and the 5′ ends of strands 1131A and 1131B provides flexible linkers that enable these strands to fold back on strand 1133 to form the characteristic “trident” structure of the cap primer adaptor. The length and composition of both the oligonucleotide and brancher constituents of the cap primer adaptor can be optimized to for particular applications. In certain embodiments, the oligonucleotides are around 15 to 25 nucleotides in length and enable efficient hybridization with the cap brancher construct as discussed below.

The mirrored library template constructs may be formed in-solution or on a solid support. In one embodiment, mirrored library template constructs are formed on a solid support by first producing the M1 precursor according to the following exemplary steps: 1) Y adaptor strand 1111 is immobilized on a functionalized solid-support (e.g., a microfluidic chip or bead) via a click reaction and the Y adaptor strand 1113 is then specifically hybridized to adaptor strand 1111; 2) The cap primer adaptor 1130 is attached to library fragment 1120 via in-solution enzymatic ligation of the 3′ end of the plus strand 1120A to the 5′ end of strand 1133 and ligation of the 5′ end of the minus strand of 1120B to the 3′ end of fragment 1131A; and 3) The ligated library fragment-cap primer adaptor structure is then attached to the support by enzymatic ligation to the end of the double-stranded portion of the Y adaptor 1110.

The M1 mirrored library template construct precursor 1100 provides the substrate for the formation of the final mirrored library template construct, termed “M3”, 1150 depicted in FIG. 11B. In one embodiment, template construct 1150 may be produced by two enzymatic steps: a first DNA polymerization step that produces a complement of plus strand 1120A, followed by a second exonuclease step that removes this same plus strand. During the first step, cap primer adaptor strand 1131A is extended by a DNA polymerase, e.g., a strand-displacing, thermostable polymerase, from the 3′ end in the direction indicated by the arrow using strand 1120A as a template; this produces a three-stranded structure, herein referred to as template construct precursor “M2” 1140. The M2 precursor includes daughter strand 1120C with the same sequence as minus strand 1120B. During the second step, the middle oligonucleotide strand of the M2 precursor is enzymatically removed by exonuclease digestion initiating from the 5′ end of Y adaptor strand 1113, which provides a 5′ phosphate substrate for the exonuclease. The entire original plus strand 1120A is thus removed, as is cap primer adaptor strand 1133. The resulting product is mirrored library template construct “M3” 1150 that includes two identical copies, 1120B and 1120C, of the original minus strand of the library fragment joined by strands 1131A and 1131B of the cap primer adaptor, which remain joined together. The M3 mirrored library construct 1150 may be used as a template to synthesize a single Xpandomer that includes two copies of the same strand of library fragment 1120.

As discussed herein, the M3 constructs function as templates for the synthesis of Xpandomers that each contain two copies of the same strand of a target sequence for nanopore sequencing, i.e., sequencing by expansion (SBX). In some embodiments, SBX of mirrored library constructs is conducted on a solid-support and employs the end-capping protocol described herein. In this embodiment, depicted in FIG. 11C, the 5′ ends of extension oligonucleotides 1170 and 1180 are linked to a solid support 1190 by click chemistry, as described herein. In these embodiments, the extension oligonucleotides include 5′ azide groups to mediate click attachment. In other embodiments, only one extension oligonucleotide is linked to the support, while the other extension oligonucleotide includes a leader sequence for threading through a nanopore. Each extension oligonucleotides is designed to specifically hybridize with one of the single-stranded portions of the Y adaptor element of the M3 template construct. In certain embodiments the extension oligonucleotides may include a photocleavable element or an acid cleavable element interposed between the solid support and the 5′ end of the oligonucleotide sequence to enable light or acid-mediated release of the final Xpandomer product from the substrate. The M3 template construct 1150 is hybridized to the immobilized extension oligonucleotides 1170 and 1180 via standard hybridization between the complementary sequences in the extension oligonucleotides and the arms of the Y adaptor portion of the M3 construct. A cap brancher construct 1195 is hybridized to the M3 construct. The cap brancher 1195 includes two identical oligonucleotides 1197A and 1197B, which are complementary to, and hybridize with, the 5′ ends of both strands of the mirrored library construct 1150. The terminal oligonucleotide arms 1197A and 1197B each provide free 5′ triphosphate groups. The cap brancher structure may be synthesized by conventional phosphoramidite chemistry in which the two strands 1197A and 1197B are joined by a chemical brancher.

FIG. 12 illustrates further details of the structural features of the cap brancher. In this embodiment, cap brancher 1295 includes brancher structure 1220, terminal oligonucleotide arms 1230A and 1230B, which include triazole moieties (“R”), end caps (“ddCTP”), and an oligonucleotide (SEQ ID NO:3). The cap brancher is synthesized by standard phosphoramidite chemistry initiating from a 3′ terminal moiety, herein exemplified by a PEG6 polymer. A symmetrical chemical brancher is added to the 5′ end of the terminal moiety to enable parallel synthesis of brancher spacers, herein exemplified by PEG6 polymers. In some embodiments, the length and composition of the spacers can be optimized for particular applications. In certain embodiments, spacers may include monomers of C2, C6, or PEG3. Terminal oligonucleotide arms 1230A and 1230B extend off the 5′ end of the brancher arms. The sequences of the terminal oligonucleotides are designed to hybridize to the 5′ ends of the M3 template construct, the sequences of which are provided by the cap primer adaptor. In some embodiments, the terminal oligonucleotides are from around 15 to around 50 nucleotides in length and include one or more methoxy nucleotide analogs. The 5′ ends of the terminal oligonucleotides are joined to end cap structures, herein exemplified by ddCTP (although any of the other nucleobases could be substituted in certain embodiments), that enable attachment of nascent Xpandomers to the terminal oligonucleotides via end-capping. Details of the end-capping methodology are discussed herein and with reference to FIGS. 7A-7D. The end caps are joined to the terminal oligonucleotides via triazole moieties (“R”), which are the products of click reactions between an alkyne moiety provided by the end cap and an azide moiety provided by terminal oligonucleotide. In some embodiments, the cap brancher is designed to include other linker structures, e.g., spermine polymers positioned between the end cap and the terminal oligonucleotides to provide, e.g., increased steric flexibility and binding to the end caps.

With continued reference to FIG. 11C, Xpandomer synthesis reactions are conducted, which initiate at the 3′ ends of extension oligonucleotides 1170 and 1180, proceed in the same direction (as indicated by the arrows) and terminate at the 5′ ends of terminal oligonucleotides 1197A and 1197B of the cap brancher 1195, upon which the polymerase joins the complete Xpandomer copies 1199A and 1199B to the cap brancher according to the end-capping methodology described herein. In one embodiment, a first extension oligonucleotide includes a photocleavable linker element and a second extension oligonucleotide includes an acid-labile linker element. Acid treatment of the Xpandomer will simultaneously transition the Xpandomer copies from the “constrained” to the “open” configuration 1000 and cleave the acid-labile linker in the extension oligonucleotide. The resulting product including two joined Xpandomers 1199A and 1199B of the library fragment can then be removed from the support by photolysis of the photocleavable linker of the second extension oligonucleotide. In some embodiments, a final purification step is performed in which the released mirrored Xpandomer 1000 is hybridized to an oligonucleotide complementary to one of the extension oligonucleotide attached to a second solid support.

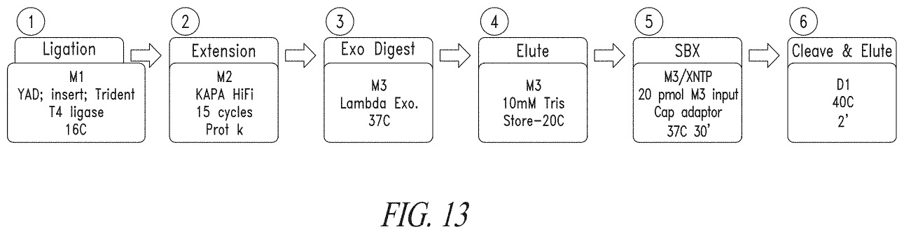

Reaction conditions for the production of the M1, M2 and M3 mirrored library constructs and SBX to synthesize Xpandomers can be optimized through trial and error. In some embodiments, these constructs may be produced by the following the workflow outlined in FIG. 13. In step 1, the M1 precursor is produced through ligation of the Y adaptor, the library insert, and the Trident. The molar ratios of YAD1:YAD2:insert:Trident and can be optimized for specific conditions or applications. In some embodiments, the M1 precursor may be produced on a microfluidic chip by first assembling the Y adaptor on an alkyne-functionalized chip. In one embodiment, a first Y adaptor strand providing a terminal azide group is attached to the functionalized chip by click chemistry according to the following exemplary protocol: 1) a catalyst mix is prepared including 3.0 mM THPTA, 6.0 mM sodium ascorbate, 1 mM CuSO₄, 5.0 mM aminoguanidine, and 10% DMF or DMSO and a substrate mix is prepared including 10% DMF or DMSO, 25 mM sodium phosphate, pH 7.0, 1 μM azide-Y adaptor oligonucleotide strand 1, 2.5 mM MgCl₂, 5 mM amino guanidine, and 6.0 mM sodium ascorbate; 2) 11 μl of the catalyst mix is added to 44 μl of the substrate mix and 50 μl of this reaction is added to an alkyne-functionalized microfluidic chip, such as a COC chip, and incubated for 20′ at room temperature; 3) the chip is washed with 300 μl of solution 10002 (0.3M sodium phosphate, pH8.0, 1% Tween 20, 0.5% SDS, and 1 mM EDTA) for 5′ at 37° C. then washed with 900 μl of buffer A.1 (0.5M NH₄OAc, pH 6.5, 1M urea, 5% NMS, and 2% PEG8000). Following the click attachment, a second Y adaptor strand is hybridized to the substrate-bound first strand by preparing a hybridization mix including 100 pmol of the second oligonucleotide in buffer A.1. The hybridization mix is incubated at 90° C. for 15″ then cooled to 72° C. The mix is then added to the pre-heated chip and the chip is allowed to cool to 32° C. for 5′ using a thermocycler. The chip is then washed with buffer A.1. Next, the library insert and Trident adaptor are ligated to the bound Y adaptor. The insert fragment is denatured for 3′ at 90° C. in a buffer including 100 mM NaCl/20 mM Tris, pH 8.0 then ramped down to 50° C. over 5′ using a thermocycler. A ligation mix is prepared including 20 pmol doubled-stranded insert, 50 pmol Trident adaptor, 3 mM ATP, 2 U/μl T4 PNK, and 200 U/μl T4 DNA ligase in 1× ligation buffer (66 mM Tris, 10 mM MgCl2, 1 mM DTT, and 7.5% PEG6000). The ligation reaction is run for 15′ at 16° C. then the reaction is added to the chip to which the Y adaptor is bound. The chip is incubated for 15′ at 16° C. The ligation mix is then removed and 3 μl of 5′ deadenylase (50,000 U/ml) is added to the ligation mix, and the ligation mix is added back to the chip and the chip is incubated for 15′ at 16° C. The chip is then washed with 4 ml of buffer 10002 for 5′ at 37° C. The chip is next washed with water and can be stored at 4° C. in 10 mM Tris.

In step 2, the M2 precursor is prepared by extension of the M1 precursor. In one embodiment, approximately 2.5-10 pmol of chip-bound M1 is used in an extension reaction including 1.0× polymerase buffer, 0.2 mM each dNTP, 0.28 U/μl DNA polymerase and 1 mM MgCl₂. Suitable DNA polymerases are Vent (exo-) DNA Polymerase or KAPA HiFi. The chip is placed in a thermocycler and incubated 1′ at 95° C. followed by from 10 to 40 cycles of 20″ at from 90 to 98° C. followed by 6″ at 76° C. The chip is washed with water to remove excess reagents. The chip is then treated with proteinase K by adding a solution containing from 0.05 U/μl to 0.80 U/μl of proteinase K in water and incubating 5′ at 55° C. followed by 5′ at 95° C. The chip is washed with water.

In step 3, the M3 template construct is produced by exonuclease digestion. In some embodiments, an exonuclease digestion mixture including 0.45 U/μl lambda Exonuclease in exonuclease buffer is added to the chip and incubated for 5′ at 37° C. followed by 10′ at 75° C. The chip is washed with buffer 10002 followed by water then stored in a buffer containing 10 mM Tris.

In step 4, the bound M3 construct is released by photocleavage. In some embodiments, the chip is exposed to UV light (e.g., 365 nm) for 15″ via a UV curing lamp (e.g., a Phoseon Technology FireFly lamp). The released M3 construct is recovered by aspirating the liquid off the chip.

In step 5, Xpandomer copies of the M3 template constructs are produced by the SBX methodology. In some embodiments, Xpandomers are produced on a microfluidic chip to which a first extension oligonucleotide (e.g., a “E52” EO) is covalently bound via click chemistry as described in step 1. This RD may be referred to herein as the “capture oligo”. The capture oligonucleotide is used to assemble the M3 template, a second extension oligonucleotide, and a cap brancher structure on the chip by hybridization. The capture chip is washed with buffer A1 (0.5M NH₄OAc, pH 6.5, 1M urea, 5% NMS, and 2% PEG8000) and incubated at 65° C. A hybridization mix is prepared containing from around 5 pmol to around 30 pmol M3 construct, from around 20 pmol to around 80 pmol of the second extension oligonucleotide (e.g., a “E6 EO; the actual amount will be determined by the amount E52 capture oligo bound to the chip) and from around 20 pmol to 80 pmol cap brancher (the actual amount will be the around the same as the amount of EO). The hybridization mix is incubated at 95° C. for 15” then added to the chip and incubated at 65° C. for 30″ and ramped down to 37° C. at a rate of 0.1° C./sec and held here for 5′. Chip incubation temperature is controlled by a standard thermocycler fitted with an in situ hybridization adapter plate.

For Xpandomer synthesis, an extension mix is prepared by mixing Buffer P (0.6 mM MnCl₂ and 0.18 μg/μl DPO4 DNA polymerase variant) with Buffer X, (80 μM PP-60.22 and 80 μM each XNTP) followed by addition of Buffer A (50 mM Tris, pH 8.84, 200 mM NH₄OAc, pH 6.88, 20% PEG8K, 5% NMS, 0.2 μg/μl SSB, 0.5M betaine, 0.25M urea, 1 mM PEM AZ-8,8 and 4 mM PEM additive). The extension mix is added to the chip and incubated for 15′-60′ at 20-45° C. The chip is washed with Buffer B (100 mM HEPES, 100 mM NaHPO4, 5% Triton, and 10% DMF).

In step 6, the Xpandomer is cleaved and eluted in 0-75% ACN. In one embodiment, the capture oligonucleotide includes a photocleavable element. To release the Xpandomer from the chip, the chip is exposed to UV light for 15″. The chip is then incubated at 37° C. for 2′ and an Xpandomer sample is removed with a pipette.

For nanopore sequencing, one or both extension oligonucleotides include a leader sequence designed to promote threading of the Xpandomer through a nanopore. Further details of certain embodiments of leader sequences are disclosed in Applicants' issued U.S. Pat. No. 9,670,526 “Concentrating a Target Molecule for Sensing by a Nanopore”, which is herein incorporated by reference in its entirety. In one embodiment, the sequence of an exemplary extension oligonucleotide is represented by: RD₁₀(PC)L₂₅Z₆[TCATAAGACGAACGGA (SEQ ID NO:4)] in which “R” represents a 5′-azide group that enables attachment to a functionalized solid-substrate by click chemistry; “D” represents a poly-PEG6 spacer; “PC” represents a photocleavable spacer to enable release from the solid substrate; “L” represents a poly-C2 spacer that functions as a leader sequence during nanopore translocation; “Z” represents a poly-C12 spacer, and TCATAAGACGAACGGA (SEQ ID NO:4) represents an oligonucleotide that will hybridize to a target sequence and function as an extension primer for a DNA polymerase. In other embodiments, the PC spacer may be replaced by an acid labile spacer, e.g., a [dT p-ethoxy][DMS(O)MT-NH₂—C6 or glen amidite 10-1907] phosphoramidite. The number of each phosphoramidite monomer (i.e., “spacer”) designed into an extension oligonucleotide is variable and may be optimized for particular applications. During mirrored library synthesis, the leader sequence may be included in one or, in other embodiments, both of the extension oligonucleotides that initiate Xpandomer synthesis. In certain embodiments, the leader sequence is provided by a first extension oligonucleotide that is not covalently bound to the substrate, while a second extension oligonucleotide that is attached to the substrate lacks a leader sequence. Following Xpandomer synthesis and processing, any truncated products not attached to the second extension oligonucleotide can be removed from the substrate by washing. Following release of the Xpandomers from the substrate, any truncated products not attached to the first extension oligonucleotide will lack the leader sequence and, advantageously, fail to thread through the nanopore to provide sequence data.

D. Next-Generation, YAD-Free, Mirrored Library Constructs and Methods

Several features of the mirrored library workflow discussed herein are amenable to modification and/or optimization to provide advantages for particular experimental demands. In the embodiments illustrated in FIGS. 11A-11C, binding sites for the Xpandomer extension oligonucleotides and functional groups for solid-state attachment are provided by the Y adaptor, which is joined to the library fragment by enzymatic ligation. In an alternative, “next-generation” embodiment, binding sites for the extension oligonucleotides are, instead, provided by oligonucleotide primers that are joined to the library fragments via PCR. This approach enables both amplification of the target sequence and elimination of the ligation step that joins the YAD to the library fragment. Following incorporation of the primer sequence into the library fragment, the resulting PCR product is referred to as a “tailed” or “tagged” library fragment (or, alternatively, “tagged target sequence”). In some embodiments, functionalized end groups for solid-state attachment are provided by a separate oligonucleotide structure, that includes an oligonucleotide sequence referred to herein as the “capture oligo” that is designed to specifically hybridize with the library tag following PCR amplification. In general terms, these embodiments are referred to herein as “YAD-free” mirrored library construction.

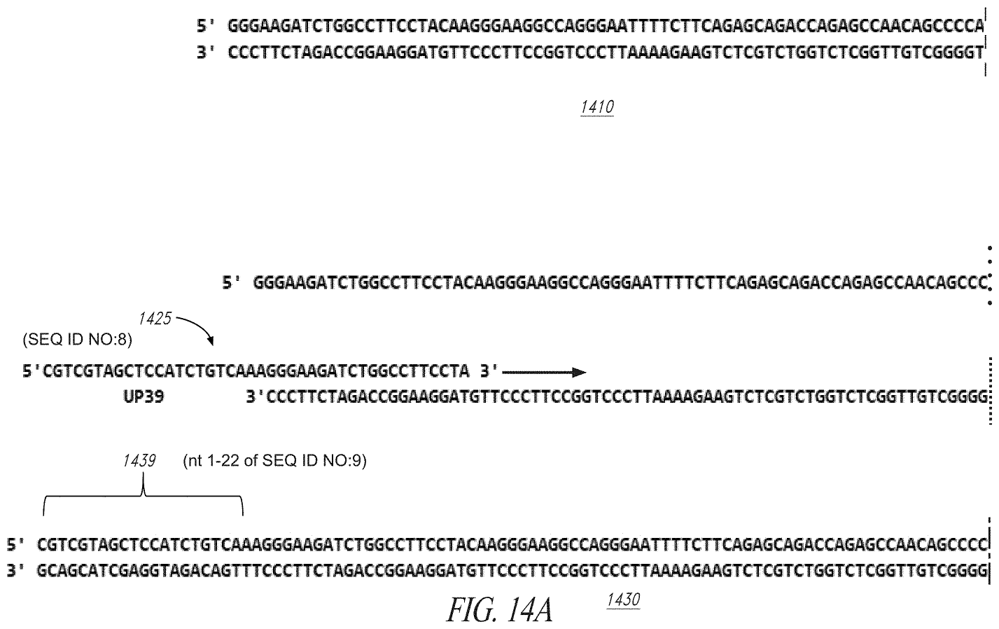

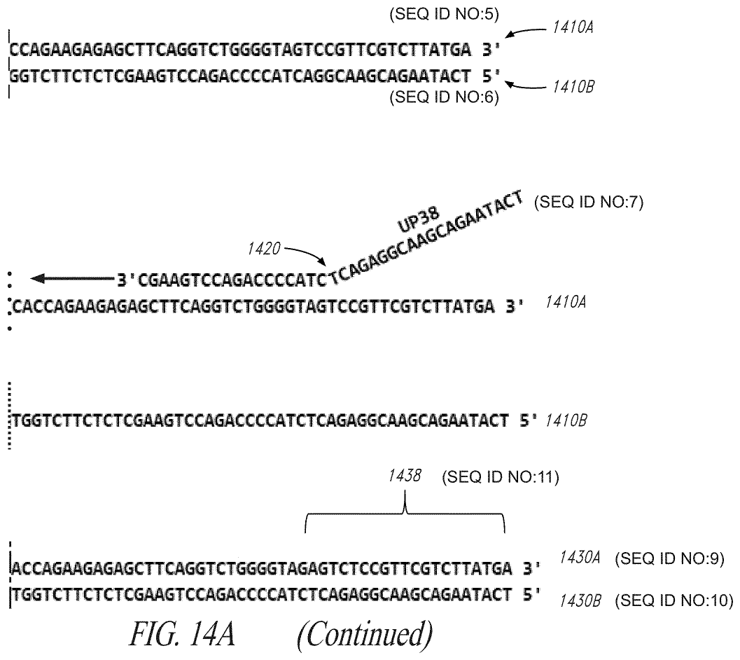

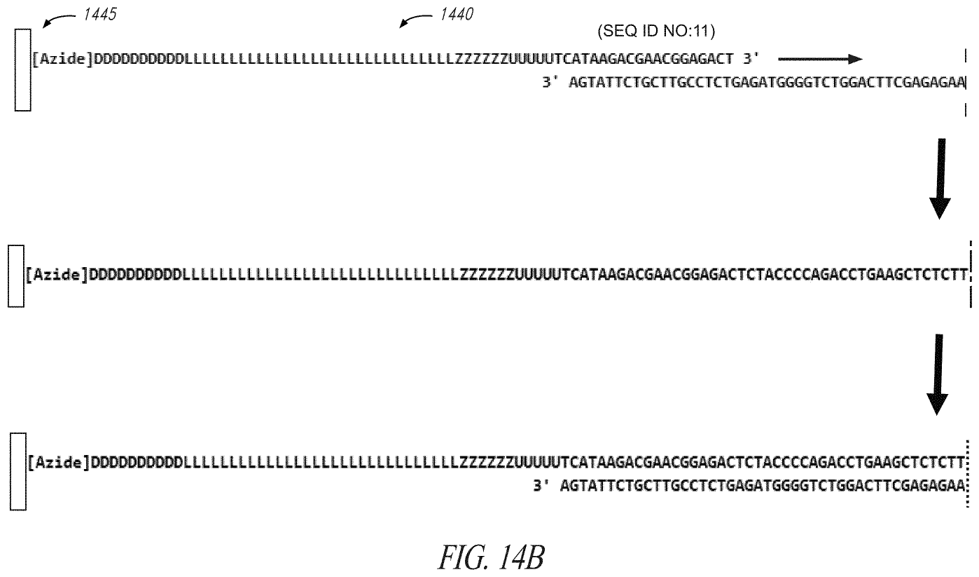

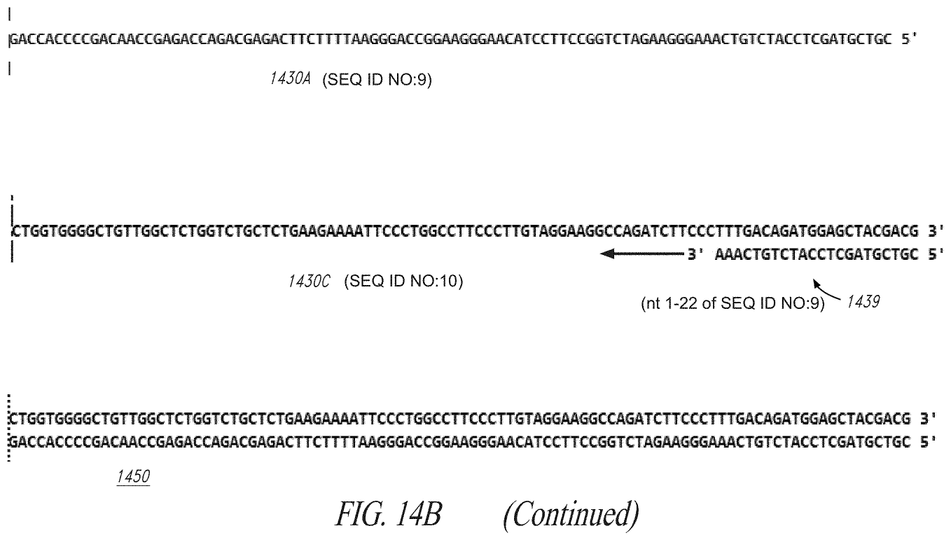

One embodiment of YAD-free tagging and capture of a library fragment, i.e. DNA target sequence, is illustrated in FIG. 14. In this embodiment, the library fragment is exemplified by double-stranded 100mer 1410 with plus strand (SEQ ID NO:5) 1410A and minus strand (SEQ ID NO:6) 1410B. Forward and reverse PCR primers are designed that include oligonucleotide sequences complementary to the target sequence linked to heterologous sequences at their 5′ ends. In one embodiment, primer (SEQ ID NO:7) 1420 includes a 3′ oligonucleotide sequence that specifically hybridizes to a complementary sequence in plus strand 1410A of the library fragment and a 5′ heterologous sequence that introduces a tag into the PCR product that enables capture of the tagged library fragment. In this embodiment, the 5′ heterologous sequence is referred to as “UP38” and is the same sequence that is present in both the capture oligonucleotide structure and the Xpandomer extension oligonucleotides. In some embodiments, primer (SEQ ID NO:8) 1425 includes a 3′ oligonucleotide sequence that specifically hybridizes with a complementary sequence in minus strand 1410B of the target sequence and a 5′ heterologous sequence that provides binding sites for the cap adaptor structure incorporated during Xpandomer synthesis. FIG. 14A shows the PCR primers hybridized to single-stranded plus strand 1410A (SEQ ID NO:5) and minus strand 1410B (SEQ ID NO:6). PCR amplification of the library fragment produces tagged fragment 1430 (with plus strand (SEQ ID NO:9) 1430A and minus strand (SEQ ID NO:10) 1430B) that includes first tag (SEQ ID NO:11) 1438 and second tag (nucleotides 1-22 of SEQ ID NO:9) 1439 whose sequences are determined by the heterologous sequence tails of the PCR primers. Standard primer design principals, which are well established in the art, are followed when designing primers 1420 and 1425.

For capture of the tagged library fragment, a capture oligonucleotide structure is covalently linked to solid-support via, e.g., click chemistry as described herein. One embodiment of a generalized capture oligonucleotide structure may be represented as follow: [azide]DₙLₙZₙ(SCL)(CO), wherein the azide provides means for covalent attachment (i.e., immobilization) to a functionalized solid support (e.g., functionalized with an azide group or a dual-biotin group); D represents PEG6, L represents C2, and Z represents C6, wherein polymers of D, L, and Z can form a flexible linker structure; (SLC) represents a selectively cleavable linker, which in this embodiment is a multimer of uracil residues; and (CO) represents the oligonucleotide sequence of the capture oligo. In this embodiment, the CO sequence is the same sequence as the UP38 heterologous sequence (SEQ ID NO:11) and will specifically hybridize to the tag sequence of the plus strand of the library fragment. In some embodiments, the flexible linker is formed solely from PEG6 monomers, e.g., D₁₆, which provides advantages when PCR reactions are conducted on beads or on a microfluidic chip, as discussed herein.

To capture the tagged library fragment, a second PCR reaction is conducted in which the second PCR reaction is conducted on a solid-support that provides the capture oligonucleotide. Capture of the library fragment is illustrated in simplified form in FIG. 14B. Here, capture oligonucleotide structure 1440 is immobilized on solid-support 1445. The capture oligonucleotide structure includes a 3′ oligonucleotide sequence identical to the sequence of tag (SEQ ID NO:11) 1438 in the minus strand 1430B of the library fragment. When the double-stranded library fragment is denatured, plus strand 1430A specifically hybridizes to the capture oligonucleotide. The capture oligonucleotide provides a primer for synthesis of a copy of the complement of plus strand 1430A, here represented by 1430C (SEQ ID NO:10). A suitable number of PCR cycles will produce doubled-stranded library fragment 1450 immobilized on the solid-support.

Reaction conditions for in-solution tagging of library fragments followed by on-chip capture of tagged amplicon products can be optimized through trial and error. In one embodiment, the in-solution PCR tagging reaction may be run as follows: a reaction mix is prepared that includes 1-15 amol synthetic template DNA (or, in other embodiments, sheared natural library DNA), 2 μM each primer, 350 μM dNTPs, 1×KOD buffer (120 mM Tris, pH 8.0, 20 mM KCl, 6 mM NH₄SO₄, 1.5 mM MgSO₄, and 1% Triton X100), 0.05 U/μl KOD polymerase; the reaction is cycled at 95° C. for 2′ followed by 30 cycles of 95° C. for 10″/68° C. for 8″/72° C. for 8″, and a single 3′ extension at 72° C.; a final yield of ˜25 pmol tagged amplicon may be purified by, e.g., a QIAquick column (available from QIAGEN).

In one embodiment, a capture chip may be prepared as follows: 100 pmol of UP38 capture oligonucleotide is covalently attached to an alkyne-functionalized chip by a click reaction that includes 10% DMF, 3 mM THPTA, 25 mM Na₃PO₄, 5 mM aminoguanidine, 6 mM NaAsc, and 1 mM CuSO₄; the reaction is run for 20′ at room temperature then the chip is washed followed by BSA passivation (10 mg/ml non-acetylated BSA in PBS for ˜1 hour at room temperature).

In one embodiment, an on-chip PCR reaction may be run as follows: ˜1×10⁶ copies of the tagged amplicon product, 200 pmol UP39 primer, and 5 pmol UP38 primer are added to the chip containing ˜100 pmol bound UP38 capture oligonucleotide; a PCR mix is added that includes KAPA HiFi HS U+, 1× ReadyMix buffer (2.5 mM Mg), 0.1 μg/ml non-acetylated BSA, 1M betaine, 2% DMSO, 1% PEG and 0.5% Tween; the PCR cycling conditions are as follows: 2′ at 98° C., 35 cycles of 100° C. for 1′/48° C. for 12″/67° C. for 30″/80° C. for 2′ followed by a final 2′ at 80° C.; the chip is then washed in a buffer containing 1M NaCl and 10 mM Tris, pH 8.0.

The tagged library fragments captured on solid-support provide the substrates for production of the M3 mirrored library template constructs, which provide the templates for Xpandomer synthesis, as discussed herein. Several alternative workflows for M3 and Xpandomer production are contemplated by the present invention. What follows is a non-limiting discussion of certain embodiments of alternative “next generation” mirrored library workflow.

Single-support mirrored library production utilizing bystander extension oligonucleotides.

In this embodiment, both the M3 mirrored library template construct and the Xpandomer are synthesized on the same solid-support, e.g., a bead or microfluidic chip. Both the capture oligonucleotide for M3 production and the extension oligonucleotides for Xpandomer synthesis are immobilized on the support. In some embodiments, the extension oligonucleotides are designed to form a hairpin structure that prevents hybridization with the library fragment during PCR-based capture, and are thus referred to herein as “bystander” oligonucleotides. The bystander oligonucleotides may be selectively converted into functional extension oligonucleotides following capture of the tagged library fragment, as discussed further below.

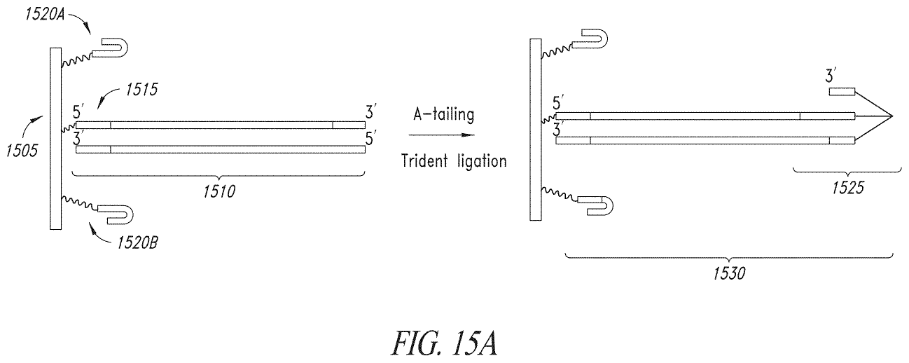

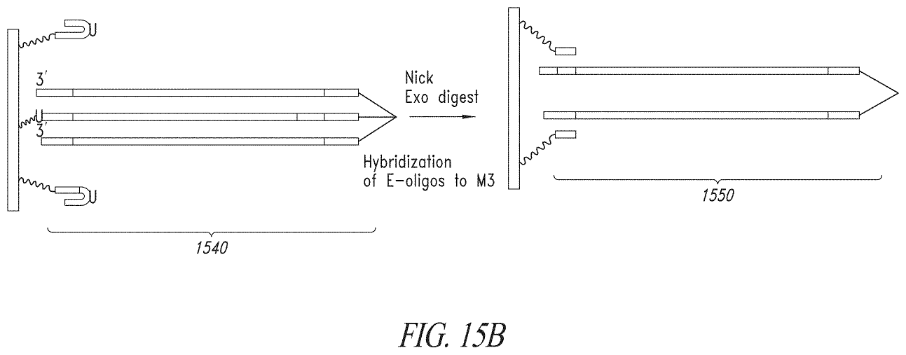

FIGS. 15A and 15B illustrate the basic features of single-support synthesis with bystander extension oligonucleotides. In FIG. 15A, tagged library fragment 1510 is shown immobilized on solid-support 1505. PCR-based tagging of the library fragment and linkage to the solid-support by capture oligonucleotide structure 1515 are carried-out as described herein and with reference to FIG. 14. In one embodiment, the capture oligonucleotide structure may have the following sequence: 5′ [azide]D₁₆(UUUUU)(UP38) 3′, in which the azide group mediates attachment to the solid-support, “D” represents a PEG6 linker, “U” represent deoxy uracil, and “UP38” represents the capture oligonucleotide sequence. The U₅ sequence is selectively cleavable, e.g., by USER® (Uracil-Specific Excision Reagent), available from NEB, which generates a single nucleotide gap at the location of a uracil residue and cleaves the resulting abasic site. The bystander extension oligonucleotides 1520A and 1520B are also immobilized on the support. The sequence of the bystander oligonucleotides are designed to form a double-stranded hairpin structure that prevents hybridization with the library fragment during PCR. In one embodiment, the bystander oligonucleotide may have the following sequence: 5′ [azide]DₙLₙZₙ[CATAAGACGAACGGAGAUUTCCGTTCG (SEQ ID NO:12)]X 3′, in which the “D”, “L”, and “Z” moieties form polymers that perform specific functions during SBX, as discussed further herein, while the 3′ terminal TCCGTTCG sequence folds back to base pair with the internal CGAACGGA sequence, thus forming a hairpin structure in which the intervening GAUU sequence remains single-stranded. The single-stranded uracil-containing sequence can be cleaved with USER®. The terminal “X” moiety of the bystander oligonucleotide represents a “blocker” (e.g., a PEG or C3 spacer blocker) that prevents extension from the oligonucleotide during PCR.

To form M1 precursor construct 1530, trident adaptor 1525 is ligated to the immobilized library fragment. In some embodiments, this may be accomplished by first adding an “A” tail to the free 3′ end of the library fragment, which forms a base pairs with a free 3′ “T” provided by the Trident construct. An exemplary A-tailing reaction may include 10 pmol PCR amplicon, 1× MolTaq buffer, 1 mM dATP, and 2.5 U MolTaq and run for 30′ at 72° C. An exemplary ligation reaction may include 40 pmol trident construct, 1× ligation buffer, 3 mM ATP, 2 U/μl T4 PNK, and 30 U/μl T4 DNA ligase and run for 20′ at room temperature, followed by addition of 150 U of 5′ deadenylase and incubation for 10′. The M1 precursor is then extended to form the triple-stranded M2 construct with a DNA polymerase, as described herein, and with reference to FIG. 11B.

FIG. 15B shows the M2 precursor construct 1540 with the selectively cleavable uracil moieties in the bystander extension oligonucleotides and the capture oligonucleotide designated by the letter “U”. To generate the M3 template construct 1550, the M2 precursor is subjected to cleavage is with USER® to nick the uracil moieties. This results in 1) cleavage of the hairpin structure in the extension oligonucleotides and 2) cleavage of the capture oligonucleotide to produce a free 5′ end in the middle strand of the M2 construct. At the same time, the M2 precursor is subjected to exonuclease treatment that 1) digests the terminal TCCGTTGC sequences of the bystander oligonucleotides to expose the extension oligonucleotide sequences, and 2) digests the middle strand of the M2 complex from the 5′ to 3′ end. The exposed extension oligonucleotides then specifically hybridize with the complementary sequences provided by the 3′ ends of the M3 template construct. In some embodiments, the nicking and exonuclease digestion reactions may be carried out by treating the M2 precursor with a reaction mix including 1× lambda exo buffer (67 mM glycine-KOH, 2.5 mM MgCl2 and 50 μg/ml BSA), 20% PEG8000, 0.15 U/μl USER®, and 0.4 U/μl Lambda exonuclease for 15′ at 37° C. Following the nicking and exonuclease digestion reactions, a subsequent phosphatase reaction is performed to remove the 3′ phosphate left by the USER® cleavage of the bystander oligonucleotide to make it a functional extension oligonucleotide for Xpandomer synthesis. In some embodiments, the phosphatase reaction may be carried out with a reaction mix including 1× CutSmart buffer (50 mM potassium acetate, 20 mM Tris-acetate, 10 mM magnesium acetate, 100 μg/mL BSA), 0.1 U/μL Quick calf intestinal alkaline phosphatase (CIP) for 5′ at 37° C. followed by heat inactivation at 80° C. for 2′.

The M3 construct provide the template for Xpandomer synthesis, which may be carried-out as described herein and with reference to FIG. 11C. The extension oligonucleotides may, in some embodiments, provide additional features for selective release from the support and nanopore translocation, as described throughout the present disclosure.

On-Card Two-Zoned Mirrored Library Production

In this embodiment, a microfluidic chip, i.e. card, is designed with two physically discrete zones for mirrored library workflow, including a first zone for capture of the library fragment and production of the M3 template construct, and a second zone for Xpandomer synthesis. Separating the workflow into two zones in this manner offers several advantages, e.g., obviating the need for bystander extension oligonucleotides.

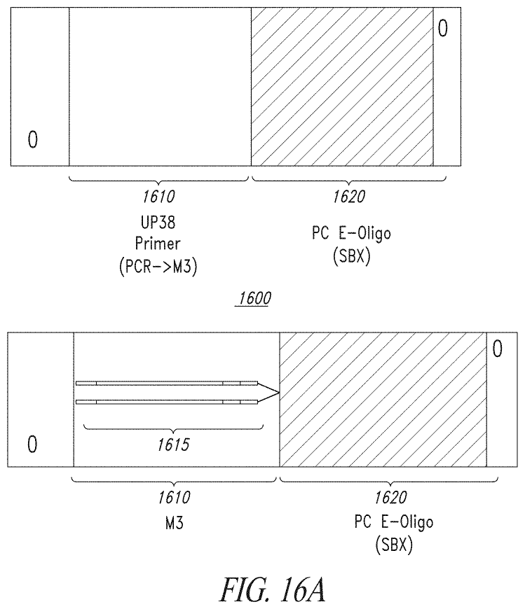

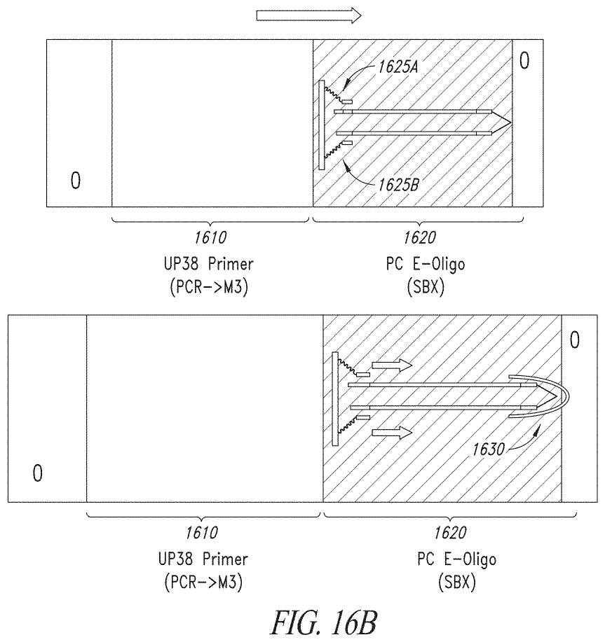

One embodiment of a two-zone card configuration is depicted in FIG. 16A. Here, card 1600 is divided into physically discrete compartments, 1610 and 1620, termed “zone 1” and “zone 2”, respectively. Zone 1 1610 is dedicated to the production of the M3 template construct, while zone 2 1620 is dedicated to Xpandomer synthesis. A capture oligonucleotide structure, such as the UP38 primer described herein, is immobilized on the surface of zone 1, e.g., through click chemistry. An extension oligonucleotide for Xpandomer synthesis is immobilized on the surface of zone 2 in the same manner. In some embodiments, the extension oligonucleotide may include a photocleavable, acid cleavable, or enzymatically cleavable element for selective release of Xpandomer products. Production of the M3 template construct is carried out in zone 1 as described herein. Briefly, the tagged library fragment and PCR mix are added to zone 1 and on-chip PCR is performed to join the tagged library fragment to the capture oligonucleotide; the M1 precursor is formed by A-tailing the library fragment followed by ligation of the trident adaptor; the Trident adaptor is extended by a DNA polymerase to produce the M2 precursor; and the M2 precursor construct is subjected to uracil cleavage followed by exonuclease digestion to cleave the capture oligonucleotide and remove the middle strand, thus generating the M3 template construct 1615.

FIG. 16B illustrates the transfer of the M3 template precursor from zone 1 to zone 2 of the card whereupon it specifically hybridizes to extension oligonucleotides 1625A and 1625B. Cap adaptor structure 1630 is specifically hybridized to the M3 template construct and Xpandomer synthesis is initiated from the extension oligonucleotides in the direction indicated by the arrows. Details of the structure of the cap adaptor and reaction conditions for Xpandomer synthesis are described throughout the present disclosure.

In an alternative embodiment, the capture oligonucleotide bound in zone 1 is designed to include a photocleavable element in place of the uracil residues. In this embodiment, treatment of the M2 precursor with UV light cleaves the capture oligonucleotide and provides the 5′ substrate for exonuclease digestion to produce the M3 template construct. During photocleavage, the zone 2 compartment may be protected from exposure with a UV-blocking interface. An exemplary capture oligonucleotide including a photocleavable element may have the following structure: [azide]D_{10_}L_{30_}Z_{6_}PC_UP038, in which the polymers of D, L, and Z moieties, e.g., “spacers” form a flexible linker, “PC” represents the photocleavable element, and UP038 represents an oligonucleotide with the sequence 5′ TCATAAGACGAACGGAGACT 3′ (SEQ ID NO:13), which is designed to hybridize with the tag sequence of the library fragment.

Bead-Based Mirrored Library Production

This embodiment describes a workflow in which the M3 template construct is produced by a series of steps that are carried out on a bead-based support. In this embodiment, the various constructs are attached to the beads by streptavidin-biotin linkages, as discussed with reference to FIG. 4A. Beads offer certain advantages as a solid substrate, e.g., they are amenable to PCR conditions and are highly scalable, therefore providing increased product yield over other substrates.

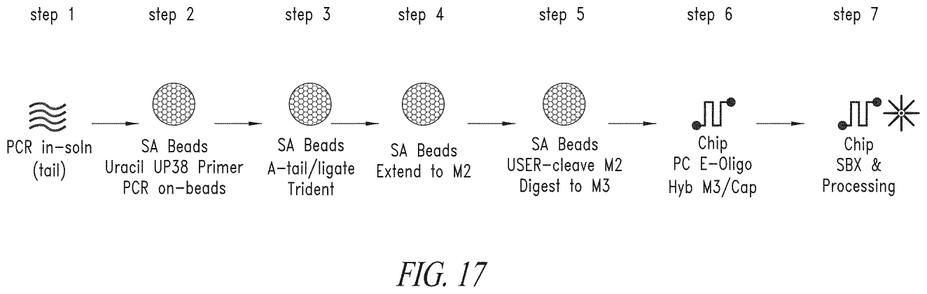

One embodiment of a bead-based work-flow is summarized in FIG. 17. Advantageously, the beads can be washed between steps to remove excess reagents. In step 1, the library fragments are tagged via in-solution PCR, as described herein and with reference to FIG. 14A. In step 2, on-bead PCR is performed to produce the tagged library fragment on the capture oligonucleotide. In this embodiment, the capture oligonucleotide includes a biotin moiety for attachment to the SA-beads. Any suitable SA bead substrate may be used, e.g., Dynabeads® MyOneC1 SA, available from ThermoFisher Scientific. A 35 cycle PCR reaction using KAPA HiFi Uracil+ polymerase will produce up to 1-20 pmol of the bead-bound amplicon from an input of up to 10⁶ copies. Following step 2, the beads are treated with proteinase K for 5′ at 55° C. then washed with a post-PCR wash (1M NaCl, 10 mM Tris, 0.1% Tween-20). In another embodiment, in-solution PCR may be performed using the biotinylated capture oligonucleotide, followed by a spin column-based PCR purification. The purified biotinylated amplicon can then be bound t0SA beads. In step 3, a 3′ A “tail” is added to the library fragments followed by ligation of the Trident adaptor, which includes a 5′ T overhang. An exemplary A-tailing reaction includes 2.5 U MolTaq enzyme and 1 mM dATP and is incubated at 65° C. for 30′. An exemplary ligation reaction includes the Trident adaptor construct (with “T” overhangs), 30 U/μl T4 DNA ligase, 2 U/μl T4 PNK, and 3 U/μl 5′ deadenylase and is incubated at room temperature for 20′. In step 4, the Trident adaptor is extended to generate the M2 precursor. An exemplary extension reaction includes KAPA HiFi U+ polymerase in 1× ReadyMix that is commercially available from Roche. Following step 4, the beads are again treated with proteinase K and washed. In step 5, the M3 template construct is generated by nicking the uracil moiety in the M2 precursor to produce a free 5′ end in the middle strand of the construct followed by exonuclease digestion of this strand. An exemplary nicking/digestion reaction includes 0.1 U/μl USER® and 0.3 U/μl Lambda exonuclease and is incubated for 15′ at 37° C. The exonuclease can then be inactivated by incubating the beads at 75° C. for 10′. In step 6, the free M3 template precursor and the cap adaptor construct are added to a microfluidic chip that includes covalently bound extension oligonucleotide. The M3 construct specifically hybridizes to the extension oligonucleotide and the cap adaptor. In step 7, Xpandomer synthesis and processing reactions are carried out, as described throughout the present disclosure. The final Xpandomer products can be released from the chip by photocleavage. In an alternative embodiment, steps 6 and 7 can also be carried-out on a bead-based support.

Solid-State Xpandomer Synthesis with Branched Extension Oligonucleotides

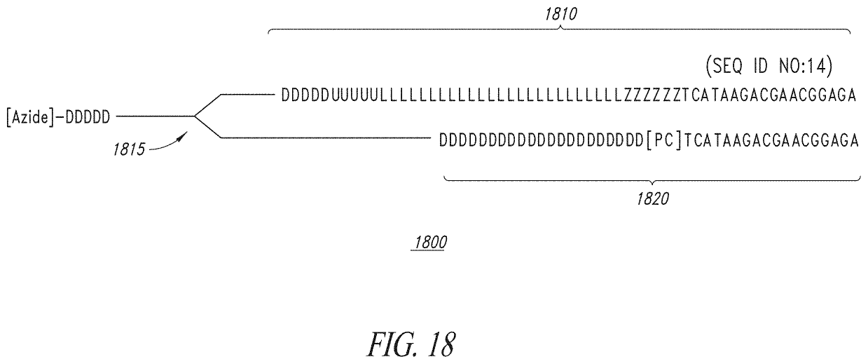

As discussed herein, the sequencing by expansion (SBX) protocol developed by the inventors utilizes extension oligonucleotides (EOs) for Xpandomer synthesis that include several features that perform unique functions during Xpandomer synthesis, processing, and nanopore translocation. For example, in certain embodiments, the 5′ end of the EO provides a “leader” sequence that initiates threading of the final Xpandomer product through a nanopore. Leader sequences may include polymers of C2 (represented herein as “L”), e.g., L₂₅. In some circumstances, it would be desirable to produce a population of mirrored Xpandomers in which only full-length copies thread through the nanopore and generate sequence information. To achieve this goal, the inventors have designed a branched extension oligonucleotide that includes a first and a second extension oligonucleotide joined by a chemical brancher. In this embodiment, only one of the EOs includes a leader sequence and each EO includes a unique selectively cleavable element. One embodiment of a branched EO is illustrated in FIG. 18.

FIG. 18 depicts branched EO 1800 that includes first EO 1810 and second EO 1820 joined by brancher 1815. Branched EO 1800 may be synthesized by conventional phosphoramidite chemistry using an asymmetrical chemical brancher. In this embodiment, only first EO 1810 includes a leader sequence, represented by the polymer of “L” units (wherein “L” symbolizes C2 spacers). Likewise, only the first EO includes a polymer of “Z” units (wherein “Z” symbolizes C12 spacers). The polymer of Z units also plays a role in nanopore translocation. In this embodiment, the first EO includes a polymer of uracil (“U”) residues, which enables selective cleavage of the EO via, e.g., USER®, and the second EO includes a photocleavable element (“PC-spacer”) for UV-mediated cleavage. The sequences of the 3′ oligonucleotide primers (SEQ ID NO:14) of each EO are the same and are designed to hybridize with the M3 template construct. In some embodiments, the oligonucleotide primers are synthesized using one or more 2′-OMe base analogs. The inventors have found that, advantageously, variants of DP04 polymerase used in Xpandomer synthesis are able to utilize 2′-OMe analogs as substrates. The branched EO includes a 5′ terminal azide group for click attachment to a substrate. The length of the L, Z, D, and U polymers depicted in this exemplary embodiment are not intended to be limiting; the present invention is understood to contemplate a variety of suitable polymer lengths and branched EO structures.

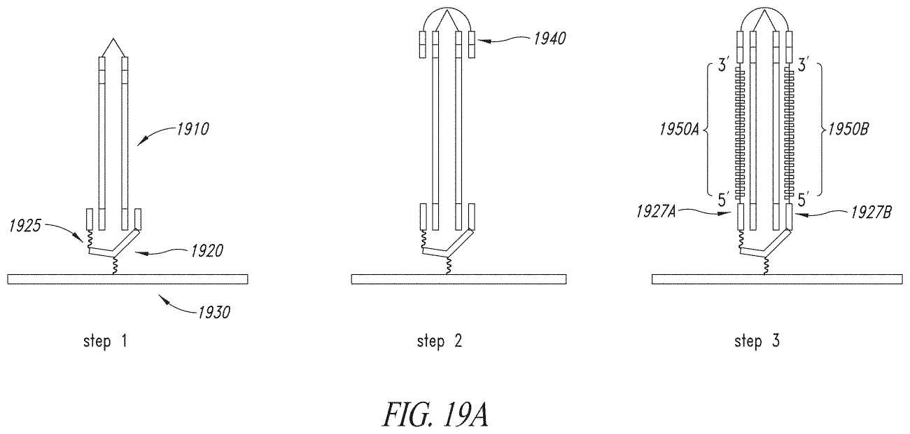

FIGS. 19A and 19B illustrate how the branched EO enables production and isolation of a population of full-length Xpandomers for nanopore sequencing. In step 1, M3 template construct 1910 is hybridized to branched EO 1920 bound to support 1930. Only one EO of the branched structure includes leader sequence 1925. In step 2, cap adaptor structure 1940 is hybridized to the M3 template construct. In step 3, Xpandomer copies 1950A and 1950B are synthesized by extension off oligonucleotide primers 1927A and 1927B. The 3′ ends of the Xpandomers are joined to the free ends of the cap primer construct through end-capping, as described herein. In step 4, the Xpandomer is subjected to USER® treatment, which selectively cleaves the first extension oligonucleotide, exposing the leader sequence 1925. In step 5, the Xpandomer is cleaved and processed to transition from the “constrained” to the “expanded” configuration. In this step, incomplete or truncated Xpandomer by-products can be washed away. In step 6, the Xpandomer is released from the substrate by photocleavage of the second extension oligonucleotide. Advantageously, only full-length Xpandomers 1950 include leader sequence 1925 and will thread through a nanopore and provide sequence information.

All references disclosed herein, including patent references and non-patent references, are hereby incorporated by reference in their entirety as if each was incorporated individually.

It is to be understood that the terminology used herein is for the purpose of describing specific embodiments only and is not intended to be limiting. It is further to be understood that unless specifically defined herein, the terminology used herein is to be given its traditional meaning as known in the relevant art.

Reference throughout this specification to “one embodiment” or “an embodiment” and variations thereof means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents, i.e., one or more, unless the content and context clearly dictates otherwise. It should also be noted that the conjunctive terms, “and” and “or” are generally employed in the broadest sense to include “and/or” unless the content and context clearly dictates inclusivity or exclusivity as the case may be. Thus, the use of the alternative (e.g., “or”) should be understood to mean either one, both, or any combination thereof of the alternatives. In addition, the composition of “and” and “or” when recited herein as “and/or” is intended to encompass an embodiment that includes all of the associated items or ideas and one or more other alternative embodiments that include fewer than all of the associated items or ideas.

Unless the context requires otherwise, throughout the specification and claims that follow, the word “comprise” and synonyms and variants thereof such as “have” and “include”, as well as variations thereof such as “comprises” and “comprising” are to be construed in an open, inclusive sense, e.g., “including, but not limited to.” The term “consisting essentially of” limits the scope of a claim to the specified materials or steps, or to those that do not materially affect the basic and novel characteristics of the claimed invention.

The abbreviation, “e.g.” is derived from the Latin exempli gratia, and is used herein to indicate a non-limiting example. Thus, the abbreviation “e.g.” is synonymous with the term “for example.” It is also to be understood that as used herein and in the appended claims, the singular forms “a,” “an,” and “the” include plural reference unless the context clearly dictates otherwise, the term “X and/or Y” means “X” or “Y” or both “X” and “Y”, and the letter “s” following a noun designates both the plural and singular forms of that noun. In addition, where features or aspects of the invention are described in terms of Markush groups, it is intended, and those skilled in the art will recognize, that the invention embraces and is also thereby described in terms of any individual member and any subgroup of members of the Markush group, and Applicants reserve the right to revise the application or claims to refer specifically to any individual member or any subgroup of members of the Markush group.

Any headings used within this document are only being utilized to expedite its review by the reader, and should not be construed as limiting the invention or claims in any manner. Thus, the headings and Abstract of the Disclosure provided herein are for convenience only and do not interpret the scope or meaning of the embodiments.

Where a range of values is provided herein, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

For example, any concentration range, percentage range, ratio range, or integer range provided herein is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means±20% of the indicated range, value, or structure, unless otherwise indicated.

All of the U.S. patents, U.S. patent application publications, U.S. patent applications, foreign patents, foreign patent applications and non-patent publications referred to in this specification and/or listed in the Application Data Sheet, are incorporated herein by reference, in their entirety. Such documents may be incorporated by reference for the purpose of describing and disclosing, for example, materials and methodologies described in the publications, which might be used in connection with the presently described invention. The publications discussed above and throughout the text are provided solely for their disclosure prior to the filing date of the present application. Nothing herein is to be construed as an admission that the inventors are not entitled to antedate any referenced publication by virtue of prior invention.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

Furthermore, the written description portion of this patent includes all claims. Furthermore, all claims, including all original claims as well as all claims from any and all priority documents, are hereby incorporated by reference in their entirety into the written description portion of the specification, and Applicants reserve the right to physically incorporate into the written description or any other portion of the application, any and all such claims. Thus, for example, under no circumstances may the patent be interpreted as allegedly not providing a written description for a claim on the assertion that the precise wording of the claim is not set forth in haec verba in written description portion of the patent.

The claims will be interpreted according to law. However, and notwithstanding the alleged or perceived ease or difficulty of interpreting any claim or portion thereof, under no circumstances may any adjustment or amendment of a claim or any portion thereof during prosecution of the application or applications leading to this patent be interpreted as having forfeited any right to any and all equivalents thereof that do not form a part of the prior art.

Other nonlimiting embodiments are within the following claims. The patent may not be interpreted to be limited to the specific examples or nonlimiting embodiments or methods specifically and/or expressly disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

### EXAMPLES

### Example 1

### Solid-State Xpandomer Synthesis—Direct Conjugation of Extension Oligonucleotide to Microfluidic Chip

This example describes solid-state synthesis of Xpandomers, which are expandable copies of a single-stranded polynucleotide template comprised of XNTP nucleotide analogs, and possess unique features for improved nanopore sequencing. Solid-state Xpandomer synthesis was conducted on a microfluidic chip substrate functionalized by covalent linkage of an extension oligonucleotide (the “E-oligo”) to the chip. Polymerase-mediated extension of the bound E-oligo with XNTPs generates Xpandomer products that remain attached to the chip and can be washed, processed, and released in an efficient and controlled manner.

The E-oligo utilized in this experiment (“E52 SIMA PC azide”) included the following features: a 5′ azide group followed by a polymer of PEG-6 monomers, a photocleavable spacer, a “leader” polymeric sequence, a “concentrator” polymeric sequence, a fluorescently labeled nucleotide, and the oligonucleotide primer. The leader and concentrator polymers function, e.g., to improve the efficiency of Xpandomer translocation through a nanopore sensor and are described in more detail in Applicants' issued U.S. Pat. No. 9,670,526, entitled “Concentrating a target molecule for sensing by a nanopore”, which is herein incorporated by reference in its entirety.

A. Chip Functionalization

A commercially available continuous flow PCR chip fabricated from Zeonor (a cyclo-olefin thermoplastic polymer) was used as the solid support in this experiment. Chips were functionalized with an alkyne moiety using the direct conjugation by photoabstraction protocol described herein. Briefly, chips were primed with 350 μL of 80% DMS; then 60 μL of 10 mM propargyl maleimide in 80% DMSO was added and the chips were incubated 20 minutes under a 20W UV lamp; chips were then washed successively with 300 μL of 80% DMSO, 300 μL of 100% DMF, 300 μL water, 300 μL of a solution of 300 mM Na₂HPO₄, 1% Tween-20, and 0.5% SDS, and incubated 5 minutes at 37° C.; chips were finally washed with 300 μL water, followed by 300 μL of 3×PBS.

B. Click Reaction

Solutions for the click reaction were prepared as follows: 1) a catalyst mix was prepared by mixing 5.0 μL water, 1.5 μL 100 mM THPTA, 1.5 μL 100 mM sodium ascorbate, 0.5 μL 10 mM CuSO4, 0.5 μL 100 mM aminoguanidine, and 1.0 μL 100% DMF and incubated for 5-15 minutes at room temperature; 2) a substrate mix was prepared by mixing 29.22 μL water, 4.00 μL 100% DMF, 1.25 μL 1000 mM sodium phosphate, pH 7.0, 0.78 μL 25.6 μM extension oligonucleotide (20 pmol E52 SIMA PC azide), 1.25 μL 100 mM MgCl2, 2.0 μL 100 mM aminoguanidine, and 1.5 μL 100 mM sodium ascorbate; 3) the substrate mix was added to the catalyst mix and vortexed. Functionalized chips were washed with 300 μL water and 50 μL of the click reaction mixture was added, followed by incubation for 20 minutes at room temperature.

C. Extension Reactions

For the extension reaction, a ratio of 20 pmol:20 pmol of DNA template to E-oligo was used. The template was a single-stranded 100mer sequence derived from the HIV2 genome; the sequence of the E-oligo primer was 5′ TCATAAGACGAACGGA 3′ (SEQ ID NO:4). The single-stranded DNA template molecules were hybridized to the support-bound E-oligos by incubating 20 pmol template with the chip for 5 minutes at 37° C., followed by wash with 300 μL MEB buffer.

Extension reactions included the following reagents: 4 nmol XNTPs, 0.08 mM polyphosphate, 0.6 mM MnCl₂, 0.5M betaine, 0.25M urea, 10 μg single-strand binding protein (SSB), 9 μg DNA polymerase protein (C4760) 1.4 mM PEM combo (AZ8-8 and AZ43-43). The final reaction volume was brought to 50 μL with 5% NMS and the extensions were run at 42° C.

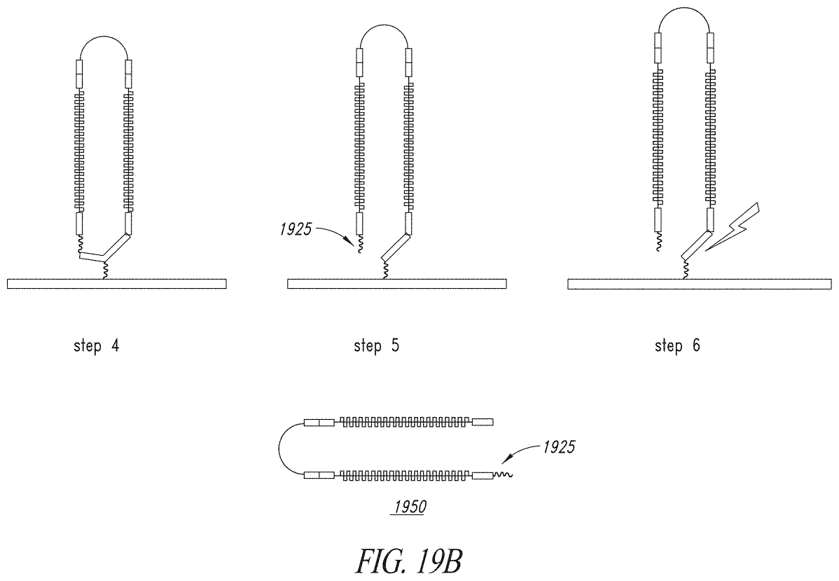

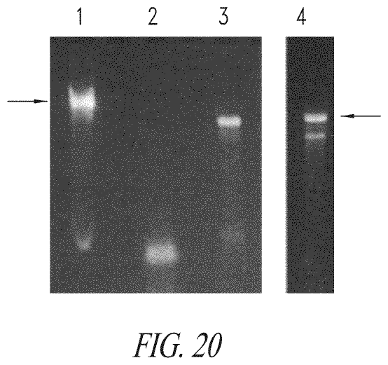

Following extension, the chips were treated and washed to remove the extension reagents, bound Xpandomer products were released from the chip by photocleavage (15 minute treatment with a Firefly UV curing lamp) and cleaved Xpandomer products were eluted from the chip in 60 μL 40% acetonitrile. Xpandomer products were analyzed by gel electrophoresis by running ˜0.75 pmol product per lane in a 2.5% Nusieve gel with 1×TAE buffer. A representative gel is shown in FIG. 20 in which the products of the solid-state Xpandomer synthesis are shown in lane 3 with the full-length product denoted by the arrow. For reference, the products of an Xpandomer synthesis reaction conducted in-solution, using an identical template, are shown in lane 1. The tighter band observed in lane 3 suggests that solid-state Xpandomer synthesis may improve product distribution, with a reduction in partial or truncated products (the apparent larger size of the smeared band in lane 1 reflects a difference in the composition of the E-oligo used in the solution-based extension reaction). Lane 2 is a negative control in which the template used does not hybridize to the E-oligo and lane 4 is a positive control showing products of a solid-state extension carried out under different reaction conditions. These results demonstrate proof-of-concept for sold-state synthesis of Xpandomers.

### Example 2

### Solid-State Xpandomer Synthesis for Sequencing by Expansion (SBX)

This example describes solid-state synthesis and processing of Xpandomer copies of a 222mer template followed by sequencing of the products using a nanopore sensor system. All steps of the workflow prior to sequencing were carried out with Xpandomer intermediates and final products bound to the substrate. This protocol provides numerous advantages over a solution-based workflow, e.g., the ability to sequentially add pure reagents for each of the reactions in reduced volumes. In this experiment, the Xpandomer extension reaction was performed on a microfluidic chip substrate primed by direct covalent linkage of the E-oligo. Functionalization of the chip and click attachment of the E-oligo were conducted as described in Example 1.

A. Extension Reaction

The extension reaction was conducted with a molar ratio of 10 pmol:20 pmol of DNA template to E-oligo. The template used was a single-stranded 222mer sequence derived from the HIV2 genome and the E-oligo used was the E52 oligo described in Example 1. The single-stranded DNA template molecules were hybridized to the bound E-oligo by incubating 10 pmol template with the chip in a solution of 500 mM NH₄OAc, 5% NMS, 1M urea, and 2% PEG 8K for 5 minutes at 37° C., followed by wash with 300 μL MEB buffer. Prior to the extension reaction, the chip was washed with 300 μL of a solution of 50 mM TrisCl, 200 mM NH₄OAc, 5% NMS, 10% PEG 8K, and 1M urea.

Extension reactions included the following reagents: 4 nmol XNTPs, 0.08 mM polyphosphate, 0.6 mM MnCl₂, 0.5M betaine, 0.25M urea, 10 μg single-strand binding protein (SSB), 9 μg DNA polymerase protein (C4760), 1.0 mM AZ-8,8 and 4 mM AZ-43,43 PEM additives. The final reaction volume was brought to 50 μL with 5% NMS and a buffer composed of 50 Mm Tris HCl, pH 8.84, 200 mM NH₄OAc, pH 6.73, and 20% PEG. The extension reactions were run for 30 minutes at 42° C.

Following extension, the chip was washed three times with 300 μL of a wash solution containing 100 mM HEPES, pH8.0, 100 mM Na₂HPO4, 1% Tween 20, 3% SDS, 15% DMF, and 5 mM EDTA in D₂O.

B. Xpandomer Processing

Bound extension products were first treated with acid to break the phosphoramidite bonds in the Xpandomers in order to linearize the molecules, as illustrated, e.g., in FIG. 1C. Acid-mediated cleavage was accomplished by adding 2004 of a solution of 7.5M DCI in D₂O to the chip and incubating for 30 minutes at room temperature. The bound products were then neutralized and washed by adding 9004 of a solution of 100 mM HEPES, pH 8.0, 100 mM Na₂HPO₄, pH 8.0, 1% Tween-20, 3% SDS, 15% DMF, and 5 mM EDTA in D₂O. The bound products were then modified by adding 300 μL of a solution of 100 mM HEPES, pH 8.0, 100 mM Na₂HPO₄, pH 8.0, 1% Tween 20, 3% SDS, 15% DMF, 5 mM EDTA in D₂O while 200 μmol succinate anhydride (loaded separately in a syringe) was added directly to the chip, followed by incubation at 23° C. for five minutes. The modified products were then washed with 500 μL of a solution of 15% ACN and 5% DMSO in H₂O.

C. Release of Xpandomers from the Chip

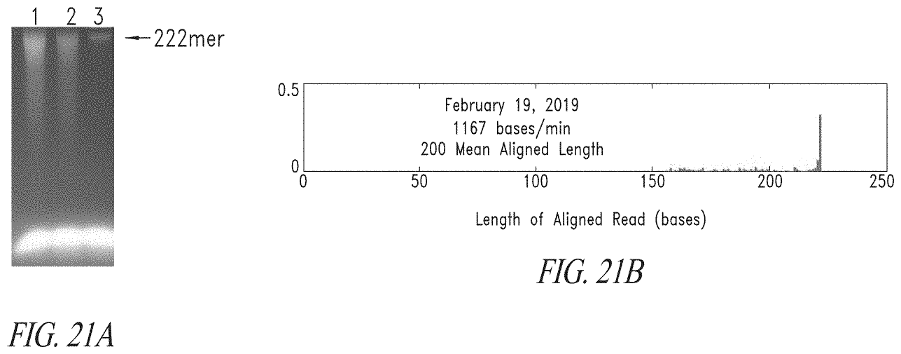

Bound Xpandomer products were released from the chip substrate by photocleavage. First, 60 μL of a solution of 15% ACN and 5% DMSO in H₂O was added to the chip, then the chip was subjected to irradiation for 15 minutes using a UV curing lamp. Released Xpandomers were eluted from the chip with a solution of 5% DMS and 15% acetonitrile. The eluted material was first analyzed by gel electrophoresis as shown in FIG. 21A. 15% of the sample was run in lane 3 of the gel (2.5% NuSieve agarose in 0.5× TBE) with the full-length Xpandomer product denoted by the arrow. For reference, products of solution-based Xpandomer synthesis reactions using the same template are shown in lanes 1 and 2. As can be seen, solid-phase synthesis produces a tighter band compared to solution-based synthesis, indicating a larger percentage of full-length product in the sample.

D. Nanopore Sequencing

For sequencing, protein nanopores are prepared by inserting α-hemolysin into a DPhPE/hexadecane bilayer member in buffer B1, containing 2M NH₄Cl and 100 mM HEPES, pH 7.4. The cis well is perfused with buffer B2, containing 0.4M NH₄Cl, 06 M GuCl, and 100 mM HEPES, pH 7.4. The Xpandomer sample is heated to 70° C. for 2 minutes, cooled completely, then a 2 μL sample is added to the cis well. A voltage pulse of 90 mV/390 mV/10 μs is then applied and data is acquired via Labview acquisition software.

Sequence data is analyzed by histogram display of the population of sequence reads from a single SBX reaction. The analysis software aligns each sequence read to the sequence of the template and trims the extent of the sequence at the end of the reads that does not align with the correct template sequence. A representative histogram of nanopore sequencing of the 222mer template is presented in FIG. 21B. Notably, solid-state synthesis and processing produced Xpandomer products generating highly accurate sequence reads across the entire length of the 222mer molecules when read by a nanopore sensor.

### Example 3

### Xpandomer Synthesis with End-Capping

This example describes end-capping of Xpandomers products during synthesis and efforts to optimize the process with different reaction additives. The template used in the following experiment was a 121mer sequence derived from the HIV2 genome and the E-oligo (“EO”) used was the E52 RD with the following features: a 5′ SIMA (fluorescent tag) following by a leader polymer, a concentrator polymer, and an oligonucleotide primer with the sequence, 5′ TCATAAGACGAACGGA 3′ (SEQ ID NO:4). The end cap includes a terminal oligonucleotide with the following sequence, 5′ K[GCGTTAGGTCCCAGTGTTTAC(SEQ ID NO:15)]X 3′, where K represents a G clamp and X represents a PEG3 moiety. The terminal oligonucleotide is complementary to, and hybridizes with, the 5′ end of the template. The 5′ end of the terminal oligonucleotide is linked to a ddCTP cap via the linker illustrated in feature 710A of FIG. 7A to form the complete end cap structure.

In this experiment, five extension reactions were run, each of which included the following reagents: a 1:1 molar ratio of template to E-oligo, 2 mM AZ-8,8 and 10 mM AZ-43,43 PEM additives, 5% NMS, 1.8 μg DNA polymerase, 0.08 mM XNTPs, 0.08 mM polyphosphate, and 0.6 mM MnCl₂. Reactions 2-5 included a two-fold molar excess of end cap relative to the template and EO, while reaction 1 did not include the end cap. The reactions also included various additives, as follows. Reaction 1: 0.5M betaine, 0.25M urea, and 2 μg single-strand binding protein (SSB); reaction 2: 0.5M betaine, 0.25M urea, and 2 μg SSB; reaction 3: 0.25M urea; reaction 4: 0.5M betaine and 0.25M urea; reaction 5: 0.25M urea and 2 μg SSB. The final reaction volume of each was 10 μL and reactions were run at 42° C.

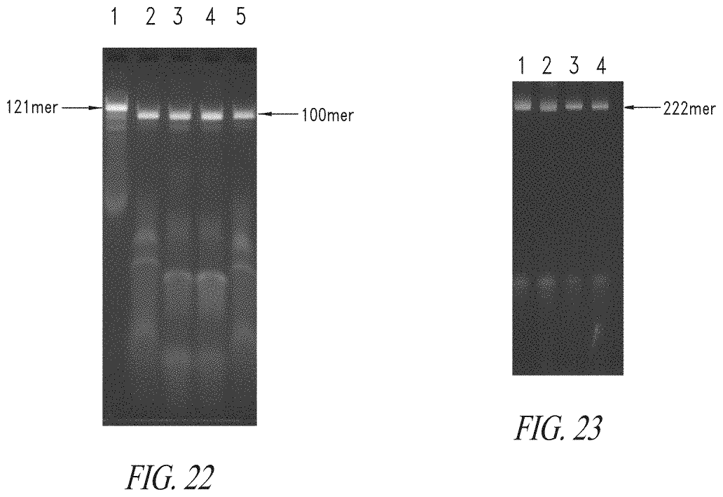

Products of the extension reaction were analyzed by gel electrophoresis, as shown in FIG. 22. Lane 1 shows the product of reaction 1, which did not include the end cap. In this reaction, the SIMA dye is linked to the EO and the extension product is a 121mer Xpandomer. Lanes 2-5 show the products of reactions 2-5, respectively, which each included the end cap. In these reactions, the SIMA dye is linked to the end cap, in contrast to reaction 1. As can be seen, in each of reactions 2-5, the end cap has been successfully joined to the Xpandomer by the DNA polymerase, indicating that the Xpandomer represents a complete copy of the DNA template. Due to incorporation of the terminal oligonucleotide of the end cap into the extension product, the products of reactions 2-5 are 100mer Xpandomers and migrate more quickly on the gel than the 121mer of reaction 1. These results show remarkably tight Xpandomer bands on the gel, indicating that the end-capping reaction is very efficient under the experimental conditions tested. Importantly, end-capping provides a means to tag and capture full-length Xpandomers for, e.g., nanopore sequencing.

### Example 4

### Solid-State Xpandomer Synthesis with End-Capping

This example describes solid-state synthesis of a 222mer Xpandomer coupled with end-capping of the full-length product. Solid-state synthesis was conducted on a microfluidic chip substrate functionalized by covalent linkage of an extension oligonucleotide (the “E-oligo”) to the substrate, as described in Example 1. Upon completing a full-length copy of the template, the DNA polymerase encounters the end cap hybridized to the 5′ end of the template and join the 5′ end of the end cap to the 3′ end of the Xpandomer. A fluorescent dye attached to the end cap enables visualization of full-length copies of the template by gel electrophoresis.

A. Extension and end-capping reactions.

The template used in the following experiment was a 243mer sequence derived from the Streptococcus pneumoniae genome and the E-oligo (“EO”) used was an E52 EO including a photocleavable linker and an oligonucleotide primer with the sequence, 5′ TCATAAGACGAACGGA 3′ (SEQ ID NO:4). The end cap includes a terminal oligonucleotide with the following sequence, 5′ K[GCGTTAGGTCCCAGTGTTTAC(SEQ ID NO:15)] 3′, where K represents a G clamp. The terminal oligonucleotide is complementary to, and hybridizes with, the 5′ end of the template. The 5′ end of the terminal oligonucleotide is linked to a ddCTP cap via the linker illustrated in feature 710A of FIG. 7A to form the complete end cap structure.

In this experiment, four on-chip extension reactions were run with the same template, primer, and end cap. Reaction 1 included the following reagents: a template:EO:end cap molar ratio of 16:20:32, 0.08 mM XNTPs, 1 mM AZ-8,8 and 4 mM AZ-43,43 PEMs, 9 μg DNA polymerase (DPO4 variant C4760), 10 μg SSB, 0.6 mM MnCl₂, 0.08 mM polyphosphate, 50 mM Tris HCl, pH 8.84, 200 mM NH₄OAc, pH 6.73, 20% PEG, 5% NMS, 0.25M urea, 0.5M betaine. The 50 μL reaction was run 42° C. Reaction 2 included the following reagents: a template:EO:end cap molar ratio of 6:10:12, 0.08 mM XNTPs, 1 mM AZ-8,8 and 4 mM AZ-43,43 PEMs, 9 μg DNA polymerase (C4760), 10 μg SSB, 0.6 mM MnCl₂, 0.08 mM polyphosphate, 50 mM Tris HCl, pH 8.84, 200 mM NH₄OAc, pH 6.73, 20% PEG, 5% NMS, 0.25M urea, 0.5M betaine. The 20 μL reaction was run 37° C. Reaction 3 included the following reagents: a template:EO:end cap molar ratio of 6:10:12, 0.08 mM XNTPs, 1 mM AZ-8,8 and 4 mM AZ-43,43 PEMs, 9 μg DNA polymerase (C4760), 10 μg SSB, 0.6 mM MnCl₂, 0.08 mM polyphosphate, 50 mM Tris HCl, pH 8.84, 200 mM NH₄OAc, pH 6.73, 20% PEG, 5% NMs, 0.25M urea, 0.5M betaine. The 25 μL reaction was run 42° C. Reaction 4 included the following reagents: a template:EO:end cap molar ratio of 10:10:20, 0.08 mM XNTPs, 1 mM AZ-8,8 and 4 mM AZ-43,43 PEMs, 9 μg DNA polymerase (C4760), 10 μg SSB, 0.6 mM MnCl₂, 0.08 mM polyphosphate, 50 mM Tris HCl, pH 8.84, 200 mM NH₄OAc, pH 6.73, 20% PEG, 5% NMS, 0.25M urea, 0.5M betaine. The 25 μL reaction was run 42° C.

Products of the extension reaction were analyzed by gel electrophoresis on a 2.5% NuSieve agarose gel, as shown in FIG. 23. Lanes 1-4 show the products of reactions 1-4, respectively, which each included the end cap. In these reactions, the SIMA dye is linked to the end cap. As can be seen, in each reaction the end cap has been successfully joined to the Xpandomer by the DNA polymerase, indicating that the Xpandomer represents a complete copy of the DNA template. These results show remarkably tight Xpandomer bands on the gel, indicating that the end-capping reaction is also very efficient during solid-state synthesis. Interestingly, the efficiency of extension and capping appears to be influenced by the nature of the additives present in the reaction. These results indicate that solid-state synthesis of Xpandomers can be optimized through trial and error.

### Example 5

### Mirrored Library Construction—Ligation of Trident Adaptor to Library Insert

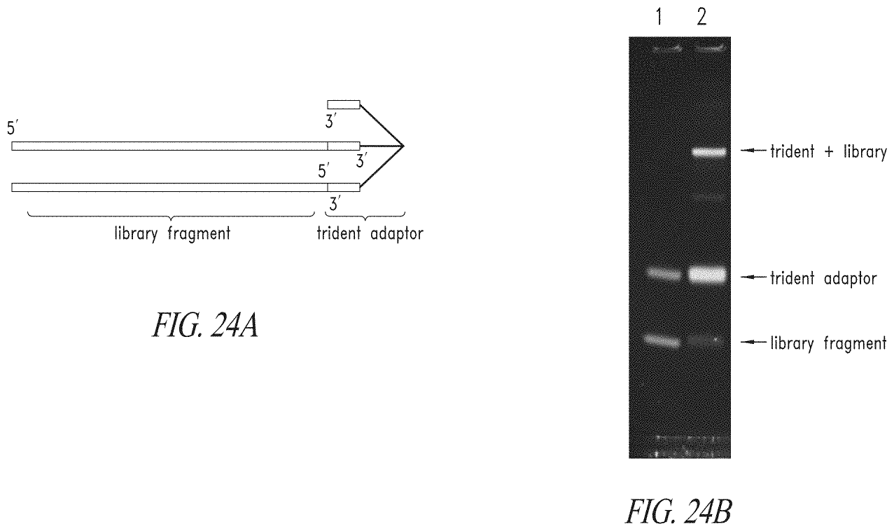

This Example describes an initial step in generating the mirrored library constructs of the present invention, in which the trident adaptor is ligated to a library fragment of double-stranded DNA. FIG. 24A illustrates the basic structural features of the constructs used in this experiment. The library fragment is a double-stranded 60mer sequence derived from the HIV2 genome, in which the “minus” strand (corresponding to the top strand in the illustration) and the “plus” strand (corresponding to the bottom strand in the illustration) incorporate a 3′ single base overhang. The polarity of the library strands is denoted by “5′” numbering in the illustration. The trident adaptor is composed of three DNA strands, as illustrated in FIG. 24A, with the polarity of each strand denoted by “3′” numbering. The top and bottom strands of the trident are 24mer oligonucleotides have identical sequences, while the sequence of the oligonucleotide comprising the middle strand is the reverse complement of top and bottom strand sequences. The top and bottom strands also have 3′ single base overhangs that enable directional ligation to the library fragment. The 5′ ends of the three strands are joined together by a chemical brancher to form the trident adaptor, in which the middle and bottom strands form a double-stranded hybrid, while the top strand remains single-stranded.

In this experiment, the ligation reaction was carried out in-solution with a 5:1 molar ratio of trident adaptor to library fragment. The 15 μL final reaction volume included the following reagents: ligase reaction buffer, 3 mM ATP, 6% glycerol, 6% 1,2-propanediol, 0.1 μM library fragment, 0.5 μM trident adaptor, 1 U/μL PNK, and 120 U/μL DNA ligase. The reaction was run at 15° C. for 5 minutes and ligation products were analyzed by gel electrophoresis in a 6% TBE-U gel stained with SYBR to visualize products. A representative gel is shown in FIG. 24B in which the unligated trident and library reference fragments were run in lane 1 and the products of the ligation reaction were run in lane 2. As can be seen, the ligated trident/library fragment product is clearly distinguishable from the unligated products. Of note, the band corresponding to the unligated library fragment is very faint in lane 2, indicating that the majority of the library fragment has been converted into the trident/library ligate.

### Example 6

### Mirrored Library Construction—Extension from Trident Adaptor and Exonuclease Digestion to Produce Mirrored Library Construct

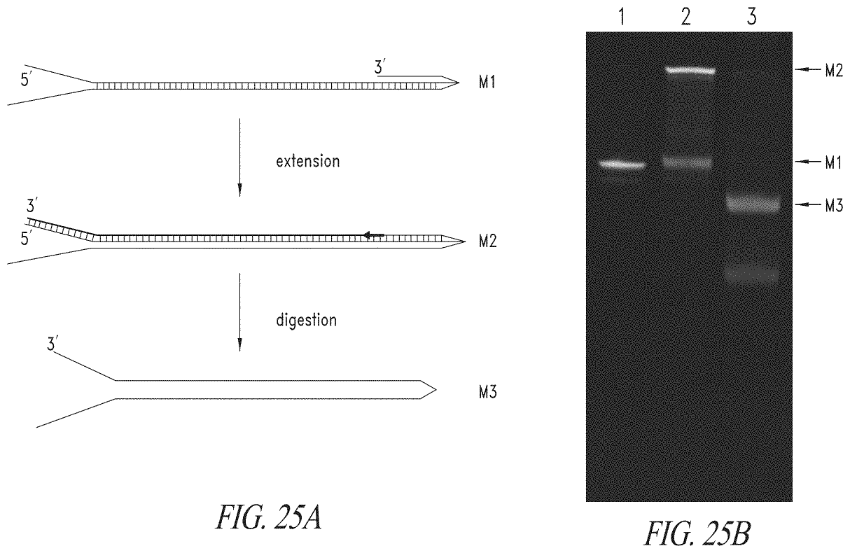

This Example describes the extension and digestion steps in generating the mirrored library constructs, which are depicted in simplified form in FIG. 25A. For the extension step, the single-stranded top strand of the trident adaptor of the M1 construct is used as an extension primer by DNA polymerase to synthesize a new strand of DNA using the library fragment as a template. Extension of the M1 construct produces the M2 construct in the illustration. For the digestion step, the original template strand of M2 (indicated by the 5′ notation) is then removed by exonuclease treatment to produce the M3 construct. M3 includes two identical single-stranded copies of the library fragment “plus” strand and is referred to as a “Mirrored Library Construct”.

Extension reactions were conducted with the following reagents: 0.3 pmol M1 ligation product, 0.2 mM dNTPS, and 0.4 U/μL DNA polymerase (Vent®(exo-)), in Thermo Pol reaction buffer. Vent®(exo-) was chosen as the DNA polymerase for the extension reaction based on an absence of exonuclease activity as well as strong strand-displacing activity. Extension reactions (5 μL total volume) were subjected to an initial denaturation step at 95° C. for two minutes, followed by 25 cycles at 95° C. for 15 seconds and 72° C. for 6 seconds. After the denaturation/extension cycles, the reactions were quenched, denatured, and run on a gel to visualize extension products.

For the digestion reaction, 0.3 pmol M2 extension product was treated with Lambda exonuclease (1 U/μL) in lambda exonuclease reaction buffer. Digestion reactions (10 μL total volume) were run for 5 minutes following exo addition. Digestion products were analyzed by gel electrophoresis as described above. Results of a representative experiment are shown in FIG. 25B. Lane 1 of the gel shows the M1 reference product (0.2 pmol product/lane), while lanes 2 and 3 show products of the extension and digestion reactions, respectively. The large band in lane 2 demonstrates successful conversion of the M1 ligated product to the larger M2 extension product, while the smaller band in lane 3 demonstrates successful conversion of the M2 extension product to the M3 digestion product.

### Example 7

### Solid-State Synthesis of the M1 Mirrored Library Construct

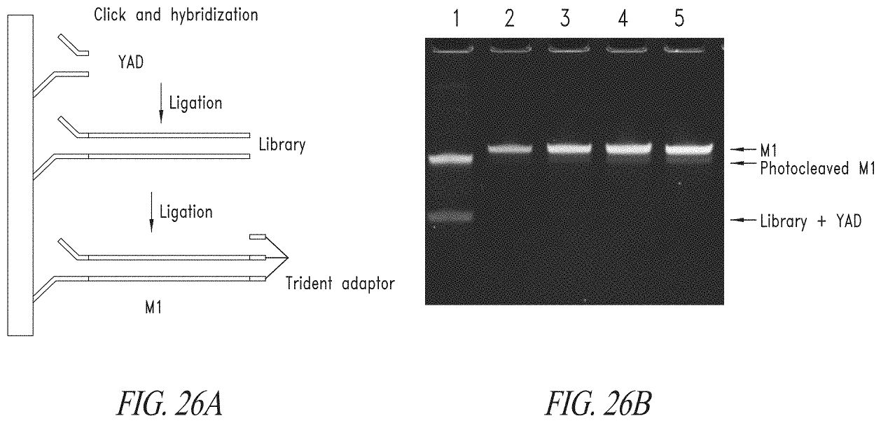

This Example describes the work-flow for building the M1 construct on a solid support. The workflow is illustrated in simplified form in FIG. 26A. In the following experiment, a Y adaptor (“YAD”) was first covalently bound to the support via click chemistry; the library fragment and trident adaptor were then ligated to the bound YAD to produce the M1 construct on the support. M1 was finally released from the support by cleavage of the photosensitive linkage between the YAD and the support.

A. Click attachment of YAD to solid support.

A commercially available continuous flow PCR chip fabricated from Zeonor (a cyclo-olefin thermoplastic polymer) was used as the solid support in this experiment. Chips were functionalized as described in Example 1. A copper click reaction was performed as follows: a 60 μL catalyst mix was prepared by mixing 3 mM THPTA, 6 mM sodium ascorbate, 1 mM CuSO₄, 5 mM aminoguanidine, and 10% DMF; a 1204 substrate mix was prepared by mixing 10% DMF, 25 mM sodium phosphate, pH 7.0, 50 mol of the E6 oligonucleotide arm of the YAD (linked to an azide moiety), 2.5 mM MgCl₂, 5 mM aminoguanidine, and 6 mM sodium ascorbate. 30 μL of the catalyst mix was then added to the substrate mix and 75 μL of this click mix was added to the chip, followed by incubation for 30 minutes at room temperature.

B. Extension of the M1 Construct

Following the click reaction, the chip was washed with water and solution “10002” (300 mM sodium phosphate, pH 8.0, 1% Tween-20, 0.5% SDS, and 1 mM EDTA). A 50 μL E52 YAD mix (containing the second oligonucleotide arm of the Y adaptor) was prepared by mixing 25 μL solution “CHB002” (500 mM NH4OAc, 2% PEG 8K, 1M urea, and 5% NMS) and 100 pmol E52 oligonucleotide and applied to the chip. The chip was incubated for 20 minutes at 30° C. to allow the E52 oligonucleotide to hybridize to the E6 oligonucleotide. The chip was then washed three times with 300 μL CHB002.

To ligate the library fragment and the trident adaptor to the substrate-bound YAD, a 50 μL ligation reaction mix was prepared by combining 15 pmol library insert (the HIV2 60mer), 50 pmol trident adaptor, 11 mM ATP, 1 U/μL T4 PNK, and blunt/T4 ligase master mix (available from NEB). The ligation mix was added to the chip followed by incubation for 15 minutes at 16° C. The ligation mix was then removed from the chip and 5 μL of 5′ deadenylase (50,000 U/mL) was added; subsequently the ligation mix was added back to the chip followed by incubation for 15 minutes at 16° C. The chip was then washed twice with 300 μL CHB002 and then with 300 μL water. Then 300 μL of 10002 was added and the chip was incubated for 5 minutes at 37° C. The chip was then washed three times with 300 μL CHB002 and then with 300 μL water. All liquid was then removed from the chip and 75 μL water was added.

To release the bound product from the chip, the photosensitive linkage of the YAD to the chip was cleaved by exposing the chip to UV light for 15 minutes with a Firefly curing lamp. Released product was eluted from the chip and 1% of the recovered material was analyzed by gel electrophoresis. A representative gel is shown in FIG. 26B. The sample in lane 1 represents 1% of the material recovered from the chip by photocleavage, while the samples in lanes 2-5 are control titrations of purified, uncleaved M1 that was synthesized in-solution. As can be seen, the solid-state synthesis protocol successfully produces the completely assembled M1 mirrored library product.

### Example 8

### Sequencing by Expansion of a Mirrored Library Construct

This Example demonstrates proof-of-concept for mirrored library sequencing by expansion (SBX). The starting material in this experiment was the M1 product built around the HIV2 60mer library fragment described in Example 7. The extension conditions to produce the M2 product were as follows: ˜7.5 pmol M1 product, 0.2 mM dNTPs, and 0.16 U/μL Vent polymerase in Thermopol reaction buffer. The 37.3 μL reaction was incubated at 95° C. for 2 minutes then subjected to 25 cycles at 95° C. for 15 seconds and 72° C. for six seconds. The M2 digestion conditions to produce the M3 product were as follows: the 36.68 μL extension reaction was treated with 0.26 U/μL Lambda exonuclease in Lambda exo buffer. The reaction was run for five minutes at 37° C. then heat inactivated to produce the M3 mirrored library construct.

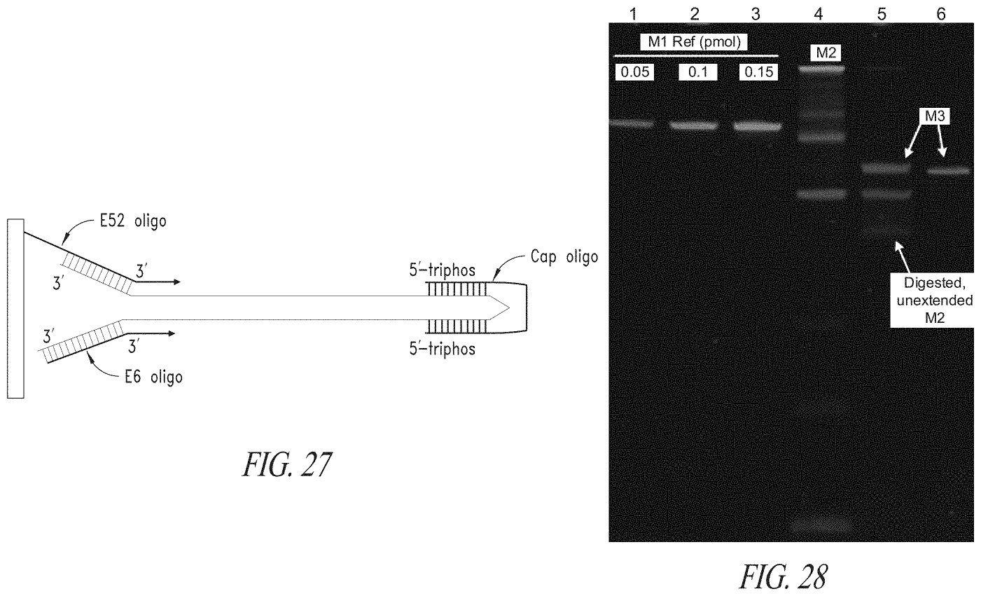

Production of Xpandomer copies of the M3 product was conducted by solid-state synthesis. As an initial step, the M3 digestion product was hybridized to a microfluidic chip, as illustrated in FIG. 27. In this experiment, the chip was primed by click attachment of an E52 oligonucleotide designed to hybridize to the top arm of the M3 YAD. The E52 oligonucleotide provides a primer for the synthesis of a copy of the top strand of the M3 construct, as indicated by the arrow in FIG. 27. To hybridize the M3 digestion product to the chip, and create a template for Xpandomer extension, 42.754 of the digestion reaction was mixed with 10 pmol E6 oligonucleotide (designed to hybridize to the bottom strand arm of the YAD and provide a primer for the synthesis of a copy of the bottom strand of the M3 construct) and 10 pmol cap oligonucleotide (designed to hybridize to the M3 trident adaptor and provide free 5′ triphosphates for end-capping of each copy of the M3 library fragment) in a hybridization buffer composed of 200 mM NH₄OAC, pH 6.62, 2% PEG8K, and 0.25M urea. The 50 μL hybridization reaction was incubated at 95° C. for 15 seconds then added to the chip, which had been warmed to 65° C. The chip was then brought to 37° C. and incubated for five minutes.

A representative gel showing samples from the mirrored library workflow is shown in FIG. 28. Lanes 1-3 of the gel show reference samples of purified M1 product (0.5, 0.1, and 0.15 pmol M1, respectively). Lane 4 represents 1.3% of the extension reaction producing the M2 product and lane 5 represents 1.2% of the digestion reaction producing the M3 product. Lane 6 represents 5% of the M3 material retained on the chip after hybridization. Importantly, only the complete M3 product was retained on the chip, despite the presence of secondary products in the digestion reaction.

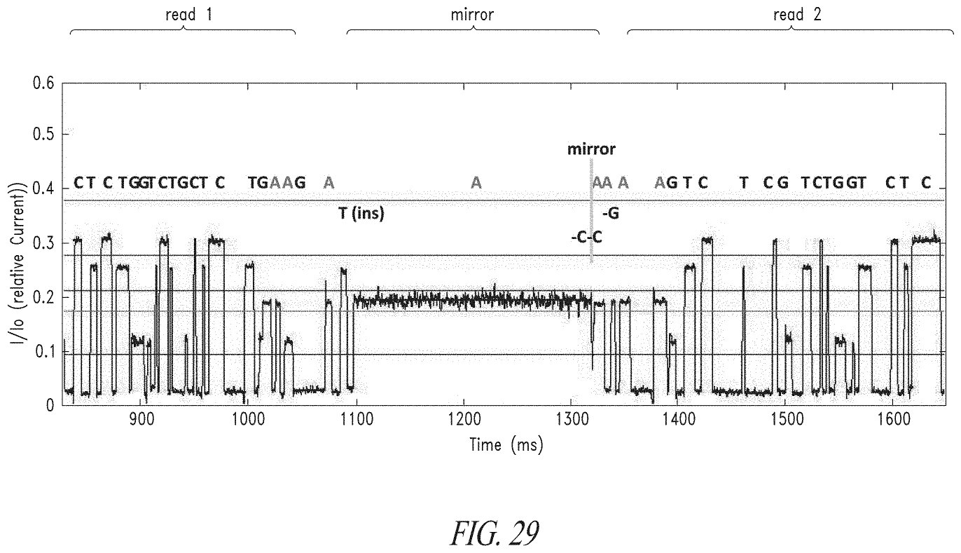

For sequencing by expansion, all steps of Xpandomer synthesis and processing were carried-out on the microfluidic chip. The Xpandomer extension conditions were as follows: 6% NMP, a 1:4 molar ratio of AZ,8-8 to AZ,43-43 PEM, 0.25M urea, 0.5M betaine, 80 μM XNTPs, 10 μg SSB, and C4760 polymerase for 30 minutes at 42° C. Following extension, the chip was washed. The Xpandomers were then cleaved by treating the chip with 200 μL 7.5M DCI for 30 minutes at 23° C. The chip was then neutralized and washed. The Xpandomers were then modified by adding 300 μL 125 mM succinate anhydride and incubating for 5 minutes at 23° C. Following wash, the Xpandomers were photo-cleaved from the chip (15 second UV treatment) and eluted with 100 μL of a solution containing 100 μM NaPO₄, 15% ACN, and 5% DMSO. Nanopore sequencing of the Xpandomer products was conducted as described in Example 2. A representative nanopore trace from this sample is presented in FIG. 29. The trace shows portions of two identical sequence reads, “read 1” and “read 2”, that reflect the sequence of the HIV2 library fragment (SEQ ID NO:16). The reads are separated by a signal that is produced by the cap oligo structure, referred to in the Figure as the “mirror”.

### Example 9

### Solid-State Xpandomer Synthesis with End-Capping on Acid-Resistant Magnetic Beads

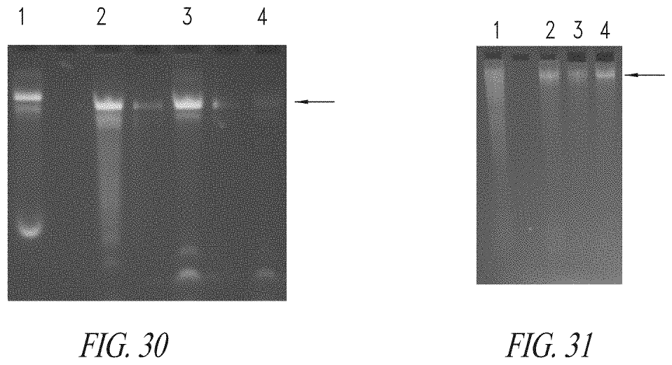

This example demonstrates that solid-state synthesis of Xpandomers on beads is at least as efficient as synthesis in-solution. Four different Xpandomer synthesis reactions were conducted: 1) in-solution synthesis (fluorescent SIMA dye on the extension oligonucleotide); 2) on-bead synthesis without end-capping (dye on the extension oligonucleotide); 3) on-bead synthesis with end-capping (dye on end cap terminal oligonucleotide); and 4) on-bead synthesis with a blocker oligonucleotide in place of the end cap. The extension oligonucleotide used in this experiment had the following sequence: 5′ [Azide]D₁₀[PC-Spacer]L₂₅Z₆[TCATAAGACGAACGGA (SEQ ID NO:4)] 3′, (in which “PC” represents a photocleavable spacer; “D” represents a PEG6 spacer; “L” represents a C2 spacer; and “Z” represents a C12 spacer). The beads were functionalized with an alkyne group and covalently bound to the extension oligonucleotide, as discussed herein and with reference to FIG. 5. 4 pmol on-bead extension oligonucleotide was hybridized to 4 pmol of 100mer template DNA+/− end cap oligonucleotide. The end cap included in reaction 3 had the following sequence: 3′ ddCTPRK[GCGTTAGGTCCCAGTTTTAC(SEQ ID NO:17)]W 5′ and the blocker oligonucleotide included in reaction 4 had the following sequence: 3′ RK[GCGTTAGGTCCCAGTGTTTTAC(SEQ ID NO:18)]X 5′, in which “R” represent amidite, “K” represents a G-clamp, “W” represent the SIMA dye, and “X” represents PEG3. A two-fold molar excess of cap or blocker oligo to template DNA was used. All extension reactions included: 50 mM Tris-HCl, 200 mM NH₄OAc, 20% PEG, 1M urea (0.25M for reaction 4), 5% NMS, 10 mM PEM, 0.26 μg/ul DPO4 polymerase variant, 1.6 mM MnCl₂, 100 μM dXTPs, and 300 μM polyphosphate. Reactions 3 and 4 also included 0.02% Tween and reaction 4 also included 0.5M betaine. Extension reactions were run for 60′ at 37° C. and extension products were analyzed by gel electrophoresis, as shown in FIG. 30. As can be seen, on-bead extension (lane 2) is just as efficient as in-solution extension (lane 1). Moreover, end-capping on-bead (lane 3, dye on end cap) is also extremely efficient.

### Example 10

### Solid-state Xpandomer Synthesis and Processing on Acid-Resistant Magnetic Beads

This example demonstrates efficient on-bead synthesis and processing of Xpandomers. Following primer extension reactions, Xpandomer products were processed by acid treatment to cleave the phosphoramidate bonds, generating expanded polymers. The expanded products were released from the beads by photocleavage and analyzed by gel electrophoresis.

Bead functionalization and extension oligonucleotide linkage was carried-out as described in Example 9. Template DNA was hybridized to the extension oligonucleotide at 1:1 molar ratio (4 pmol each). Extension reactions included: 50 mM Tris-HCl, 200 mM NH₄OAc, 50 mM TMACl, 50 mM GuCl, 20% PEG, 0.1M urea, 6% NMP, 15 mM PEM, 0.26 μg/ul DPO4 polymerase variant, 1.4 mM MnCl₂, 100 μM dXTPs, 0.05 μg/μl Kod single-stranded binding protein, 0.02% SDS, and 300 μM poly-phosphate. Extension reactions were run for 60′ at 37° C. Samples were then washed with buffer B (100 mM HEPES, 100 mM NaHPO₄, 5% Triton, and 15% DMF) treated with proteinase K for 5′ at 55° C. and washed again with buffer B. Samples were subjected to acid cleavage with 7.5M DCI/1% Triton, neutralized with buffer B, and modified with succinic anhydride in buffer B. Samples were then washed with buffer E (40% ACN) followed by photocleavage (1′ exposure to UV light) and released Xpandomer products were recovered and analyzed by gel electrophoresis, as shown in FIG. 31. Lane 1 represents Xpandomer products synthesized and processed in-solution, while lanes 2-4 represent Xpandomer products synthesized and processed on acid-resistant magnetic beads with different additives included in the elution buffer (100 mM PI in lane 2; 100 mM GuHCl in lane 3; and 100 mM HEPES in lane 4). As can be observed, the on-bead workflow shows improved results over the in-solution workflow, as the Xpandomer band is tighter, indicating that the samples are enriched for full-length product.

All of the U.S. patents, U.S. patent application publications, U.S. patent applications, foreign patents, foreign patent applications and non-patent publications referred to in this specification and/or listed in the Application Data Sheet, including but not limited to, U.S. Provisional Patent Application No. 62/808,768 filed on Feb. 21, 2019, and U.S. Provisional Patent Application No. 62/826,805 filed on Mar. 29, 2019, are incorporated herein by reference, in their entirety. Such documents may be incorporated by reference for the purpose of describing and disclosing, for example, materials and methodologies described in the publications, which might be used in connection with the presently described invention.

### SPECIFICALLY INCLUDED EMBODIMENTS

The following embodiments are specifically contemplated as part of the disclosure. This is not intended to be an exhaustive listing of potentially claimed embodiments included within the scope of the disclosure.

Embodiment 1. A method of synthesizing a copy of a nucleic acid template on a solid support comprising the steps of:

immobilizing a linker on the solid support, wherein the linker comprises a first end proximal to the solid support and a second end distal to the solid support, wherein the first end is coupled to a maleimide moiety and the second end is coupled to an alkyne moiety, and wherein the maleimide moiety is crosslinked to the solid support;

attaching an oligonucleotide primer to the linker, wherein the oligonucleotide primer comprises a nucleic acid sequence complementary to a portion of the 3′ end of the nucleic acid template, wherein the 5′ end of the oligonucleotide primer is coupled to an azide moiety, and wherein the azide moiety reacts with the alkyne moiety to form a triazole moiety;

providing a reaction mixture comprising the nucleic acid template, a nucleic acid polymerase, nucleotide substrates or analogs thereof, a suitable buffer, and, optionally, one or more additives, wherein the nucleic acid template specifically hybridizes to the oligonucleotide primer; and

performing a primer extension reaction to produce the copy of the nucleic acid template.

Embodiment 2. The method of Embodiment 1, wherein the maleimide moiety is crosslinked to the solid substrate by a photo-initiated proton abstraction reaction.

Embodiment 3. The method of Embodiment 1, wherein the solid substrate is comprised of polyolefin.

Embodiment 4. The method of Embodiment 3, wherein the polyolefin is a cyclic olefin copolymer (COC) or a polypropylene.

Embodiment 5. The method of Embodiment 1, wherein the nucleic acid template is a DNA template.

Embodiment 6. The method of Embodiment 5, wherein copy of the DNA template is an expandable polymer, wherein the expandable polymer comprises a strand of non-natural nucleotide analogs, and wherein the each of the non-natural nucleotide analogs is operably linked to the adjacent non-natural nucleotide analog by a phosphoramidate ester bond.

Embodiment 7. The method of Embodiment 6, wherein the expandable polymer is an Xpandomer.

Embodiment 8. The method of Embodiment 1, wherein the linker further comprises a spacer arm interposed between the first end and the second end, wherein the spacer arm comprises one or more monomers of ethylene glycol.

Embodiment 9. The method of Embodiment 1, wherein the linker further comprises a cleavable moiety.

Embodiment 10. The method of Embodiment 1, wherein the solid support is selected from the group consisting of a bead, a tube, a capillary, and a microfluidic chip.

Embodiment 11. A method of selectively modifying the 3′ end of a copy of a nucleic acid target sequence comprising the steps of:

providing a first oligonucleotide with a sequence complementary to a first sequence of the nucleic acid target sequence and a second oligonucleotide with a sequence complementary to a second sequence of the nucleic acid target sequence, wherein the first sequence of the nucleic acid target sequence is 3′ to the second sequence of the nucleic acid target sequence, wherein the first oligonucleotide provides an extension primer for a nucleic acid polymerase and the 5′ end of the second oligonucleotide is operably linked to a dideoxy nucleoside 5′ triphosphate, wherein the dideoxy nucleoside 5′ triphosphate provides a substrate for the nucleic acid polymerase;

providing a reaction mixture comprising the first and second oligonucleotides, the nucleic acid target sequence, the nucleic acid polymerase, nucleotide substrates or analogs thereof, a suitable buffer, and, optionally one or more additives, wherein the first and second oligonucleotides specifically hybridize to the nucleic acid target sequence; and

performing a primer extension reaction to produce the copy of the target sequence, wherein the 5′ end of the second oligonucleotide is operably linked to the 3′ end of the copy of the nucleic acid target sequence by the nucleic acid polymerase.

Embodiment 12. The method of Embodiment 11, wherein the dideoxy nucleoside 5′ triphosphate is operably linked to the 5′ end of the second oligonucleotide by a flexible linker.

Embodiment 13. The method of Embodiment 12, wherein the flexible linker comprises one or more hexyl (C₆) monomers.

Embodiment 14. The method of Embodiment 13, wherein the second oligonucleotide comprises one or more 2′methoxyribonucleic acid analogs.

Embodiment 15. The method of Embodiment 11, wherein the 3′ end of the second oligonucleotide is immobilized on a first solid support.

Embodiment 16. The method of Embodiment 15, further comprising the step of washing the first solid support to purify the copy of the nucleic acid target operably linked to the second oligonucleotide.

Embodiment 17. The method of Embodiment 11, wherein the first oligonucleotide is immobilized to a first solid support.

Embodiment 18. The method of Embodiment 17, further comprising the steps of releasing the copy of the nucleic acid target sequence from the first solid support and contacting the copy of the nucleic acid target sequence with a third oligonucleotide, wherein the third oligonucleotide has a sequence that is complementary to the sequence of the second oligonucleotide, wherein the third oligonucleotide specifically hybridizes with the second oligonucleotide, and wherein the 5′ end of the third oligonucleotide is immobilized on a second solid support.

Embodiment 19. The method of Embodiment 18, further comprising the step of washing the second solid support to purify the copy of the nucleic acid target sequence operably linked at the 3′ end to the second oligonucleotide.

Embodiment 20. The method of Embodiment 11, wherein the second oligonucleotide comprises one or more nucleotide analogs that increase the binding affinity of the second oligonucleotide for the nucleic acid target sequence.

Embodiment 21. The method of Embodiment 11, wherein the second oligonucleotide is complementary to a heterologous nucleic acid sequence operably linked to the 5′ end of the nucleic target sequence.

Embodiment 22. The method of Embodiment 11, wherein the nucleic acid target sequence is single-stranded DNA and the copy of the target sequence is an expandable polymer, wherein the expandable polymer comprises a strand of non-natural nucleotide analogs, and wherein the each of the non-natural nucleotide analogs is operably linked to the adjacent non-natural nucleotide analog by a phosphoramidate ester bond.

Embodiment 23. The method of Embodiment 11 or Embodiment 18, wherein the first and second solid supports are selected from the group consisting of a bead, a tube, a capillary, and a microfluidic chip.

Embodiment 24. A method for producing a library of single-stranded DNA template constructs, wherein the each of the template constructs comprises two copies of the same strand of a DNA target sequence, comprising the steps of:

providing a population of DNA Y adaptors, wherein each of the Y adaptors comprises a first oligonucleotide and a second oligonucleotide, wherein the 3′ region of the first oligonucleotide and the 5′ region of the second oligonucleotide form a double-stranded region by sequence complementarity, wherein the 5′ region of the first oligonucleotide and the 3′ region of the second oligonucleotide are single-stranded and comprise binding sites for oligonucleotide primers, and wherein the ends of the single-stranded regions of the first and second oligonucleotides are optionally immobilized on a solid substrate;

providing a population of double-stranded DNA molecules, wherein each of the double-stranded DNA molecules comprises a first strand and a second strand, wherein a first end of each of the double-stranded DNA molecules is compatible with the double-stranded end of the Y adaptors;

providing a population of cap primer adaptors, wherein each of the cap primer adaptors is comprised of a first, a second, and a third oligonucleotide, wherein the second oligonucleotide is interposed between the first and the third oligonucleotide, wherein the first, second, and third oligonucleotides are operably linked at the 5′ ends of the first and the third oligonucleotides and the 3′ end of the second oligonucleotides by a chemical brancher, wherein a portion of the sequence of the first oligonucleotide is identical to a portion of the sequence of the third oligonucleotide, wherein a portion of the sequence of the second oligonucleotide is the reverse complement of the portions of the sequences of the first and third oligonucleotides, and wherein the 5′ end of the second oligonucleotide and the 3′ end of the third oligonucleotide form a double-stranded region that is compatible with a second end of each of the double-stranded DNA molecules;

ligating the second end of each of the double-stranded DNA molecules to the 5′ end of the second oligonucleotide and the 3′ end of the third oligonucleotide of one of the cap primer adaptors;

ligating the first end of each of the double-stranded DNA molecules to the double-stranded end of one of the DNA Y adaptors;

extending from the 3′ end of the first oligonucleotide of each of the ligated cap primer adaptors with a DNA polymerase, wherein the first strand of the ligated double-stranded DNA molecule provides a template for the DNA polymerase, and wherein the DNA polymerase produces a third strand that comprises the reverse complement of the sequences of the first strand of the double-stranded DNA molecule and the sequence of the first oligonucleotide of the Y adaptor; and

digesting from the 5′ end of each of the first oligonucleotides of the ligated Y adaptors with an exonuclease, wherein the digesting removes the first oligonucleotide, the first strand of the double-stranded DNA molecule, and the second oligonucleotide of the cap primer adaptor to produce a single-stranded template construct, wherein each of the single-stranded template constructs comprises two template molecules each comprising the sequence of the second strand of the double-stranded DNA molecule, and wherein the two template molecules are operably linked by the first and third oligonucleotides of the cap primer adaptor.

Embodiment 25. A library of single-stranded DNA template constructs, wherein each of the template constructs comprises a first and a second copy of the same strand of a DNA target sequence, wherein the first and the second copies of the target sequence are operably linked; and wherein the library of single-stranded DNA template constructs is produced by the method of Embodiment 24.

Embodiment 26. A method of producing a library of mirrored Xpandomer molecules, wherein each of the Xpandomer molecules comprises two copies of the same strand of a DNA target sequence, comprising the steps of:

providing the library of single-stranded DNA template constructs of Embodiment 25;

providing a population of first extension oligonucleotides complementary to the single-stranded portion of the first strand of the Y adaptor and a population of second extension oligonucleotides complementary to the single-stranded portion of the second strand of the Y adaptor, and wherein the first or second extension oligonucleotides are optionally immobilized on a solid substrate;

specifically hybridizing the library of single-stranded DNA template constructs to the population of first and second extension oligonucleotides;

providing a population of cap brancher constructs, wherein the cap brancher constructs comprise a first oligonucleotide operably linked to a second oligonucleotide, wherein the first and second oligonucleotides comprise sequences complementary to a portion of the sequences of the first and third oligonucleotides of the cap primer adaptor constructs, and wherein the first and second oligonucleotides of the cap brancher constructs provide free 5′ nucleoside triphosphate moieties;

specifically hybridizing the population of cap brancher constructs to the population of single-stranded DNA template constructs; and

performing primer extension reactions to produce Xpandomer copies of the first and second copies of the DNA target sequences, wherein the Xpandomer copies are operably linked by the cap brancher constructs.

Embodiment 27. A method for producing a library of tagged double-stranded DNA amplicons on a solid support, comprising the steps of:

providing a population of double-stranded DNA molecules, wherein each of the double-stranded DNA molecules comprises a first strand specifically hybridized to a second strand;

providing forward PCR primers and reverse PCR primers, wherein the forward PCR primers comprise a first 5′ heterologous tag sequence operably linked to a 3′ sequence complementary to a portion of the 3′ end of the second stand of the double-stranded DNA molecules, and wherein the reverse PCR primers comprise a second 5′ heterologous tag sequence operably linked to a 3′ sequence complementary to a portion of the 3′ end of the first strand of the double-stranded DNA molecules;

performing a first PCR reaction, wherein the population of double-stranded DNA molecules is amplified to produce a population of first DNA amplicon products, wherein the first DNA amplicon products comprise the first heterologous sequence tag on a first end and the second heterologous sequence tag on a second end;

providing a capture oligonucleotide structure immobilized on a solid support, wherein the capture oligonucleotide structure comprises a first end and a second end, wherein the first end is covalently attached to the solid support, wherein the second end comprises a capture oligonucleotide comprising a sequence complementary to a portion of the second heterologous sequence tag of the first population of DNA amplicon products, and wherein the capture oligonucleotide structure further comprises a cleavable element interposed between the first end and the capture oligonucleotide; and

performing a second PCR reaction comprising the population of first DNA amplicon products, forward primers comprising a sequence complementary to the sequence of one of the strands of the first heterologous sequence tag, and reverse primers comprising a sequence complementary to one of the strands of the second heterologous sequence tag, wherein a first strand of the population of first DNA amplicon products specifically hybridizes to the capture oligonucleotide, and wherein the second PCR reaction produces a population of immobilized DNA amplicon products, wherein a second strand of the immobilized DNA amplicon products is operably linked to the solid support.

Embodiment 28. A method for producing a library of single-stranded DNA template constructs, wherein the each of the template constructs comprises two copies of the same strand of a DNA target sequence, comprising the steps of:

providing the library of DNA amplicon products immobilized on a solid support of Embodiment 27;

providing a population of cap primer adaptors, wherein each of the cap primer adaptors is comprised of a first, a second, and a third oligonucleotide, wherein the second oligonucleotide is interposed between the first and the third oligonucleotide, wherein the first, second, and third oligonucleotides are operably linked at the 5′ ends of the first and the third oligonucleotides and the 3′ end of the second oligonucleotides by a chemical brancher, wherein a portion of the sequence of the first oligonucleotide is identical to a portion of the sequence of the third oligonucleotide, wherein a portion of the sequence of the second oligonucleotide is the reverse complement of the portions of the sequences of the first and third oligonucleotides, and wherein the 5′ end of the second oligonucleotide and the 3′ end of the third oligonucleotide form a double-stranded region that is compatible with a free end of each of the tagged immobilized DNA amplicon products;

ligating the free end of each of the immobilized DNA amplicon products to the 5′ end of the second oligonucleotide and the 3′ end of the third oligonucleotide of the cap primer adaptors;

extending from the 3′ end of each of the first oligonucleotide of the cap primer adaptors with a DNA polymerase, wherein the second strand of the immobilized DNA amplicon products provide a template for the DNA polymerase, and wherein the DNA polymerase produces a third strand, wherein the third strand is a copy of the second strand;

cleaving the cleavable element of each of the capture oligonucleotide structures, wherein the cleaving releases the DNA amplicon products from the solid support and produces a free 5′ end on the second strand of each of the DNA amplicon products; and

digesting from the free 5′ end of the cleaved second strand of each of the DNA amplicon products with an exonuclease, wherein the digesting removes the second strand of the DNA amplicon product and the second oligonucleotide of the cap primer adaptor to produce a library of single-stranded template constructs, wherein each of the single-stranded template constructs comprises two copies of the first strand of the DNA amplicon products operably linked by the first and third oligonucleotides of the cap primer adaptor.

Embodiment 29. A library of single-stranded DNA template constructs, wherein the each of the template constructs comprises a first and a second copy of the same strand of a DNA target sequence, wherein the first and second copies of the DNA target sequence are operably linked, and wherein the library of single-stranded DNA template constructs is produced by the method of Embodiment 28.

Embodiment 30. A method of producing a library of mirrored Xpandomer molecules, wherein each of the Xpandomer molecules comprises two copies of the same strand of a DNA target sequence, comprising the steps of:

providing the library of single-stranded DNA template constructs of Embodiment 29;

providing a population of extension oligonucleotides complementary to the second tag of the DNA amplicon products, wherein the extension oligonucleotides are immobilized on a solid substrate;

specifically hybridizing the single-stranded DNA template constructs to the extension oligonucleotides;

providing a population of cap brancher constructs, wherein the cap brancher constructs comprise a first oligonucleotide operably linked to a second oligonucleotide, wherein the first and second oligonucleotides comprise sequences complementary to a portion of the sequences of the first and third oligonucleotides of the cap primer adaptor constructs and wherein the first and second oligonucleotides of the cap brancher constructs provide free 5′ nucleoside triphosphate moieties;

specifically hybridizing the population of cap brancher constructs with the population of DNA template constructs; and

performing primer extension reactions to produce Xpandomer copies of the first and second copies of the DNA target sequences, wherein the Xpandomer copies are operably linked to the cap brancher constructs.

Embodiment 31. The method of Embodiment 30, wherein the capture oligonucleotide structure and the extension oligonucleotides are immobilized on the same solid support, wherein the extension oligonucleotides comprise a cleavable hairpin structure, and wherein the cleavable hairpin structure is cleaved during the cleaving step to provide binding sites for the DNA amplicon products.

Embodiment 32. The method of Embodiment 30, wherein the capture oligonucleotide structure is immobilized on a first substrate of a first chamber of a microfluidic card and the extension oligonucleotides are immobilized on a second substrate of a second chamber of the microfluidic card and wherein the first chamber is configured to produce the population of single-stranded DNA template constructs and the second chamber is configured to produce the population of Xpandomer copies of the single-stranded DNA template constructs.

Embodiment 33. The method of Embodiment 30, wherein the capture oligonucleotide structure is immobilized on a bead support and the extension oligonucleotides are immobilized on a COC chip support, wherein the bead support is configured to produce the population of single-stranded DNA template constructs and the COC chip support is configured to produce the population of Xpandomer copies of the DNA template constructs.

Embodiment 34. The method of Embodiment 30, wherein the capture oligonucleotide structure and the extension oligonucleotides are immobilized on a bead support, wherein the bead support is configured to produce the population of single-stranded DNA template constructs and the population of Xpandomer copies of the DNA template constructs.

Embodiment 35. The method of Embodiment 30, wherein the extension oligonucleotides are provided by a branched oligonucleotide structure, wherein the branched oligonucleotide structure comprises a first extension oligonucleotide operably linked to a second extension oligonucleotide by a chemical brancher, wherein the first extension oligonucleotide comprises a leader sequence, a concentrator sequence and a first cleavable moiety interposed between the chemical brancher and the leader and the concentrator sequences and wherein the second extension oligonucleotide comprises a second cleavable moiety.

What is claimed is:

1. A method of synthesizing a copy of a nucleic acid template on a solid support comprising the steps of:
(a) immobilizing a linker on the solid support, wherein the linker comprises a first end proximal to the solid support and a second end distal to the solid support, wherein the first end is coupled to a maleimide moiety and the second end is coupled to an alkyne moiety, and wherein the maleimide moiety is crosslinked to the solid support;
(b) attaching an oligonucleotide primer to the linker, wherein the oligonucleotide primer comprises a nucleic acid sequence complementary to a portion of the 3′ end of the nucleic acid template, wherein the 5′ end of the oligonucleotide primer is coupled to an azide moiety, and wherein the azide moiety reacts with the alkyne moiety to form a triazole moiety;
(c) providing a reaction mixture comprising the nucleic acid template, a nucleic acid polymerase, nucleotide substrates or analogs thereof, a suitable buffer, and, optionally, one or more additives, wherein the nucleic acid template specifically hybridizes to the oligonucleotide primer; and
(d) performing a primer extension reaction to produce the copy of the nucleic acid template.

2. The method of claim 1, wherein the maleimide moiety is crosslinked to the solid substrate by a photo-initiated proton abstraction reaction.

3. The method of claim 1, wherein the solid substrate is comprised of polyolefin.

4. The method of claim 3, wherein the polyolefin is a cyclic olefin copolymer (COC) or a polypropylene.

5. The method of claim 1, wherein the nucleic acid template is a DNA template.

6. The method of claim 5, wherein copy of the DNA template is an expandable polymer, wherein the expandable polymer comprises a strand of non-natural nucleotide analogs, and wherein the each of the non-natural nucleotide analogs is operably linked to the adjacent non-natural nucleotide analog by a phosphoramidate ester bond.

7. The method of claim 6, wherein the expandable polymer is an Xpandomer.

8. The method of claim 1, wherein the linker further comprises a spacer arm interposed between the first end and the second end, wherein the spacer arm comprises one or more monomers of ethylene glycol.

9. The method of claim 1, wherein the linker further comprises a cleavable moiety.

10. The method of claim 1, wherein the solid support is selected from the group consisting of a bead, a tube, a capillary, and a microfluidic chip.

11. A method of selectively modifying the 3′ end of a copy of a nucleic acid target sequence comprising the steps of:
(a) providing a first oligonucleotide with a sequence complementary to a first sequence of the nucleic acid target sequence and a second oligonucleotide with a sequence complementary to a second sequence of the nucleic acid target sequence, wherein the first sequence of the nucleic acid target sequence is 3′ to the second sequence of the nucleic acid target sequence, wherein the first oligonucleotide provides an extension primer for a nucleic acid polymerase and the 5′ end of the second oligonucleotide is operably linked to a dideoxy nucleoside 5′ triphosphate, wherein the dideoxy nucleoside 5′ triphosphate provides a substrate for the nucleic acid polymerase;
(b) providing a reaction mixture comprising the first and second oligonucleotides, the nucleic acid target sequence, the nucleic acid polymerase, nucleotide substrates or analogs thereof, a suitable buffer, and, optionally one or more additives, wherein the first and second oligonucleotides specifically hybridize to the nucleic acid target sequence; and
(c) performing a primer extension reaction to produce the copy of the target sequence, wherein the 5′ end of the second oligonucleotide is operably linked to the 3′ end of the copy of the nucleic acid target sequence by the nucleic acid polymerase.

12. The method of claim 11, wherein the dideoxy nucleoside 5′ triphosphate is operably linked to the 5′ end of the second oligonucleotide by a flexible linker.

13. The method of claim 12, wherein the flexible linker comprises one or more hexyl (C₆) monomers.

14. The method of claim 13, wherein the second oligonucleotide comprises one or more 2′methoxyribonucleic acid analogs.

15. The method of claim 11, wherein the 3′ end of the second oligonucleotide is immobilized on a first solid support.

16. The method of claim 15, further comprising the step of washing the first solid support to purify the copy of the nucleic acid target operably linked to the second oligonucleotide.

17. The method of claim 11, wherein the first oligonucleotide is immobilized to a first solid support.

18. The method of claim 17, further comprising the steps of releasing the copy of the nucleic acid target sequence from the first solid support and contacting the copy of the nucleic acid target sequence with a third oligonucleotide, wherein the third oligonucleotide has a sequence that is complementary to the sequence of the second oligonucleotide, wherein the third oligonucleotide specifically hybridizes with the second oligonucleotide, and wherein the 5′ end of the third oligonucleotide is immobilized on a second solid support.

19. The method of claim 18, further comprising the step of washing the second solid support to purify the copy of the nucleic acid target sequence operably linked at the 3′ end to the second oligonucleotide.

20. The method of claim 11, wherein the second oligonucleotide comprises one or more nucleotide analogs that increase the binding affinity of the second oligonucleotide for the nucleic acid target sequence.

21. The method of claim 11, wherein the second oligonucleotide is complementary to a heterologous nucleic acid sequence operably linked to the 5′ end of the nucleic target sequence.

22. The method of claim 11, wherein the nucleic acid target sequence is single-stranded DNA and the copy of the target sequence is an expandable polymer, wherein the expandable polymer comprises a strand of non-natural nucleotide analogs, and wherein the each of the non-natural nucleotide analogs is operably linked to the adjacent non-natural nucleotide analog by a phosphoramidate ester bond.

23. The method of claim 18, wherein the first and second solid supports are selected from the group consisting of a bead, a tube, a capillary, and a microfluidic chip.


SEQUENCE LISTING (18 sequences)

SEQ ID NO: 1 - DNA, 14 bp, Artificial Sequence
  Single-stranded template
tccggaagctagcc

SEQ ID NO: 2 - DNA, 23 bp, Artificial Sequence
  Terminal oligonucleotide
ttgtaggaaggccagatcttccc

SEQ ID NO: 3 - DNA, 23 bp, Artificial Sequence
  Oligonucleotide
ctgcgttaggtcacagtgtttac

SEQ ID NO: 4 - DNA, 16 bp, Artificial Sequence
  Oligonucleotide
tcataagacgaacgga

SEQ ID NO: 5 - DNA, 116 bp, Artificial Sequence
  Library Fragment - plus strand
gggaagatctggccttcctacaagggaaggccagggaattttcttcagagcagaccagagccaacagccccaccagaagagagcttcaggtctggggtagtccgttcgtcttatga

SEQ ID NO: 6 - DNA, 116 bp, Artificial Sequence
  Library Fragment - minus strand
tcataagacgaacggactaccccagacctgaagctctcttctggtggggctgttggctctggtctgctctgaagaaaattccctggccttcccttgtaggaaggccagatcttccc

SEQ ID NO: 7 - DNA, 38 bp, Artificial Sequence
  Primer
tcataagacgaacggagactctaccccagacctgaagc

SEQ ID NO: 8 - DNA, 42 bp, Artificial Sequence
  Primer
cgtcgtagctccatctgtcaaagggaagatctggccttccta

SEQ ID NO: 9 - DNA, 142 bp, Artificial Sequence
  Tagged Fragment - plus strand
cgtcgtagctccatctgtcaaagggaagatctggccttcctacaagggaaggccagggaattttcttcagagcagaccagagccaacagccccaccagaagagagcttcaggtctggggtagagtctccgttcgtcttatga

SEQ ID NO: 10 - DNA, 142 bp, Artificial Sequence
  Tagged Fragment - minus strand
tcataagacgaacggagactctaccccagacctgaagctctcttctggtggggctgttggctctggtctgctctgaagaaaattccctggccttcccttgtaggaaggccagatcttccctttgacagatggagctacgacg

SEQ ID NO: 11 - DNA, 20 bp, Artificial Sequence
  Tag
tcataagacgaacggagact

SEQ ID NO: 12 - DNA, 28 bp, Artificial Sequence
  Bystander oligonucleotide
  misc_feature (19)..(20): N = Uracil
tcataagacgaacggaganntccgttcg

SEQ ID NO: 13 - DNA, 20 bp, Artificial Sequence
  Oligonucleotide
tcataagacgaacggagact

SEQ ID NO: 14 - DNA, 18 bp, Artificial Sequence
  Oligonucleotide primer
tcataagacgaacggaga

SEQ ID NO: 15 - DNA, 21 bp, Artificial Sequence
  End cap
gcgttaggtcccagtgtttac

SEQ ID NO: 16 - DNA, 21 bp, Artificial Sequence
  HIV2 library fragment
ctctggtctgctctgaagaac

SEQ ID NO: 17 - DNA, 20 bp, Artificial Sequence
  End cap
cattttgaccctggattgcg

SEQ ID NO: 18 - DNA, 22 bp, Artificial Sequence
  Blocker oligonucleotide
cattttgtgaccctggattgcg